(19) United States
(12) Patent Application Publication
(10) Pub. No.: US 2023/0220001 A1
(43) Pub. Date: Jul. 13, 2023

(54) METHOD FOR SYNTHESIS OF THIOETHER-CONTAINING PEPTIDES

(71) Applicant: HEIDELBERG PHARMA RESEARCH GMBH, Ladenburg (DE)

(72) Inventors: Hendrik GRUSS, Ladenburg (DE); Christian LUTZ, Ladenburg (DE); Roderich SÜSSMUTH, Berlin (DE); Guiyang YAO, Berlin (DE); Caroline KNITTEL, Berlin (DE); Simone KOSOL, Berlin (DE); Andi MAINZ, Berlin (DE)

(73) Assignee: HEIDELBERG PHARMA RESEARCH GMBH, Ladenburg (DE)

(21) Appl. No.: 18/001,173

(22) PCT Filed: Jun. 9, 2021
(86) PCT No.: PCT/EP2021/065392

§ 371 Date: Dec. 8, 2022

(30) Foreign Application Priority Data
Jun. 9, 2020 (EP) 20178980.7

(51) Int. Cl. C07K 1/113; C07D 209/20; C07K 7/64; A61K 47/68

(52) U.S. Cl. CPC C07K 1/113; C07D 209/20; C07K 7/64; A61K 47/6803

(57) ABSTRACT

The present invention relates to a method of formation of a sulphur bridge between tryptophan and cysteine in solid phase peptide synthesis under iodine treatment. The invention also relates to the resulting compounds of the method and their respective use.

The present invention relates to a method of formation of a sulphur bridge between tryptophan and cysteine in solid phase peptide synthesis under iodine treatment. The invention also relates to the resulting compounds of the method.

### BACKGROUND OF THE INVENTION

Alpha-amanitin, a slow acting toxin (LD₅₀=50-100 μg/kg) (T. Wieland, Peptides of Poisonous Amanita Mushrooms (Eds.: A. Rich), Wiley-VCH, Weinheim, 1986), is a selective inhibitor of RNA polymerase II (Pol II). Its bicyclic octapeptide structure contains a 6-hydroxy-tryptathionine-(R)-sulfoxide cross-link (FIG. 4A, FIG. 9). Studies on structure activity relationship have shown that the tryptathionine crosslink is essential for this bioactivity while the 6-hydroxy group of Trp and the sulfoxide are not essential for cytotoxicity. Thus, based on the previous research, antibody-toxin conjugates (FIG. 4B, FIG. 9) have been developed for cancer therapy (Pahl et al. Drug Discov Today: Technol, 2018, 30, 85-89). In this approach the amanitin component serves as a toxic payload of the conjugate which is delivered to the target cell and there released. Hence, cyclic peptides with a similar architecture compared to amanitin are of interest to serve as the toxin-component of antibody-toxin-conjugates (FIG. 4).

The synthesis of the amino acid tryptathionine has been investigated for many years and there are three basic synthetic approaches for their formation. The early syntheses of tryptathionine consist of reacting indoles with sulfenyl chlorides (Anderson, A. A. Shelat, R. K. Guy, J Org Chem 2005, 70, 4578-4584). Although this approach has been used quite extensively, laborious protection and deprotection strategies involved disfavoured it compared to the Savige-Fontana reaction (Savige et al. J. Chem. Soc., Chem. Commun. 1976, 600-601), which is an additional synthetic method to access the tryptathionine moiety. Using the Savige-Fontana reaction, initial structure-activity relationships of various derivatives of amanitin were performed as well as the first total synthesis of alpha-amanitin (Zanotti et al. Chem-Eur J 2001, 7, 1479-1485; Matinkhoo et al. J Am Chem Soc 2018, 140, 6513-651).

Previously, the iodine-mediated thionation of a non-protected Trp sidechain and trityl-protected Cys has been described at the example of some model peptides (Sieber et al. Helv Chim Acta 1980, 63, 2358-2363). This approach has been applied to the synthesis of phalloidin and derivatives (Schuresko et al. Angew Chem Int Edit 2007, 46, 3547-3549; Yao et al. Chem-Eur J 2019, 25, 8030-8034) but was not described for amanitin-type peptides and sequences derived thereof, since different length, order, and intramolecular hydrogen bonds of the peptide sequences formed different results and yields.

Based on the above-mentioned state of the art, the objective of the present invention is to provide means and methods to form a tryptathione-type sulphur bridge in solid phase peptide synthesis, for the synthesis of cyclic peptides, particularly for the synthesis of amatoxin and derivatives. This objective is attained by the subject-matter of the independent claims of the present specification.

### SUMMARY OF THE INVENTION

The invention relates to a method for the preparation of a compound of formula (I)

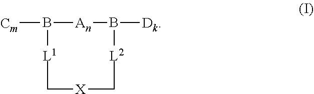

### DESCRIPTION OF THE FIGURES

FIG. 1 Solid phase peptide synthesis of mono cyclic pentapeptide.

FIG. 2 Solid phase peptide synthesis of mono cyclic peptide.

FIG. 3 Solid phase peptide synthesis of mono cyclic peptide.

FIG. 4 Structure of (A) alpha-amanitin and with A ring and B ring (B) schematic example of an antibody-toxin-conjugate.

FIG. 5 Solution phase peptide synthesis of mono cyclic pentapeptide.

FIG. 6 Synthesis of Fmoc-L-Trp(6-OBn)-OH. a) POCl₃, DMF; b) (Boc)₂O, DMAP, DCM; c) Boc-2-phosphonoglycine-methyl dimethyl ester, DBU (1,8-Diazabicyclo[5.4.0]undec-7-ene), DCM, 0° C. to rt, 16 h; d) H₂ (20 bar), 4 mol % (R)-MonoPhos, 2 mol % [Rh(COD)₂BF₄], DCM 3 h, rt, ee=99%; e) 0.5N LiOH/MeOH/THF (1:1:1), 2 h; f) TFA/DCM (1:1), 2 h; g) Fmoc-OSu, NaHCO₃, 1,4-dioxane/H₂O, 12 h.

FIG. 7 Synthesis of Cbz-L-Trp(Boc)(6OBn)-OMe. A) Cbz-2-phosphonoglycine-methyl dimethyl ester, DBU, DCM, 0° C. to rt, 16 h; b) H₂ (20 bar), 4 mol % (R)-MonoPhos, 2 mol % [Rh(COD)₂BF₄], DCM 4 h, rt, ee=99%.

FIG. 8 Synthesis of Boc-D-Trp(Boc)(6-OBn)-OMe, H₂ (20 bar), 4 mol % (S)-MonoPhos, 2 mol % [Rh(COD)₂BF₄], DCM 3 h, rt, ee=99%.

FIG. 9 shows the structural formulae of different amatoxins. The numbers in bold type (1 to 8) designate the standard numbering of the eight amino acids forming the amatoxin. The standard designations of the atoms in amino acids 1, 3 and 4 are also shown (Greek letters a to y, Greek letters a to 6, and numbers from 1′ to 7′, respectively). A ring and B ring are labelled.

### DETAILED DESCRIPTION OF THE INVENTION

### Terms and Definitions

Amino acid sequences are given from amino to carboxyl terminus. Capital letters for sequence positions refer to L-amino acids in the one-letter code (Stryer, Biochemistry, 3^{rd} ed. p. 21).

Lower case letters for amino acid sequence positions refer to the corresponding D- or (2R)-amino acids.

The term “protecting group” in the context of the present specification relates to a moiety covalently attached to a functional group (particularly the carboxylic acid moiety, the amino moiety or the hydroxyl moiety of the molecules discussed herein) that can be selectively attached to the functional group and selectively removed without affecting the integrity or chiral orientation of the carbon backbone of the molecule the protecting group is attached to, nor cleaving particular other protecting groups attached to the molecule.

The term “deprotection agent” in the context of the present specification relates to an agent which is able to cleave a certain protecting group. The skilled person is able to select the deprotection agent according to the protecting group. The conditions under which the protecting group is cleavable constitute the deprotection agent, e.g. if the protecting group is cleavable under acidic conditions, then the deprotection agent is an acid.

A comprehensive review of modern protecting group chemistry, particularly as it pertains to the compounds disclosed herein, is available in Peter G. M. Wuts, Greene's Protective Groups in Organic Synthesis, 5th Edition, Wiley 2014.

U.S. Pat. No. 6,693,178 B2—“Protecting groups useful in the synthesis of polysaccharides, natural products, and combinatorial libraries” and US 20160024143 A1—“Deprotection method” are incorporated herein by reference.

Standard convention of organic chemistry, by which a non-designated position in a formula is deemed to be a saturated carbon, is followed herein.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). If not specified, the amino acid is an L-amino acid.

The term unsubstituted Cₙ alkyl when used herein in the narrowest sense relates to the moiety —CₙH₂ₙ— if used as a bridge between moieties of the molecule, or —CₙH₂ₙ₊₁ if used in the context of a terminal moiety.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂(iso-propyl, i-pr), but is butyl —C₄H₉, —(CH₂)₃CH₃, —CHCH₃CH₂CH₃, —CH₂CH(CH₃)₂ or —C(CH₃)₃.

The term heteroaryl in the context of the present specification relates to a cyclic aromatic C₅-C₁₀ hydrocarbon that comprises at least one heteroatom (e.g. N, O, S), particularly one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. A heteroaryl in the context of the specification additionally may be substituted by one or more alkyl groups.

The term substituted heteroaryl in its broadest sense refers to a heteroaryl as defined above in the broadest sense, which is covalently linked to an atom that is not carbon or hydrogen, particularly to an atom selected from N, O, F, B, Si, P, S, Cl, Br and I, which itself may be—if applicable-linked to one or several other atoms of this group, or to hydrogen, or to an unsaturated or saturated hydrocarbon (alkyl or aryl in their broadest sense). In a narrower sense, substituted heteroaryl refers to a heteroaryl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, chloride Cl, bromide Br, iodide I, phosphonate PO₃H₂, PO₃R₂, phosphate OPO₃H₂ and OPO₃R₂, sulfhydryl SH, sulfalkyl SR, sulfoxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The method of the invention relates to solid-phase peptide synthesis, wherein a sulphur bridge is formed under oxidizing conditions, particularly under iodine treatment.

A first aspect of the invention relates to a method for preparation of a compound of formula (I)

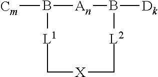

wherein
a compound of formula (II)

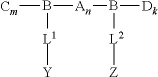

is reacted with iodine (I₂), iodine monochloride (ICI), iodine monobromide (IBr), N-bromosuccinimide, N-iodosuccinimide or Bis(pyridine)iodonium tetrafluoroborate, particularly with iodine, in a reaction step (a) yielding a compound of formula (I); wherein
   Z is selected from SH, S-trityl (STrt), S-acetamidomethyl (SAcm), S-diphenylmethyl (SDpm), S-monomethoxytrityl (SMmt), and S-tert-butyl (S^{t}Bu), particularly Z is selected from SH, S-trityl (STrt), S-acetamidomethyl (SAcm), S-diphenylmethyl (SDpm), S-monomethoxytrityl (SMmt), and S-tert-butyl (S^{t}Bu), most particularly Z is STrt;
   Y is an unsubstituted or hydroxyl-, halogen-, halogenated carbon-, alkynyl-, olefin-, cyano-, protected carboxylate and/or carboxyamide-substituted indole, particularly is an unsubstituted or hydroxyl-, halogen-, halogenated carbon-, alkynyl-, olefin-, cyano-, protected carboxylate and/or carboxyamide-substituted indole;
   L¹ is an unsubstituted or Me-substituted C₁-C₂ alkyl linker, particularly L¹ is CH₂;
   L² is an unsubstituted or Me-substituted C₁-C₂ alkyl linker, particularly L¹ is CH₂ or —C(CH₃)₂—;
   B is an α-amino acid, a β-amino acid backbone, a α-methyl amino acid, particularly B is an α-amino acid backbone (NH—CHR—C═O with R being L¹ or L²)
   every A is independently selected from a proteinogenic and a non-proteinogenic α-amino acid in L- or D-conformation or a β-amino acid, particularly every A is independently selected from a proteinogenic and a non-proteinogenic α-amino acid in L- or D-conformation;
   n is an integer selected from 2, 3, and 4;
   with the proviso that the ring

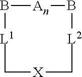

consists of 14 to 26 bonds, particularly of 15 to 21 bonds, more particularly of 18 bonds;
every C and D are independently selected from a proteinogenic and a non-proteinogenic α-amino acid in L- or D-conformation, wherein either C or D of formula (II) is connected to a resin or has a protected N-terminus;
m and k are independently selected from an integer between 0 and 4, particularly wherein the sum of m and k is an integer between 0 and 4;
X is either (-(-indole-S—) or (—S-indole-)-) with the indole being an unsubstituted or hydroxyl-, halogen-, halogenated carbon-, alkynyl-, olefin-, cyano-, protected carboxylate and/or carboxyamide-substituted indole, wherein optionally the sulphur atom of X can be subsequently oxidized.

Said compound may be represented by either formula (Ia) or formula (Ib)

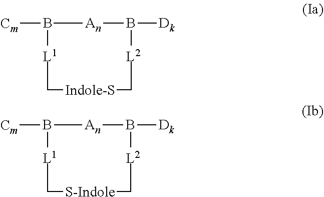

particularly with the proviso that the direction from left to right is either from N-terminus to C-terminus or from C-terminus to N-terminus.

If not stated differently, the reaction with iodine is performed as described in the materials and methods section under “₂-mediated cyclization”. As iodine source reagents, iodine monochloride (CAS: 7790-99), iodine monobromide (CAS: 7789-33-5), Bis(pyridine)iodonium tetrafluoroborate (CAS: 15656-28-7), N-iodosuccinimide (NIS) and N-bromosuccinimide (NBS) have been used successfully.

The solid-phase peptide synthesis is performed on a solid support. The solid support consists of small, polymeric resin beads functionalized with reactive groups. The sulphur bridge formation of the invention may be performed when the peptide is still linked to the resin or after cleavage from the resin. If performed after cleavage, the N-terminus of the peptide is protected with an amino-protecting group.

In certain embodiments, resin loading is around 0.3 mmol/g. With higher resin loading, side reactions would be promoted.

An α-L-amino acid backbone is of formula

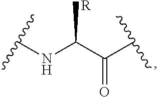

wherein R is the amino acid side chain.

In certain embodiments, either C or D of formula (II) is connected to a resin and is reacted with iodine (I₂). In certain embodiments, either C or D of formula (II) is connected to a resin and is reacted with iodine at a concentration ratio of 2:1 iodine/compound of formula (II).

In certain embodiments, either C or D of formula (II) has a protected N-terminus and is reacted with iodine (I₂). In certain embodiments, either C or D of formula (II) has a protected N-terminus and is reacted with iodine at a concentration ratio of 1:1 iodine/compound of formula (II).

If the linear peptide is coupled to the resin, 2 equivalents of iodine could accelerate the reaction on the solid phase. The solid support makes it possible to work with excesses of reagents and solvents. Therefore, two equivalents are not critical with regard to side reactions.

In contrast, with the peptide in solution, only one equivalent of iodine is used to prevent side reactions.

In certain embodiments, the reaction step (a) is performed in a polar solvent. In certain embodiments, the reaction step (a) is performed in MeOH, DCM, NMP (N-Methyl-2-pyrrolidon) or DMF. In certain embodiments, the reaction step (a) is performed in a polar aprotic solvent. In certain embodiments, the reaction step (a) is performed in DMF. In certain embodiments, the reaction step (a) is performed under mild acidic conditions such as solvent mixtures containing TFA (low concentrations such as 1%), TFE (trifluoroethanol), AcOH or HFIP (hexafluoroisopropanol) in DCM.

In certain embodiments, iodine is used at a concentration of 1-4 mg/ml, particularly at 2 mg/ml. In certain embodiments, n is 3.

In certain embodiments, A is independently selected from a proteinogenic or non-proteinogenic α-amino acid in L- or D-conformation. In certain embodiments, A is independently selected from unsubstituted or hydroxyl-substituted Gly, Ala, Ile, Leu, Val, Pro, Phe, Lys, Arg, His, D-Pro, D-Ala, L-Propargyl-Gly, Aib (aminoisobutyric acid of formula

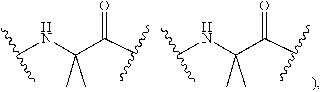

a photo amino acid (particularly Photo-Leu), an azide amino acid, an alkynyl amino acid.

Photo amino acids comprise a diazirine group of formula

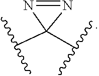 [SMILES: *C1(*)N=N1]

Examples for photo amino acids are photo-Leu, photo-Met, and photo-Phe.

Azide amino acids comprise an azido group (—N₃).

Alkynyl amino acids comprise an alkynyl group of formula

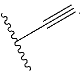 [SMILES: *C#C]

In certain embodiments, C and D are independently selected from a proteinogenic or non-proteinogenic α-amino acid in L- or D-conformation. In certain embodiments, C and D are independently selected from Gly, Ala, Ile, Leu, Val, Pro, Phe, Lys, Ser, Cys, Arg, His, Asp, Asn, Gln, Glu, Hyp, L-Pipecolinic acid (3105-95-1), L-Azetidine-2-carboxylic acid (2133-34-8), (S)-Indoline-2-carboxylic acid (79815-20-6), L-4-Thiazolidinecarboxylic acid (34592-47-7), trans-4-Hyp (of formula

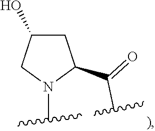 [SMILES: *C(=O)[C@@H]1C[C@@H](O)CN1*]

L-Propargyl-Gly, a hydroxylated amino acid, a photo amino acid (particularly photo-Pro or photo-Leu), an azide amino acid (particularly azido-Pro), an alkynyl amino acid (particularly alkynyl Pro), fluorinated Pro (particularly 4-F-Pro), amino-Pro (particularly 4-amino-Pro of formula).

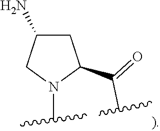 [SMILES: *C(=O)[C@@H]1C[C@@H](N)CN1*]

Hydroxylated amino acids comprise at least one OH-group.

In certain embodiments, the indole of Y is unsubstituted or substituted with one, two, three or four groups selected from hydroxyl, halogen, CN and a fluorinated carbon (CF₃, CHF₂, or CH₂F). In certain embodiments, the indole of Y is unsubstituted or substituted with one hydroxyl or halogen group.

If Y comprises a hydroxyl group, this group may be deprotected or protected with a hydroxyl-protecting group, preferably protected.

In certain embodiments, the indole of Y is unsubstituted indole. In certain embodiments, indole is connected to the linker L¹ via its 3-position

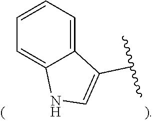

In certain embodiments, the indole is connected via its 2-position to the sulphur atom.

In certain embodiments, the resin is an acid labile resin. In certain embodiments, the resin is a 2-chlorotrityl resin, a rink amide resin, 1,3-dihydro-2H-pyran-2-yl-methoxymethyl resin (THP-resin) or a Wang resin.

In certain embodiments, the sulphur atom of Z is oxidized. In certain embodiments, the sulphur atom of Z is oxidized
i. using manganese ions, particularly the compound is reacted with a compound of formula (V)

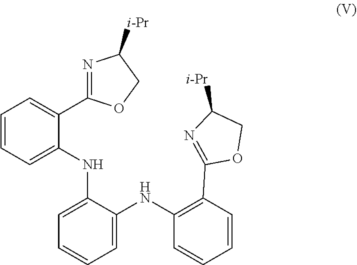 [SMILES: CC(C)[C@H]1COC(c2ccccc2Nc2ccccc2Nc2ccccc2C2=N[C@@H](C(C)C)CO2)=N1]

and with Mn(OTf₂ and H₂O₂,
ii. using PPO, dibenzyolperoxide, tert-butyl peroxybenzoate, or lauroyl peroxide; or mCPBA;
iii. using iodine and oxygen.

A second aspect of the invention relates to a method for preparation of a compound of formula (VI)

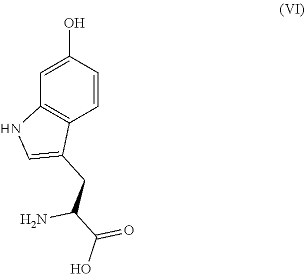

wherein a compound of formula (VII)

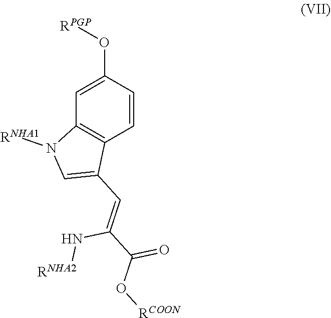

wherein
R^{NHA1} is an amino protecting group, particularly an amino protecting group cleavable under acidic or reductive conditions, more particularly Boc, Cbz, or triisopropylsilyl (TIPS), preferably Boc,
R^{NHA2} is an amino protecting group, particularly an amino protecting group cleavable under acidic or reductive conditions, more particularly Boc or Cbz, most particularly Boc,
R^{PGP} is a protecting group for phenolic OH groups, particularly a phenolic OH-protecting group cleavable under basic or reductive conditions, more particularly cleavable under reductive conditions, more particularly R^{PGP} is benzyl or acetyl, most particularly R^{PGP} is benzyl;
R^{COON} is a carboxyl-protecting group, particularly a carboxyl-protecting group cleavable under basic conditions, more particularly methyl, ethyl, benzyl, or tert-butyl, most particularly methyl,

is reacted with H₂, an organometallic rhodium or ruthenium complex and organophosphorus ligand, particularly with H₂, an organometallic rhodium complex and organophosphorus ligand, more particularly with H₂ and Bis(1,5-cyclooctadiene) rhodium (I)⁺, Bis(2,2′-bipyridine) rhodium (I)⁺ or Bis(norbornadiene) rhodium (I)⁺ and an anion selected from trifluoromethanesulfonate (CF₃SO₃—), hexafluorophosphate (PF₆⁻), chloride, and tetrafluoroborate (BF₄) and an organophosphorus ligand selected from (R)-MonoPhos [(R)-(−)-(3,5-Dioxa-4-phosphacyclohepta[2,1-a:3,4-a′ ]dinaphthalen-4-yl)dimethylamine] or (1R,1′ R,2S,2′S)-DuanPhos, [(1R,1′ R,2S,2′S)-2,2′-Di-tert-butyl-2,3,2′,3′-tetrahydro-1H,1′ H-(1,1) biisophos phindolyl] or (R,R)-DIPAMP, [(R,R)-1,2-Bis[(2-methoxyphenyl) (phenyl phosphino)]ethane], most particularly with H₂, Rh(COD)₂BF₄ (Bis(1,5-cyclooctadiene) rhodium (I)tetrafluoroborate) and (R)-MonoPhos, [(R)-(−)-(3,5-Dioxa-4-phosphacyclohepta[2,1-a:3,4-a′]dinaphthalen-4-yl) dimethylamine] in a reaction step (a) and the compound is reacted with a deprotection agent removing R^{NHA1}, R^{NHA2}, R^{PGP}, and R^{COON} to yield the compound characterized by (VI).

For R^{NHA2} acid labile and reduction labile groups are suitable since an alkali labile group would be cleaved during hydrolysis of R^{COON} group.

For R^{PGP}, an acid labile group would be cleaved during the Vilsmeier-Haack reaction. In contrast, reduction labile groups are suitable. An alkali labile group works if the R^{COON} group is reduction labile.

In certain embodiments, a compound of formula (VIII)

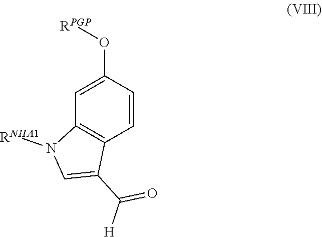

wherein
R^{NHA1} and R^{PGP} have the same meanings as described in the second aspect, is reacted with a protected 2-phosphonoglycine-methyl dimethyl ester under basic conditions, particularly with Boc-2-phosphonoglycine-methyl dimethyl ester, Cbz-2-phosphonoglycine-benzyl dimethyl ester or Cbz-2-phosphonoglycine-methyl dimethyl ester under basic conditions, more particularly the base is selected from DBU (1,8-Diazabicyclo(5.4.0)undec-7-ene) and tetramethylguanidine (CAS 80-70-6) in a reaction step (b) to yield the compound characterized by (VII).

A third aspect of the invention relates to a compound of formula (IIIa) and (IIIb)

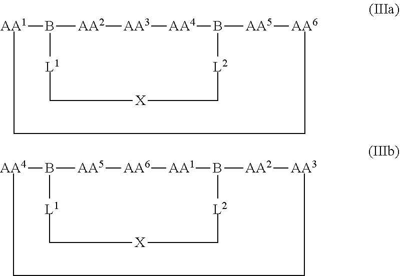

or a compound of formula (IVa), (IVb), (IVc), (IVd), (IVe), (IVf), (IVg), or (IVh)

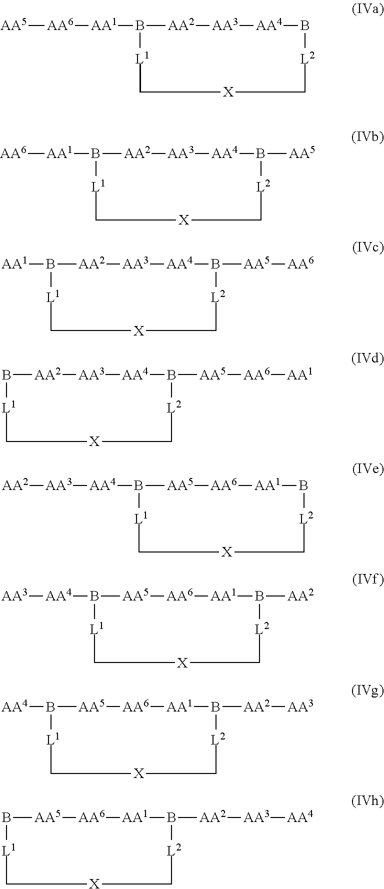

wherein
X is either of formula Q-R in formula [(IVa), (IVb), (IVc) and (IVd)], or R-Q in formula [(IVe), (IVf), (IVg) and (IVh)],
Q is unsubstituted or CF₃—, alcohol-, alkyl-, O-alkyl-, hydroxyl-, and/or halogen-substituted imidazole or indole, more particularly Q is unsubstituted or alkyl-, O-alkyl-, hydroxyl-, and/or halogen-substituted indole, most particularly Q is selected from unsubstituted indole, 6-OH-indole, 5-OH-indole, 5-F-indole, 5-Me-indole, 5-OMe-indole, 4-F-indole, and 5-Br-indole;
R is S, SO, or SO₂;
L¹ is an unsubstituted or Me-substituted C₁-C₂ alkyl linker, particularly L¹ is an unsubstituted C₁ alkyl linker;
L² is an unsubstituted or Me-substituted C₁-C₂ alkyl linker;
B is an α-amino acid backbone;
AA¹ is selected from DHIle (dihydroxy-isoleucin), Ile, DHLeu (dihydroxy-leucin), HVal (hydroxy-valin), HIle (5-hydroxy-isoleucin or 4-hydroxyisoleucine), Gly, D-Pro, L-Pro, D-Ala, Aib (2-aminoisobutyric acid), and Ala, particularly is selected from Gly and D-Pro, DHIle (dihydroxy-isoleucin), Ile, DHLeu (dihydroxy-leucin), HVal (hydroxy-valin), HIle (5-hydroxy-isoleucin or 4-hydroxyisoleucine);
AA² is selected from Gly, D-Pro, D-Ala, Aib (2-aminoisobutyric acid), Asn, Asp, Ser, Cys, Arg, Lys, Gln, Glu, and L-Propargyl-Gly, particularly is selected from Asn, Asp, Gln, Glu; and Ala, Gly and D-Pro;
AA³ is selected from Ile, Leu, Val, PhotoLeu, and L-Propargyl-Gly, L-propargy-Gly Pro, Gly, Aib, trans-4-Hyp, Hyp, 4-F-Pro, 4-NH₂—Pro, Photo-Pro, L-Pipecolinic acid, L-azetidine-2-carboxylic acid, (S)-Indoline-2-carboxylic acid, L-4-Thiazolidinecarboxylic acid, Tle, particularly is selected from trans-4-Hyp, 4-F-Pro, and 4-NH₂—Pro; most particular from Ile
AA⁴ is selected from Gly, D-Pro, D-Ala, Aib (2-aminoisobutyric acid), Asn, Asp, Ser, Cys, Arg, Lys, Gln, Glu, and L-Propargyl-Gly, Sar, Acc, particularly is selected from Asn, Asp, Gln, Glu; and Ala, most particularly AA² is selected from Gly and D-Pro;
AA⁵ is selected from Asn, Asp, Ser, Cys, Arg, Lys, Gln, Glu, and L-Propargyl-Gly, D-Pro, D-Ala, Aib (2-aminoisobutyric acid), and Ala, particularly is selected from Gly and D-Pro, Asn, Asp, Gln, and Glu;
AA⁶ is selected from Pro, Gly, Aib, trans-4-Hyp, Hyp, 4-F-Pro, 4-NH₂—Pro, Photo-Pro, L-Pipecolinic acid, L-azetidine-2-carboxylic acid, (S)-Indoline-2-carboxylic acid, L-4-Thiazolidinecarboxylic acid, Ile, Leu, Val, PhotoLeu, and L-Propargyl-Gly, particularly is selected from Ile and L-propargyl-Gly, trans-4-Hyp, 4-F-Pro, 4-F₂-Pro and 4-NH₂—Pro;
particularly with the proviso that in formula (IIIa) either the N-terminus of AA¹ is coupled via an amid bond to the C-terminus of AA⁶; and in formula (IIIb) the N or C-terminus of AA⁴AA¹ is coupled via an amide bond to the N-terminus of AA⁶; AA³
and that in formula (IVa), (IVb), (IVc), (IVd), (IVe), (IVf), (IVg), or (IVh) the C-terminal amino acid is optionally connected to a resin;
particularly with the proviso that the direction from left to right is either from N-terminus to C-terminus or from C-terminus to N-terminus.

In a particular embodiment of the compound according to the present invention, said compound is not composed of the following combinations:
Q is unsubstituted or alkyl-, O-alkyl-, hydroxyl-, and/or halogen-substituted indole;
R is S, SO, or SO₂;
L¹ is CH₂;
L² is CH₂;
AA¹ is selected from DHIle (dihydroxy-isoleucin), Ile, and HIle (hydroxy-isoleucin);
AA² is Gly;
AA³ is Ile;
AA⁴ is Gly;
AA⁵ is Asn or Asp; and
AA⁶ is Pro or Hyp (hydroxyproline);
or
Q is unsubstituted or alkyl-, O-alkyl-, hydroxyl-, and/or halogen-substituted indole;
R is S, SO, or SO₂;
L¹ is CH₂;
L² is CH₂;
AA¹ is DHIle or Ile;
one of AA² and AA⁴ is Gly and the other one is Ala, or both AA² and AA⁴ are Ala;
AA³ is Ile;
AA⁵ is Asn; and
AA⁶ is Hyp (hydroxyproline);
or
Q is unsubstituted or alkyl-, O-alkyl-, hydroxyl-, and/or halogen-substituted indole;
R is S, SO, or SO₂;
L¹ is CH₂;
L² is CH₂;
AA¹ is selected from DHLeu (dihydroxy-leucin), and HVal (hydroxy-valin);
AA² is Gly;
AA³ is Ile;
AA⁴ is Gly;
AA⁵ is Asn; and
AA⁶ is Hyp (hydroxyproline).

In certain embodiments, indole is connected to the linker L¹ via its 3-position. In certain embodiments, indole is connected to the sulphur atom via its 2-position.

In some embodiments, the present invention provides for compounds obtainable by the inventive method as disclosed herein.

In some embodiments, the compounds obtainable by the inventive method as disclosed above are used in the manufacture of antibody drug conjugates (ADCs).

According to one embodiment, the invention pertains to the use of a compound obtainable by the inventive method as disclosed herein in the manufacture of an antibody-drug conjugate wherein the compound is selected from the group of compounds comprising

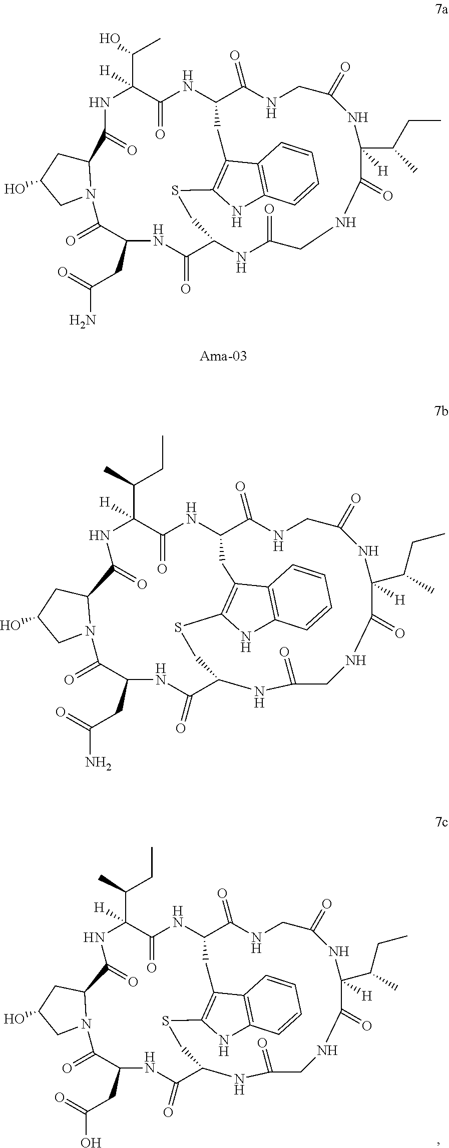 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)O)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(=O)O)C(=O)N1C[C@H](O)C[C@H]1C(=O)N[C@@H]([C@@H](C)CC)C(=O)N2]

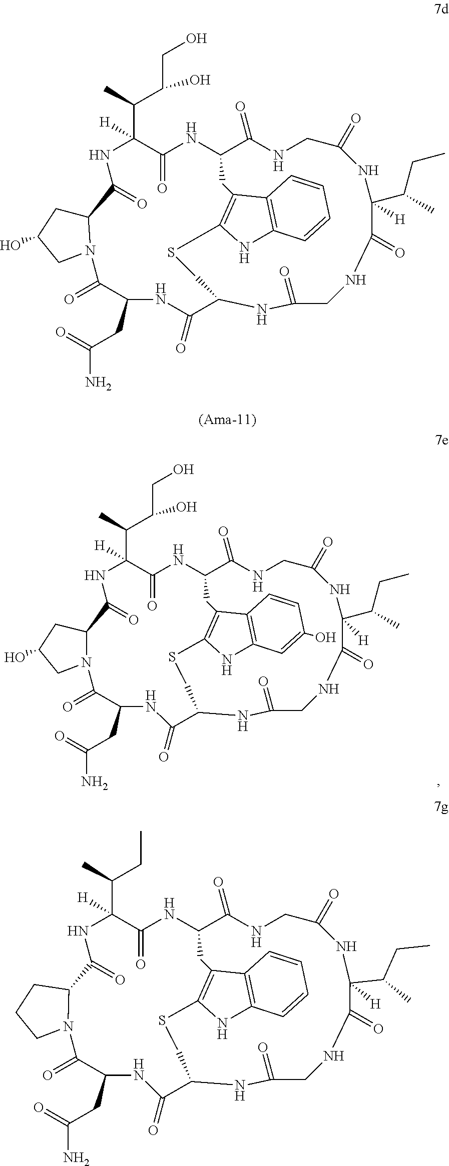 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4cc(O)ccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1CCC[C@@H]1C(=O)N[C@@H]([C@@H](C)CC)C(=O)N2]

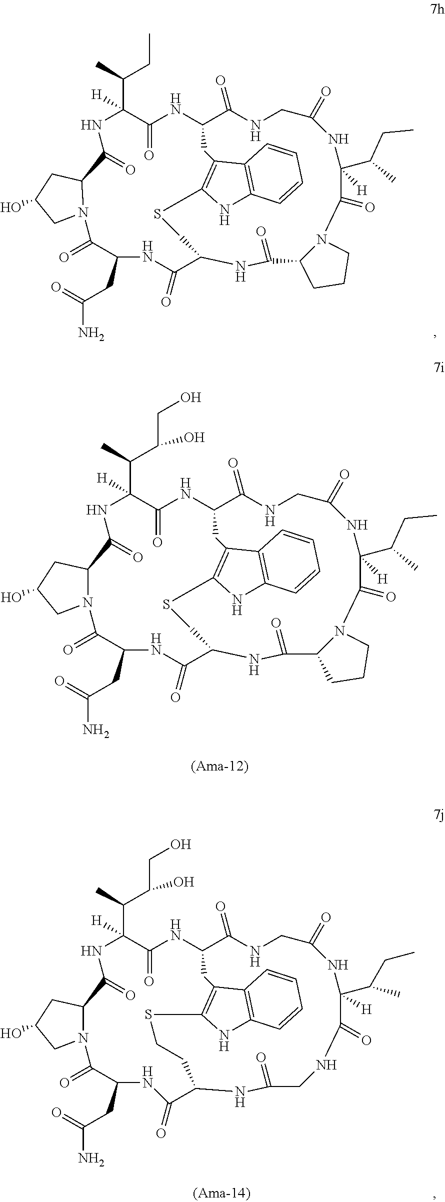

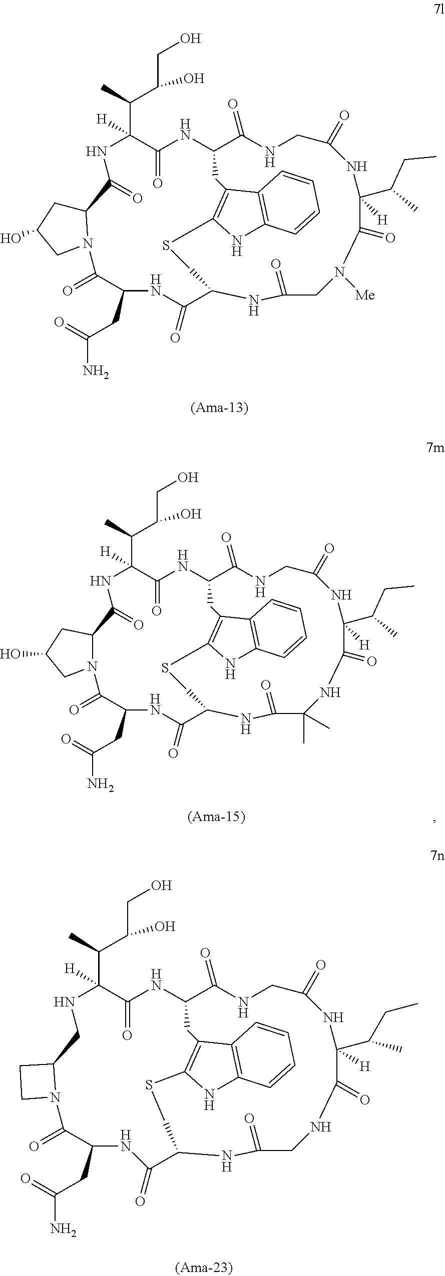 [SMILES: CC[C@H](C)[C@@H]1NC(=O)CNC(=O)[C@@H]2Cc3c([nH]c4ccccc34)SC[C@H](NC(=O)CN(C)C1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4ccccc34)SC[C@H](NC(=O)C(C)(C)NC1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1CC[C@H]1CN[C@@H]([C@@H](C)[C@@H](O)CO)C(=O)N2]

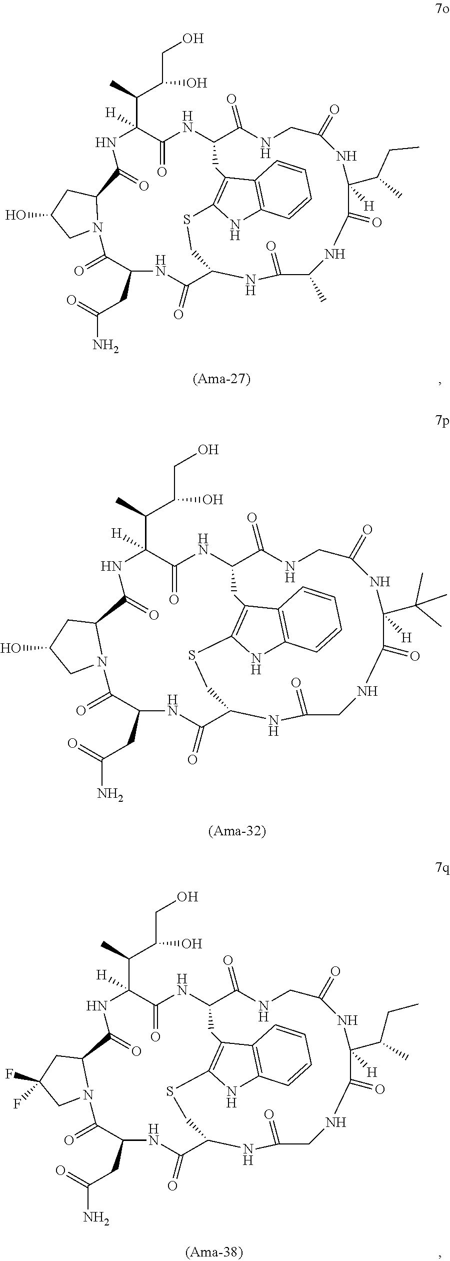

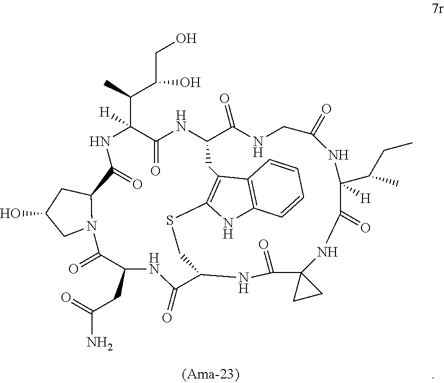 [SMILES: CC[C@H](C)[C@@H]1NC(=O)CNC(=O)[C@@H]2Cc3c([nH]c4ccccc34)SC[C@H](NC(=O)C3(CC3)NC1=O)C(=O)N[C@@H](CC(N)=O)C(=O)N1C[C@H](O)C[C@H]1C(=O)N[C@@H]([C@@H](C)[C@@H](O)CO)C(=O)N2]

According to one embodiment, the invention pertains to the use of the inventive compounds as disclosed above as ADC payloads. The term “payload” as used in the present invention refers to a to a biologically active cytotoxic (anticancer) drug, such as e.g. the compounds obtainable by the inventive method, e.g. compounds 7a-7r of the invention, which is conjugated to (i) an antibody, preferably a monoclonal antibody, (ii) an antigen-binding fragment thereof, preferably a variable domain (Fv), a Fab fragment or an F(ab)2 fragment, (iii) an antigen-binding derivative thereof, preferably a single-chain Fv (scFv), and (iv) an antibody-like protein.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or VH) and a heavy chain constant region (abbreviated as CH or CH). The heavy chain constant region is comprised of three domains, namely CH1, CH2 and CH3. Each light chain contains a light chain variable region (abbreviated as VL or VL) and a light chain constant region (abbreviated as CL or CL). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen-binding fragment” of the (monoclonal) antibody refers to one or more fragments of an antibody which retain the ability to specifically bind to its antigen in its native form. Examples of antigen binding portions of the antibody include a Fab fragment, a monovalent fragment consisting of the VL, VH, CL and CH1 domains, an F(ab′)2 fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR).

The antibody, or antibody fragment or antibody derivative thereof, according to the present invention can be a monoclonal antibody. As used herein, the term “monoclonal antibody” (“mAb”) refers to a preparation of antibody molecules of single binding specificity and affinity for a particular epitope, representing a homogenous antibody population, i.e., a homogeneous population consisting of a whole immunoglobulin, or a fragment or derivative thereof.

Preferably, such antibody is selected from the group consisting of IgG, IgD, IgE, IgA and/or IgM, or a fragment or derivative thereof, preferably the monoclonal antibody of the invention is of the IgG isotype, e.g. IgG1, or IgG4, more preferably of the IgG1 isotype.

As used herein, the term “fragment” or “antigen-binding fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)2.

As used herein, the term “derivative” or “antigen-binding derivative” shall refer to protein constructs being structurally different from, but still having some structural relationship to, the common antibody concept, e.g., scFv, Fab and/or F(ab)2, as well as bi-, tri- or higher specific antibody constructs, all of which have about the same target-binding specificity as the monoclonal antibodies of the invention.

Other antibody derivatives known to the skilled person are Diabodies, Camelid Antibodies, Domain Antibodies, bivalent homodimers with two chains consisting of scFvs, IgAs (two IgG structures joined by a J chain and a secretory component), shark antibodies (IgNAR), antibodies consisting of new world primate framework plus non-new world primate CDR, dimerised constructs comprising CH3+VL+VH, other scaffold protein formats comprising CDRs, and antibody conjugates (e.g., antibody, or fragments or derivatives thereof, linked to a drug, a toxin, a cytokine, an aptamer, a nucleic acid such as a desoxyribonucleic acid (DNA) or ribonucleic acid (RNA), a therapeutic polypeptide, a radioisotope or a label).

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein.

Antibody-like proteins include without limitation affilin proteins, affibodies, anti-calins, and designed ankyrin repeat proteins (Binz et al., 2005). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

According to one embodiment, the compounds of the invention as disclosed above may e.g. be coupled to any of (i) an antibody, preferably a monoclonal antibody, (ii) an antigen-binding fragment thereof, preferably a variable domain (Fv), a Fab fragment or an F(ab)2 fragment, (iii) an antigen-binding derivative thereof, preferably a single-chain Fv (scFv), and (iv) an antibody-like protein directly via a bond, or via a linker, preferably via a linker. The term “linker” as used in the context of the of the present invention refers to a structure that is connecting two components, each being attached to one end of the linker.

In particular embodiments, the linker increases the distance between two components and alleviates steric interference between these components, such as in the present case between the antibody and the compounds of the invention. In particular embodiments, the linker has a continuous chain of between 1 and 30 atoms (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 atoms) in its backbone, i.e. the length of the linker is defined as the shortest connection as measured by the number of atoms or bonds between the inventive compound moiety and (i) the antibody, preferably a monoclonal antibody, (ii) an antigen-binding fragment thereof, preferably a variable domain (Fv), a Fab fragment or an F(ab)2 fragment, (iii) an antigen-binding derivative thereof, preferably a single-chain Fv (scFv), or (iv) an antibody-like protein, wherein one side of the linker backbone has been reacted with the compound of the invention and, the other side is available for reaction, or has been reacted, with e.g. an antibody. In the context of the present invention, a linker is e.g. a C₁₋₂₀-alkylene, C₁₋₂₀-heteroalkylene, C₂₋₂₀-alkenylene, C₂₋₂₀-heteroalkenylene, C₂₋₂₀-alkynylene, C₂₋₂₀-heteroalkynylene, cycloalkylene, heterocycloalkylene, arylene, heteroarylene, aralkylene, or a heteroaralkylene group, optionally substituted. The linker may e.g. contain one or more structural elements such as carboxamide, ester, ether, thioether, disulfide, urea, thiourea, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the inventive compound and e.g. the antibody or antigen-binding fragment thereof, or any of the binding moieties disclosed above. To that end the linker comprises two groups, preferably at a proximal and distal end, which can (i) form a covalent bond to a group present in one of the components to be linked, preferably an activated group on a compound of the invention or the antibody, or antigen-binding fragment thereof or (ii) which is or can be activated to form a covalent bond with a group on an amatoxin, such as the compounds of the invention. Accordingly, it is preferred that chemical groups are at the distal and proximal end of the linker, which are the result of such a coupling reaction, e.g. an ester, an ether, a urethane, a peptide bond etc.

In particular embodiments, the linker may be a linear chain of between 1 and 20 atoms independently selected from C, O, N and S, particularly between 2 and 18 atoms, more particularly between 5 and 16 atoms, and even more particularly between 6 and 15 atoms. In particular embodiments, at least 60% of the atoms in the linear chain are C atoms. In particular embodiments, the atoms in the linear chain are linked by single bonds.

In particular embodiments. the linker may be an alkylene, heteroalkylene, alkenylene, heteroalkenylene, alkynylene, heteroalkynylene, cycloalkylene, heterocycloalkylene, arylene, heteroarylene, aralkylene, or a heteroaralkylene group, comprising from 1 to 4 heteroatoms selected from N, O, and S, wherein said linker is optionally substituted.

The term “alkylene” refers to a bivalent straight chain saturated hydrocarbon groups having from 1 to 20 carbon atoms, including groups having from 1 to 10 carbon atoms. In certain embodiments, alkylene groups may be lower alkylene groups. The term “lower alkylene” refers to alkylene groups having from 1 to 6 carbon atoms, and in certain embodiments from 1 to 5 or 1 to 4 carbon atoms. Examples of alkylene groups include, but are not limited to, methylene (—CH₂—), ethylene (—CH₂—CH₂—), n-propylene, n-butylene, n-pentylene, and n-hexylene.

The term “alkenylene” refers to bivalent straight chain groups having 2 to 20 carbon atoms, wherein at least one of the carbon-carbon bonds is a double bond, while other bonds may be single bonds or further double bonds. The term “alkynylene” herein refers to groups having 2 to 20 carbon atoms, wherein at least one of the carbon-carbon bonds is a triple bond, while other bonds may be single, double or further triple bonds. Examples of alkenylene groups include ethenylene (—CH═CH—), 1-propenylene, 2-propenylene, 1-butenylene, 2-butenylene, 3-butenylene, and the like. Examples of alkynylene groups include ethynylene, 1-propynylene, 2-propynylene, and so forth.

As used herein, “cycloalkylene” is intended to refer to a bivalent ring being part of any stable monocyclic or polycyclic system, where such ring has between 3 and 12 carbon atoms, but no heteroatom, and where such ring is fully saturated, and the term “cycloalkenylene” is intended to refer to a bivalent ring being part of any stable monocyclic or polycyclic system, where such ring has between 3 and 12 carbon atoms, but no heteroatom, and where such ring is at least partially unsaturated (but excluding any arylene ring). Examples of cycloalkylenes include, but are not limited to, cyclopropylene, cyclobutylene, cyclopentylene, cyclohexylene, and cycloheptylene. Examples of cycloalkenylenes include, but are not limited to, cyclopentenylene and cyclohexenylene.

As used herein, the terms “heterocycloalkylene” and “heterocycloalkenylene” are intended to refer to a bivalent ring being part of any stable monocyclic or polycyclic ring system, where such ring has between 3 and about 12 atoms, and where such ring consists of carbon atoms and at least one heteroatom, particularly at least one heteroatom independently selected from the group consisting of N, O and S, with heterocycloalkylene referring to such a ring that is fully saturated, and heterocycloalkenylene referring to a ring that is at least partially unsaturated (but excluding any arylene or heteroarylene ring).

The term “arylene” is intended to mean a bivalent ring or ring system being part of any stable monocyclic or polycyclic system, where such ring or ring system has between 3 and 20 carbon atoms, but has no heteroatom, which ring or ring system consists of an aromatic moiety as defined by the “4n+2” π electron rule, including phenylene.

As used herein, the term “heteroarylene” refers to a bivalent ring or ring system being part of any stable mono- or polycyclic system, where such ring or ring system has between 3 and 20 atoms, which ring or ring system consists of an aromatic moiety as defined by the “4n+2” π electron rule and contains carbon atoms and one or more nitrogen, sulfur, and/or oxygen heteroatoms.

In the context of the present invention, the term “substituted” is intended to indicate that one or more hydrogens present in the backbone of a linker is replaced with a selection from the indicated group(s), provided that the indicated atom's normal valency, or that of the appropriate atom of the group that is substituted, is not exceeded, and that the substitution results in a stable compound. The term “optionally substituted” is intended to mean that the linker is either unsubstituted or substituted, as defined herein, with one or more substituents, as defined herein. When a substituent is a keto (or oxo, i.e. ═O) group, a thio or imino group or the like, then two hydrogens on the linker backbone atom are replaced. Exemplary substituents include, for example, alkyl, alkenyl, alkynyl, cycloalkyl, heterocycloalkyl, aryl, heteroaryl, aralkyl, heteroaralkyl, acyl, aroyl, heteroaroyl, carboxyl, alkoxy, aryloxy, acyloxy, aroyloxy, heteroaroyloxy, alkoxycarbonyl, halogen, (thio)ester, cyano, phosphoryl, amino, imino, (thio)amido, sulfhydryl, alkylthio, acylthio, sulfonyl, a sulfate, a sulfonate, a sulfamoyl, a sulfonamido, nitro, azido, haloalkyl, including perfluoroalkyl (such as trifluoromethyl), haloalkoxy, alkylsulfanyl, alkylsulfinyl, alkylsulfonyl, alkylsulfonylamino, arylsulfonoamino, phosphoryl, phosphate, phosphonate, phosphinate, alkylcarboxy, alkylcarboxyamide, oxo, hydroxy, mercapto, amino (optionally mono- or di-substituted, e.g. by alkyl, aryl, or heteroaryl), imino, carboxamide, carbamoyl (optionally mono- or di-substituted, e.g. by alkyl, aryl, or heteroaryl), amidino, aminosulfonyl, acylamino, aroylamino, (thio)ureido, (arylthio)ureido, alkyl(thio)ureido, cycloalkyl(thio)ureido, aryloxy, aralkoxy, or —O(CH2)n-OH, —O(CH2)n-NH2, —O(CH2)nCOOH, —(CH2)nCOOH, —C(O)O(CH2)nR, —(CH2)nN(H)C(O)OR, or —N(R)S(O)2R wherein n is 1-4 and R is independently selected from hydrogen, -alkyl, -alkenyl, -alkynyl, -cycloalkyl, -cycloalkenyl, —(C-linked-heterocycloalkyl), —(C-linked-heterocycloalkenyl), -aryl, and -heteroaryl, with multiple degrees of substitution being allowed. It will be understood by those skilled in the art that substituents, such as heterocycloalkyl, aryl, heteroaryl, alkyl, etc., or functional groups such as —OH, —NHR etc., can themselves be substituted, if appropriate. It will also be understood by those skilled in the art that the substituted moieties themselves can be substituted as well when appropriate.

In particular embodiments, the linker L comprises a moiety selected from one of the following moieties: a disulfide (—S—S—), an ether (—O—), a thioether (—S—), an amine (—NH—), an ester (—O—C(═O)— or —C(═O)—O—), a carboxamide (—NH—C(═O)— or —C(═O)—NH—), a urethane (—NH—C(═O)—O— or —O—C(═O)—NH—), and a urea moiety (—NH—C(═O)—NH—).

In particular embodiments of the present invention, the linker L comprises a number of m groups selected from the list of: alkylene, alkenylene, alkynylene, cycloalkylene, heteroalkylene, heteroalkenylene, heteroalkynylene, heterocycloalkylene, arylene, heteroarylene, aralkylene, and a heteroaralkylene group, wherein each group may optionally be independently substituted, the linker further comprises a number of n moieties independently selected from one of the following moieties: a disulfide (—S—S—), an ether (—O—), a thioether (—S—), an amine (—NH—), an ester (—O—C(═O)— or —C(═O)—O—), a carboxamide (—NH—C(═O)— or —C(═O)—NH—), a urethane (—NH—C(═O)—O— or —O—C(═O)—NH—), and a urea moiety (—NH—C(═O)—NH—), wherein m=n+1. In particular embodiments, m is 2 and n is 1, or m is 3 and n is 2. In particular embodiments, the linker comprises 2 or 3 unsubstituted alkylene groups, and 1 or 2, respectively, disulfide, ether, thioether, amine, ester, carboxamide, urethane or urea moieties linking the unsubstituted alkylene groups.

In particular embodiments, the C atoms in the linear chain are independently part of optionally substituted methylene groups (—CH2-). In particular such embodiments, the optional substituents are independently selected from halogen and C1-6-alkyl, particularly methyl.

Said linker may e.g. be conjugated to the hydroxyprolyl-residue (Hyp) of the compounds of the invention as disclosed herein, or to the DHIle (dihydroxy-isoleucin) residue of the inventive compounds. Conjugation of a linker to a Hyp residue of the inventive compounds may e.g. be done according to the methods as disclosed in EP3735987 A1 the content of which is incorporated by reference, conjugation of a linker to DHIle (dihydroxy-isoleucin) of the inventive compounds may be done according to the methods disclosed in WO2020234461 A1 the content of which is incorporated by reference.

According to some embodiments, the linker conjugated to the inventive compounds as disclosed herein can be a non-cleavable (stable) or a cleavable linker. In the context of the present invention, the term “stable linker” refers to a linker that is stable (i) in the presence of enzymes, particularly of lysosomal peptidases, such as Cathepsin B, and (ii) in an intracellular reducing environment.

According to one embodiment, the stable linker does not contain (i) an enzyme-cleavable substructure, particularly no dipeptide sequence cleavable by Cathepsin B), and/or (ii) a disulfide group. In particular such embodiments, the linker has a length of up to 12 atoms, particularly from 2 to 10, more particularly from 4 to 9, and most particularly from 6 to 8 atoms.

In the context of the present invention a “cleavable linker” is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments. An enzymatically cleavable moiety according to the invention may also be referred to as “cleavable by an enzyme”. Enzymatic cleavage of the linker results in the intracellular release of the toxin cargo conjugated to the targeting moiety or antibody as disclosed herein, or a metabolite thereof after internalization (see Dubowchik et al., Bioconjug Chem. 13 (2002) 855-69).

Said cleavable linker can be selected from the group consisting of an enzymatically cleavable linker, preferably a protease-cleavable linker, and a chemically cleavable linker, preferably a linker comprising a disulfide bridge.

According to preferred embodiments of the present invention, the cleavage site is an enzymatically cleavable moiety comprising two or more amino acids. Preferably, said enzymatically cleavable moiety comprises a valine-alanine (Val-Ala), valine-citrulline (Val-Cit), valine-lysine (Val-Lys), valine-arginine (Val-Arg) dipeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands co-ordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harboring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In particular embodiments of the present invention, the cleavable site is cleavable by at least one agent selected from the group consisting of Cathepsin A or B, matrix metalloproteinases (MMPs), elastases, β-glucuronidase and β-galactosidase, preferably Cathepsin B.

In particularly preferred embodiments, the enzymatically cleavable linker according to the invention comprises a dipeptide selected from Phe-Lys, Val-Lys, Phe-Ala, Val-Ala, Phe-Cit and Val-Cit, particularly wherein the cleavable linker further comprises a β-aminobenzyl (PAB) spacer between the dipeptides and a compound of the invention obtainable by the inventive method, such as e.g. one of the compounds 7a, 7b, 7c, 7d, 7e, 7g, 7h, 7i, 7j, 7l, 7m, 7n, 7o, 7p, 7q, 7r as disclosed above, whereby the PAB moiety is linked or conjugated to the compounds of the invention:

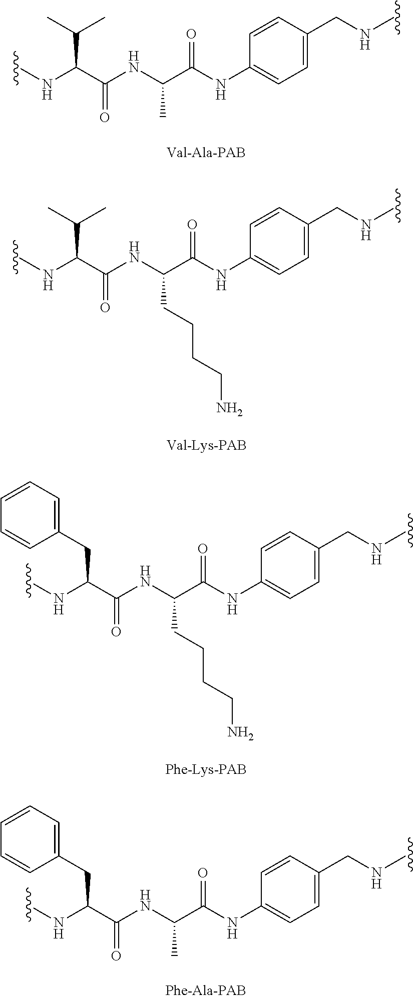

The conjugation of the linker-compound conjugates according to the invention as disclosed herein to an antibody, preferably a monoclonal antibody, an antigen-binding fragment thereof such as a variable domain (Fv), a Fab fragment or an F(ab)2 fragment, or an antigen-binding derivative thereof may be done by conjugation to reactive lysine or cysteine residues (see e.g. Jain et al. Pharm Res (2015) 32:3526-3540), preferably by conjugation to reactive cysteine residues to yield an antibody-drug conjugate (ADC).

According to some embodiments, the coupling or conjugation of the inventive compounds to reactive lysine residues may be done using linkers comprising one of the following reactive groups 1) SPDB disulfide, 2) MCC (maleimidomethyl cyclohexane-1-carboxylate), 3) sulfo-SPDB which adds a charged polar group and 4) hydrazone. Corresponding conjugation may e.g. be done as described in Peeters et al. J Immunol Methods. 1989 Jun. 2; 120(1):133-43, or Carlsson et al. Biochem J. 1978; 173:723-37.

According to preferred embodiments, the linkers of the invention as disclosed above comprise a thiol reactive group, selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide, preferably a maleimide (malemidyl residue). The conjugation to reactive cysteine residues using a malemidyl-based conjugation may e.g. be done as disclosed in WO2016/142049.

According to one embodiment, the antibody-drug-conjugates as disclosed above comprise from about 1 to about 10, preferably from about 1, 2 to about 4, 5, 6, 7, 8 compounds of the invention as disclosed herein coupled via a linker as disclosed herein to reactive lysine residues or cysteine residues of the antibody.

According to a preferred embodiment, the linker-compound conjugates according to the invention, as disclosed above may e.g. be conjugated by means of cysteine conjugation via site-specific conjugation to cysteine engineered antibodies comprising a heavy chain 118Cys, or a heavy chain 256Cys according to the EU numbering system (Edelman et al., Proc. Natl. Acad. Sci. USA; 63 (1969)) as disclosed in WO2016/142049 A1 the content of which is incorporated herein by reference. The use of said cysteine-engineered antibodies may be particularly advantageous to obtain antibody-drug conjugates comprising the inventive compounds as disclosed herein which have a controlled drug-to-antibody ratio (DAR) of about 2 (e.g. one linker-compound conjugate per heavy chain of the antibody) which e.g. can result in a better therapeutic index of said ADCs compared to ADCs having a higher DAR, or ADC preparations which comprise a mixture of ADC species having a DAR of about 1 to about 6, 8, or 10. The term “therapeutic index” as used herein is a quantitative measurement of the relative safety of a drug and may e.g. be defined as TI=TD₅₀:ED₅₀, whereby TD50 refers to the toxic dose of drug in 50% of subjects and ED50 refers to the efficacious dose in 50% of subjects. Accordingly, a higher therapeutic index is preferable to a lower one in that for example a subject in need thereof would have to take a much higher dose of the inventive ADCs to reach the toxic threshold than the dose taken to elicit the therapeutic effect.

According to one embodiment, the cysteine-engineered antibodies as disclosed above may additionally comprise the amino acid substitutions L234A, L235A (according to the EU numbering system) in its Fc region, as disclosed in WO 2020/086776 A1 the content of which is incorporated herein by reference. The use of such engineered antibodies may e.g. be advantageous to further improve the therapeutic index (TI) of an antibody-drug conjugate comprising a compound obtainable by the inventive method, such as e.g. one of the compounds 7a, 7b, 7c, 7d, 7e, 7g, 7h, 7i, 7j, 7l, 7m, 7n, 7o, 7p, 7q, 7r as disclosed herein.

According to some embodiments, the invention pertains to antibody-drug conjugates comprising the inventive compounds obtainable by the inventive method or which comprise the inventive compounds as disclosed hereinabove. For example, an ADC according to the invention may comprise from about 1, 2, 4, to about 6, 8, 10, or about 2, 4, 6, 8, or about 2 to about 4, or about 2 inventive compounds 7a, 7b, 7c, 7d, 7e, 7g, 7h, 7i, 7j, 7l, 7m, 7n, 7o, 7p, 7q, 7r as disclosed herein which may e.g. be conjugated to the antibody via a stable or cleavable linker as disclosed herein, whereby a given ADC will not comprise a mixture of the inventive compounds as disclosed above.

Wherever alternatives for single separable features such as, for example, an amino acid or an oxidation method are laid out herein as “embodiments”, it is to be understood that such alternatives may be combined freely to form discrete embodiments of the invention disclosed herein. Thus, any of the alternative embodiments for an amino acid may be combined with any of the alternative embodiments of an oxidation method mentioned herein.

The invention is further illustrated by the following examples and figures, from which further embodiments and advantages can be drawn. These examples are meant to illustrate the invention but not to limit its scope.

### Examples

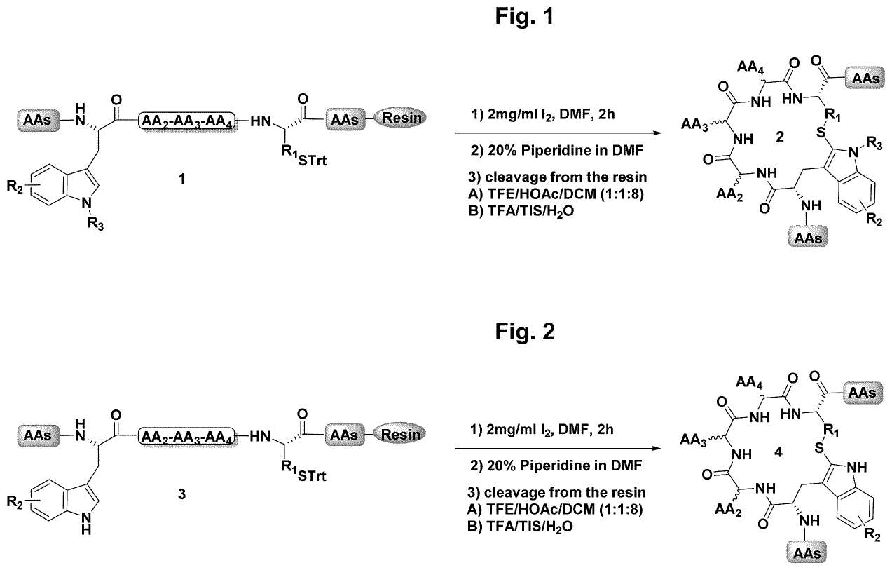

TABLE 1
Amino acid sequences of peptides bound to
solid support cyclized by iodine. The
isolated yield was calculated after
cleavage from the resin and purification
by preparative HPLC (see FIG. 1).

| | Peptide bound to | | |
|---|---|---|---|
| | the resin | | Isolated |
| Entry | (amino acid sequence) | Compound | yield |
| 1 | Fmoc-Trp-Gly-Ile-Gly- | 2a | 20% |
| | Cys(Trt)-O-resin | | |
| 2 | Fmoc-D-Trp-Gly-D-Ile- | 2b | 18% |
| | Gly-D-Cys(Trt)-O-resin | | |
| 3 | Fmoc-D-Trp-Gly-Ile- | 2c | 15% |
| | Gly-Cys(Trt)-O-resin | | |
| 4 | Fmoc-Trp-Gly-Tle-Gly- | 2d | 18% |
| | Cys(Trt)-O-resin | | |
| 5 | Fmoc-Trp-Gly-Phe-Gly- | 2e | 15% |
| | Cys(Trt)-O-resin | | |
| 6 | Fmoc-Trp-Gly-Glu(O^{t}Bu)- | 2f | 28% |
| | Gly-Cys(Trt)-O-resin | | |
| 7 | Fmoc-Trp-Gly-Trp-Gly- | 2g | 14% |
| | Cys(Trt)-O-resin | | |
| 8 | Fmoc-Trp-Gly-Lys(Boc)- | 2h | 29% |
| | Gly-Cys(Trt)-O-resin | | |
| 9 | Fmoc-Trp(5-OMe)-Gly- | 2i | 13% |
| | Ile-Gly-Cys(Trt)-O-resin | | |
| 10 | Fmoc-Trp(5-OTBS)-Gly- | 2j | 15% |
| | Ile-Gly-Cys(Trt)-O-resin | | |
| 11 | Teoc-Trp(6-OBn)-Gly- | 2k | 16% |
| | Ile-Gly-Cys(Trt)-O-resin | | |
| 12 | Fmoc-Trp(4-F)-Gly-Ile- | 2l | 12% |
| | Gly-Cys(Trt)-O-resin | | |
| 13 | Fmoc-Trp-Gly-Ile-Gly- | 2m | 18% |
| | HomoCys(Trt)-O-resin | | |
| 14 | Fmoc-Trp(Me)-Gly-Ile- | 2n | 15% |
| | Gly-Cys(Trt)-O-resin | | |
| 15 | Fmoc-Trp-Gly-Ile-D-Pro- | 2o | 20% |
| | Cys(Trt)-O-resin | | |
| 16 | Fmoc-Trp-Trp-Gly-Ile- | 2p | 13% |
| | Gly-Cys(Trt)-O-resin | | |
| 17 | Fmoc-Ile-Trp-Gly-Cys | 2q | 38% |
| | (S^{t}Bu)-Gly-Cys(Trt)- | | |
| | Asn(Trt)-O-resin | | |
| 18 | Fmoc-Ile-Trp-Gly-Ile- | 2r | 50% |
| | Gly-Cys(Trt)-Asn(Trt)- | | |
| | O-resin | | |
| 19 | Fmoc-Ile-Trp-Ile-Gly- | 2s | 40% |
| | Ile-Cys(Trt)-Asn(Trt)-O- | | |
| | resin | | |

Analytical Data:

Compound 2a: HPLC-MS: Retention time Rₜ=8.26 min (Gradient A); HRMS (ESI): m/z calculated: C₃₉H₄₃N₆O₈S⁺ [M+Na]⁺ 755.2858, found 777.2681. Yield (purified after RP-HPLC): 22 mg.

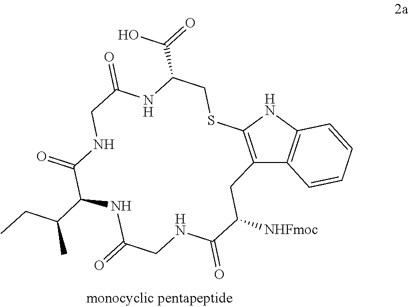

Compound 2b: HPLC-MS: Retention time Rₜ=8.50 min (Gradient A); HRMS (ESI): m/z calculated: C₃₉H₄₃N₆O₈S⁺ [M+H]⁺ 755.2858, found 755.2864. Yield (purified after RP-HPLC): 20 mg.

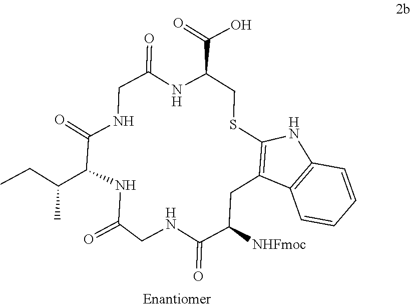

Compound 2c: HPLC-MS: Retention time Rₜ=8.59 min (Gradient A); HRMS (ESI): m/z calculated: C₃₉H₄₃N₆O₈S⁺ [M+H]⁺ 755.2858, found 755.2861. Yield (purified after RP-HPLC): 16.5 mg.

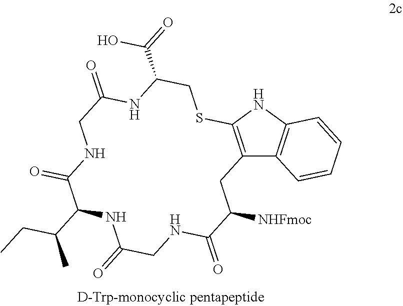

Compound 2d: HPLC-MS: Retention time Rₜ=8.53 min (Gradient A); HRMS (ESI): m/z calculated: C₃₉H₄₃N₆O₈S⁺ [M+H]⁺ 755.2858, found 755.2863. Yield (purified after RP-HPLC): 20 mg.

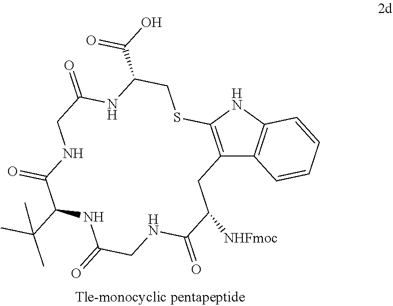

Compound 2e: HPLC-MS: Retention time Rₜ=8.74 min (Gradient A); HRMS (ESI): m/z calculated: C₄₂H₄₁N₆O₈S⁺ [M+H]⁺ 789.2701, found 789.2706. Yield (purified after RP-HPLC): 17 mg.

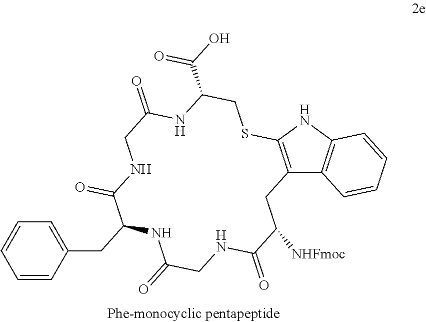

Compound 2f: HPLC-MS: Retention time Rₜ=8.86 min (Gradient A); HRMS (ESI): m/z calculated: C₄₂H₄₇N₆O₁₀S⁺ [M+H]⁺ 827.3069, found 827.3073. Yield (purified after RP-HPLC): 34 mg.

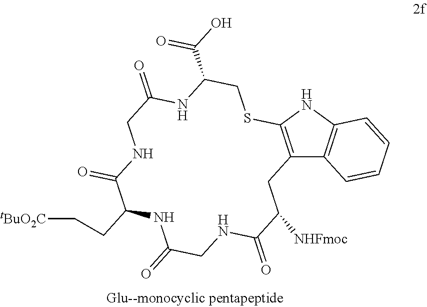

Compound 2g: HPLC-MS: Retention time Rₜ=8.67 min (Gradient A); HRMS (ESI): m/z calculated: C₄₄H₄₂N₆O₈S⁺ [M+H]⁺ 828.2610, found 828.2614. Yield (purified after RP-HPLC): 17 mg.

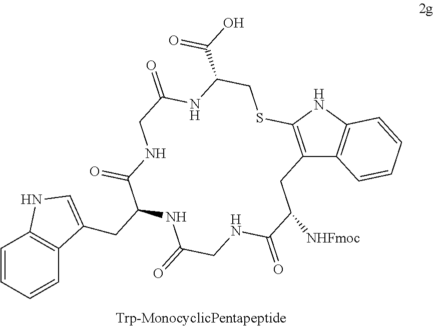

Compound 2h: HPLC-MS: Retention time Rₜ=8.82 min (Gradient A); HRMS (ESI): m/z calculated: C₄₄H₅₂N₇O₁₀S⁺ [M+H]⁺ 870.3491, found 870.3520. Yield (purified after RP-HPLC): 37 mg.

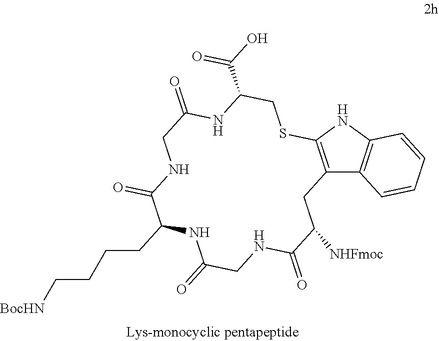

Compound 2i: HPLC-MS: Retention time Rₜ=8.37 min (Gradient A); HRMS (ESI): m/z calculated: C₄₀H₄₅N₆O₉S⁺ [M+H]⁺ 785.2963, found 785.2977. Yield (purified after RP-HPLC): 15 mg.

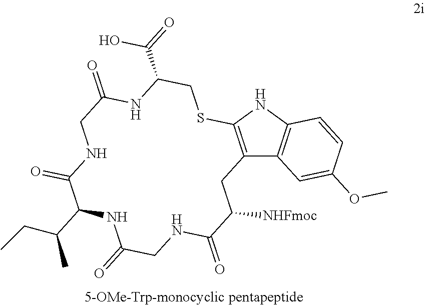

Compound 2j: HPLC-MS: Retention time Rₜ=7.54 min (Gradient A); HRMS (ESI): m/z calculated: C₃₉H₄₃N₆O₉S⁺ [M+H]⁺ 771.2807, found 771.2817. Yield (purified after RP-HPLC): 17 mg.

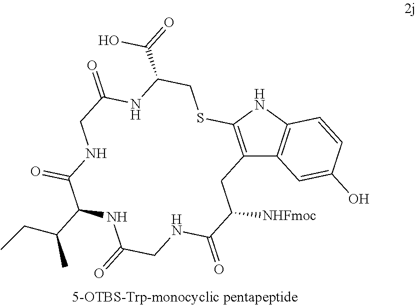

Compound 2k: HPLC-MS: Retention time Rₜ=9.20 min (Gradient A); HRMS (ESI): m/z calculated: C₃₇H₅₁N₆O₉SSi⁺ [M+H]⁺ 783.3202, found 783.3230. Yield (purified after RP-HPLC): 18 mg.

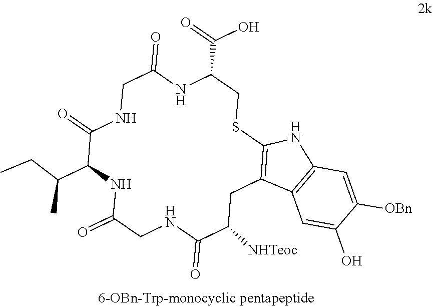

Compound 2l: HPLC-MS: Retention time Rₜ=8.54 min (Gradient A); HRMS (ESI): m/z calculated: C₃₉H₄₂FN₆O₈S⁺ [M+H]⁺ 773.2763, found 773.2770. Yield (purified after RP-HPLC): 14 mg.

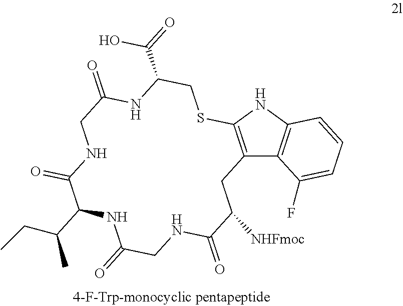

Compound 2m: HPLC-MS: Retention time Rₜ=8.80 min (Gradient A); HRMS (ESI): m/z calculated: C₄₀H₄₅N₆O₈S⁺ [M+H]⁺ 769.3014, found 769.3020. Yield (purified after RP-HPLC): 20 mg.

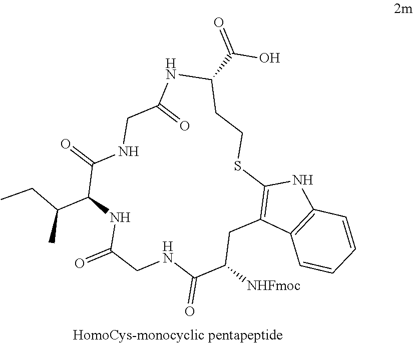

Compound 2n: HPLC-MS: Retention time Rₜ=7.76 min (Gradient A); HRMS (ESI): m/z calculated: C₄₀H₄₄N₆NaO₈S⁺ [M+Na]⁺791.2834, found 791.7598. Yield (purified after RP-HPLC): 17 mg.

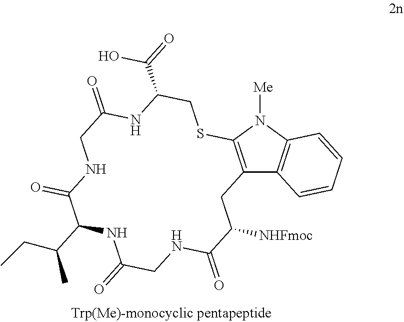

Compound 2o: HPLC-MS: Retention time Rₜ=9.04 min (Gradient A); HRMS (ESI): m/z calculated: C₄₂H₄₇N₆O₈S⁺ [M+H]⁺ 795.3171, found 795.3177. Yield (purified after RP-HPLC): 24 mg.

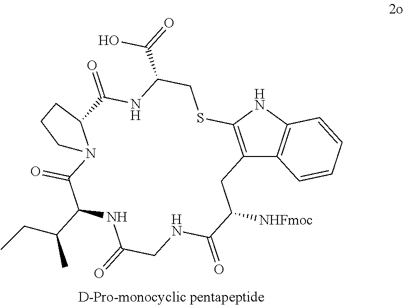

Compound 2p: HPLC-MS: Retention time Rₜ=8.68 min (Gradient A); HRMS (ESI): m/z calculated: C₅₁H₅₇N₈O₉S⁺ [M+H]⁺ 957.3964, found 957.3619. Yield (purified after RP-HPLC): 37 mg.

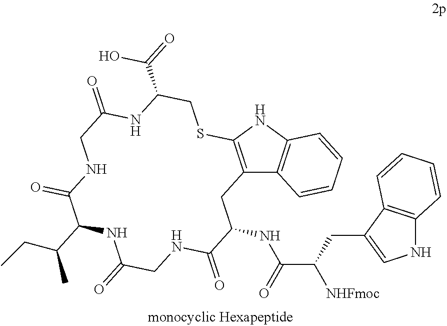

Compound 2q: HPLC-MS: Retention time Rₜ=11.55 min (Gradient A); HRMS (ESI): m/z calculated: C₆₉H₇₆N₉O₁₁S₃⁺ [M+H]⁺ 1302.4821, found 1302.4819. Yield (purified after RP-HPLC): 74 mg.

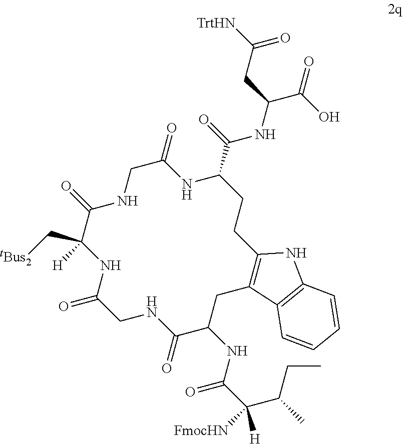

Compound 2r: HPLC-MS: Retention time Rₜ=8.16 min (Gradient C); HRMS (ESI): m/z calculated: C₆₈H₇₄N₉O₁₁S⁺ [M+H]⁺ 1224.5223, found 1224.5250. Yield (purified after RP-HPLC): 91 mg.

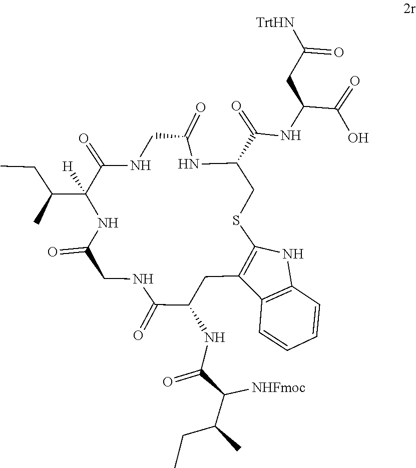

Compound 2s: HPLC-MS: Retention time Rₜ=10.79 min (Gradient C); HRMS (ESI): m/z calculated: C₇₂H₈₂N₉O₁₁S⁺ [M+H]⁺ 1280.5849, found 1280.5831. Yield (purified after RP-HPLC): 76 mg.

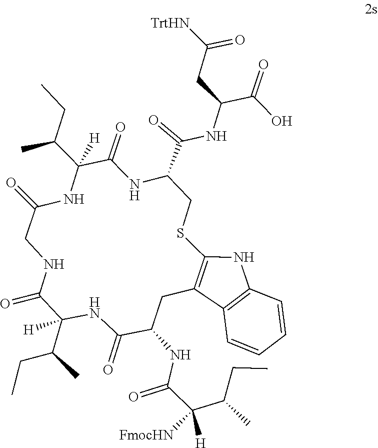

TABLE 2
Amino acid sequences of peptides bound to solid support cyclized by iodine. The
isolated yield was calculated after removal of the Fmoc group, cleavage from the resin and
purification by preparative HPLC. The table shows examples where the cyclization took place
via the B-ring of amanitin (N-terminal Trp and C-terminal Cys) (see FIG. 2).

| | Peptide bound to the resin | | Isolated |
|---|---|---|---|
| Entry | (amino acid sequence) | Compound | yield |
| 1 | Fmoc-Trp(6OBn)-Gly-Ile-Gly-Cys(Trt)-Asn-Hyp(OTBS)-O- | 4a | 30% |
| | resin | | |
| 2 | Fmoc-Pro-Ile-Trp-Gly-Ile-Gly-Cys(Trt)-Asn(Trt)-O-resin | 4b | 42% |
| 3 | Fmoc-Hyp-Ile-Trp-Gly-Ile-Gly-Cys(Trt)-Asn(Trt)-O-resin | 4c | 50% |
| 4 | Fmoc-Ile-Trp-Gly-Ile-Gly-Cys(Trt)-Asn(Trt)-Hyp(OTBS)-O- | 4d | 45% |
| | resin | | |
| 5 | Fmoc-Trp-Gly-Ile-Gly-Cys(Trt)-Asn(Trt)-Hyp-Ile-O-resin | 4e | 25% |
| 6 | Fmoc-Asn(Trt)-Hyp-Ile-Trp-Gly-Ile-Gly-Cys(Trt)-O-resin | 4f | 27% |
| 7 | Fmoc-DhIle(OTBS)₂-Trp-Gly-Ile-Gly-Cys(Trt)-Asn- | 4g | 49% |
| | Hyp(OTBS)-O-resin | | |
| 8 | Fmoc-DhIle(OTBS)₂-Trp(6-OBn)-Gly-Ile-Gly-Cys(Trt)-Asn- | 4h | 38% |
| | Hyp(OTBS)-O-resin | | |
| 9 | Fmoc-Ile-Trp-Gly-Ile-Gly-Cys(Trt)-Asp(^{t}Bu)-Hyp(OTBS)-O- | 4i | 42% |
| | resin | | |
| 10 | Fmoc-DhIle(OTBS)₂-Trp-Gly-Ile-D-Pro-Cys(Trt)-Asn-Hyp(OTBS)- | 4j | 40% |
| | O-resin | | |
| 11 | Fmoc-DhIle(OTBS)₂-Trp-Gly-Ile-Gly-HomoCys(Trt)-Asn- | 4k | 40% |
| | Hyp(OTBS)-O-resin | | |
| 12 | Fmoc-Ile-Trp-Gly-Ile-D-Pro-Cys(Trt)-Asn-Hyp(OTBS)-O- | 4l | 41% |
| | resin | | |
| 13 | Fmoc-Thr^{t}Bu)-Trp-Gly-Ile-Gly-Cys(Trt)-Asn-Hyp(OTBS)- | 4m | 42% |
| | O-resin | | |
| 14 | Fmoc-Pen(Trt)-Trp-Gly-Ile-Gly-Cys(Trt)-Asn(Trt)- | 4n | 30% |
| | Hyp(OTBS)-O-resin | | |
| 15 | Fmoc-Ile-Trp-D-Pro-Ile-Gly-Cys(Trt)-Asn(Trt)-Hyp(OTBS)- | 4o | 43% |
| | O-resin | | |
| 16 | Fmoc-Ile-Trp-L-Pro-Ile-Gly-Cys(Trt)-Asn(Trt)-Hyp(OTBS)- | 4p | 45% |
| | O-resin | | |
| 17 | Fmoc-DhIle(OTBS)₂-Trp-D-Pro-Ile-Gly-Cys(Trt)-Asn(Trt)- | 4q | 38% |
| | Hyp(OTBS)-O-resin | | |
| 18 | Fmoc-DhIle(OTBS)₂-Trp-L-Pro-Ile-Gly-Cys(Trt)-Asn(Trt)- | 4r | 40% |
| | Hyp(OTBS)-O-resin | | |
| 19 | Fmoc-DhIle(OTBS)₂-Trp-Gly-Ile-Sar-Cys(Trt)-Asn-Hyp(OTBS)-O- | 4s | 40% |
| | resin | | |
| 20 | Fmoc-DhIle(OTBS)₂-Trp-Gly-Ile-Aib-Cys(Trt)-Asn-Hyp(OTBS)-O- | 4t | 38% |
| | resin | | |
| 21 | Fmoc-DhIle(OTBS)₂-Trp-Gly-Phe-Gly-Cys(Trt)-Asn-Hyp(OTBS)- | 4u | 35% |
| | O-resin | | |
| 22 | Fmoc-Trp-Gly-Thr(^{t}Bu)-Gly-Cys(Trt)-Asn-Hyp(OTBS)-O-resin | 4v | 20% |

Compound 4a: HPLC-MS: Retention time Rₜ=4.45 min (Gradient A); HRMS (ESI): m/z calculated: C₄₀H₅₂N₉O₁₁S⁺ [M+H]⁺ 866.3502. found 866.3494. Yield (purified after RP-HPLC): 77 mg.

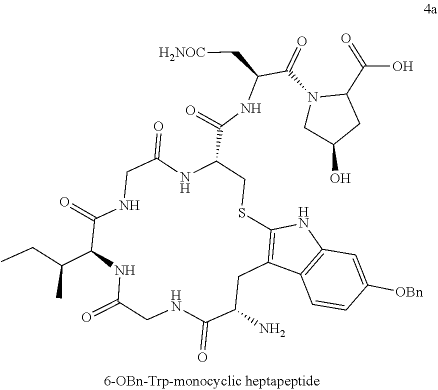

Compound 4b: HPLC-MS: Retention time Rₜ=7.11 min (Gradient A); HRMS (ESI): m/z calculated: C₅₈H₇₁N₁₀O₁₀S⁺ [M+H]⁺ 1099.5070. found 1099.5079. Yield (purified after RP-HPLC): 138 mg.

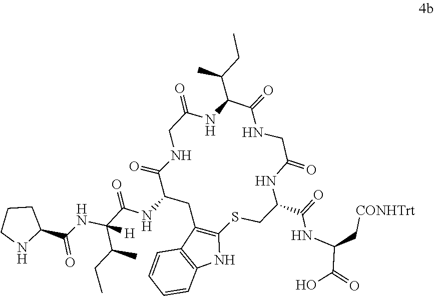

Compound 4c: HPLC-MS: Retention time Rₜ=6.98 min (Gradient A); HRMS (ESI): m/z calculated: C₅₈H₇₁N₁₀O₁₁S⁺ [M+H]⁺ 1115.5019., found 1115.5009. Yield (purified after RP-HPLC): 167 mg.

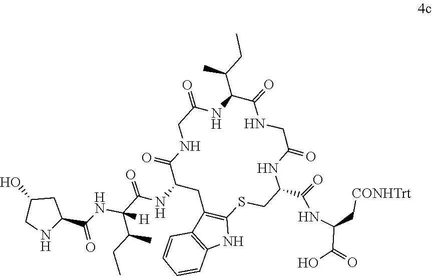

Compound 4d: HPLC-MS: Retention time Rₜ=7.72 min (Gradient B); HRMS (ESI): m/z calculated: C₃₉H₅₇N₁₀O₁₁S⁺ [M+H]⁺ 873.3923, found 873.3926. Yield (purified after RP-HPLC): 117 mg.

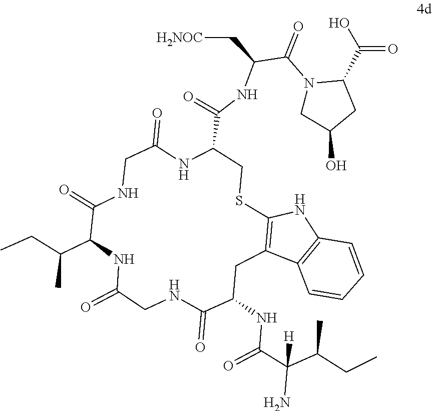 [SMILES: CC[C@H](C)[C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)CNC(=O)[C@H]([C@@H](C)CC)NC(=O)CNC1=O]

Compound 4e: HPLC-MS: Retention time Rₜ=8.19 min (Gradient B); HRMS (ESI): m/z calculated: C₃₉H₅₇N₁₀O₁₁S⁺ [M+H]⁺ 873.3923, found 873.3922. Yield (purified after RP-HPLC): 65 mg.

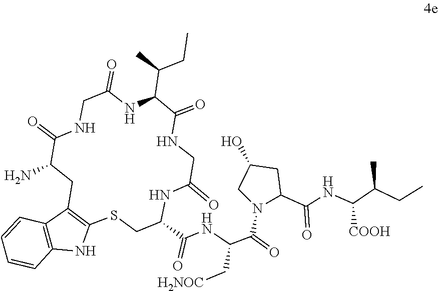

Compound 4f: HPLC-MS: Retention time Rₜ=8.33 min (Gradient B); HRMS (ESI): m/z calculated: C₃₉H₅₇N₁₀O₁₁S⁺ [M+H]⁺ 873.3923, found 873.3921. Yield (purified after RP-HPLC): 70 mg.

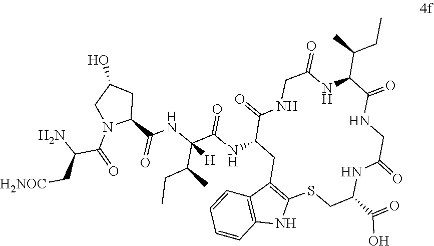 [SMILES: CC[C@H](C)[C@@H]1NC(=O)CNC(=O)[C@@H](NC(=O)[C@@H](NC(=O)[C@@H]2C[C@@H](O)CN2C(=O)[C@H](N)CC(N)=O)[C@@H](C)CC)Cc2c([nH]c3ccccc23)SC[C@@H](C(=O)O)NC(=O)CNC1=O]

Compound 4g: HPLC-MS: Retention time Rₜ=9.20 min (Gradient B); HRMS (ESI): m/z calculated: C₃₉H₅₇N₁₀O₁₃S⁺ [M+H]⁺ 905.3822, found 905.3833. Yield (purified after RP-HPLC): 133 mg.

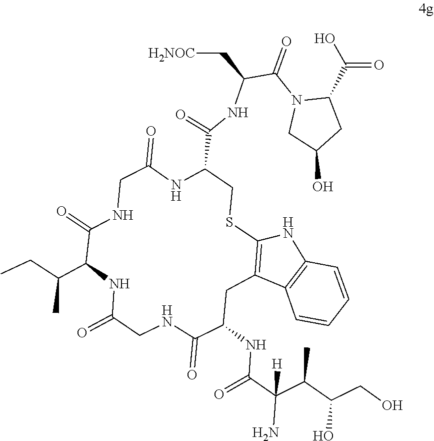 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

Compound 4h: HPLC-MS: Retention time Rₜ=9.43 min (Gradient B); HRMS (ESI): m/z calculated: C₄₆H₆₃N₁₀O₁₄S⁺ [M+H]⁺ 1011.4240, found 1011.4235. Yield (purified after RP-HPLC): 115 mg.

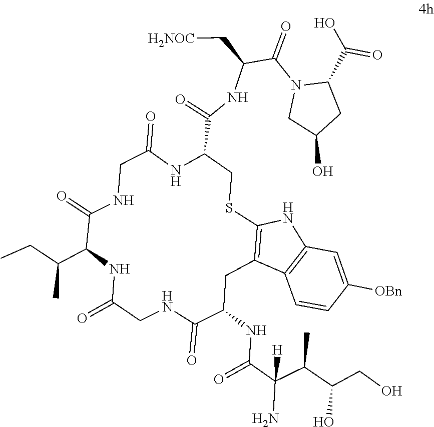 [SMILES: CC[C@H](C)[C@@H]1NC(=O)CNC(=O)[C@@H](NC(=O)[C@@H](N)[C@@H](C)[C@@H](O)CO)Cc2c([nH]c3cc(OCc4ccccc4)ccc23)SC[C@@H](C(=O)N[C@@H](CC(N)=O)C(=O)N2C[C@H](O)C[C@H]2C(=O)O)NC(=O)CNC1=O]

Compound 4i: HPLC-MS: Retention time Rₜ=9.56 min (Gradient A); HRMS (ESI): m/z calculated: C₄₃H₆₄N₉O₁₂S⁺ [M+H]⁺ 930.4390, found 930.4395 Yield (purified after RP-HPLC): 116 mg.

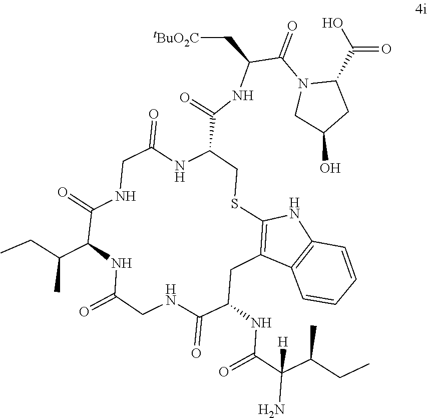 [SMILES: CC[C@H](C)[C@H](N)C(=O)N[C@H]1Cc2c([nH]c3ccccc23)SC[C@@H](C(=O)N[C@@H](CC(=O)OC(C)(C)C)C(=O)N2C[C@H](O)C[C@H]2C(=O)O)NC(=O)CNC(=O)[C@H]([C@@H](C)CC)NC(=O)CNC1=O]

Compound 4j: HPLC-MS: Retention time Rₜ=8.26 min (Gradient B); HRMS (ESI): m/z calculated: C₄₂H₆₁N₁₀O₁₃S⁺ [M+H]⁺ 945.4135, found 945.4133. Yield (purified after RP-HPLC): 113 mg.

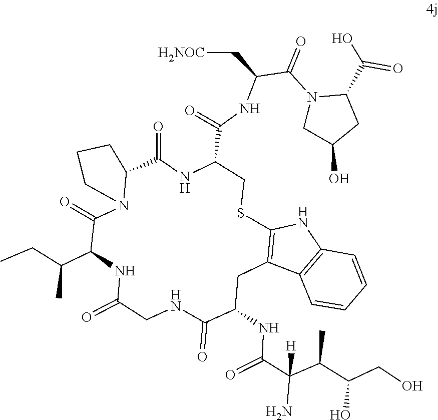 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)[C@H]2CCCN2C1=O]

Compound 4k: HPLC-MS: Retention time Rₜ=7.55 min (Gradient B); HRMS (ESI): m/z calculated: C₄₀H₅₉N₁₀O₁₃S⁺ [M+H]⁺ 919.3978, found 919.3987. Yield (purified after RP-HPLC): 110 mg.

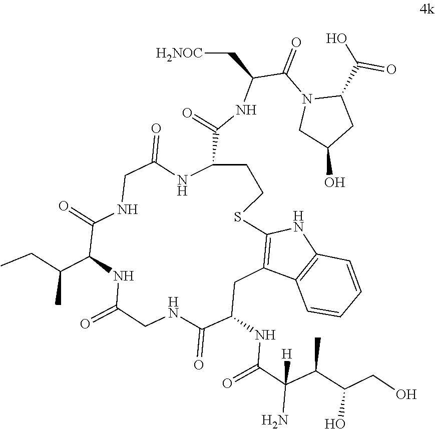 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SCC[C@@H](C(=O)N[C@@H](CC(N)=O)C(=O)N2C[C@H](O)C[C@H]2C(=O)O)NC(=O)CNC1=O]

Compound 4l: HPLC-MS: Retention time Rₜ=9.62 min (Gradient B); HRMS (ESI): m/z calculated: C₄₂H₆₁N₁₀O₁₁S⁺ [M+H]⁺ 913.4236, found 913.4240. Yield (purified after RP-HPLC): 112 mg.

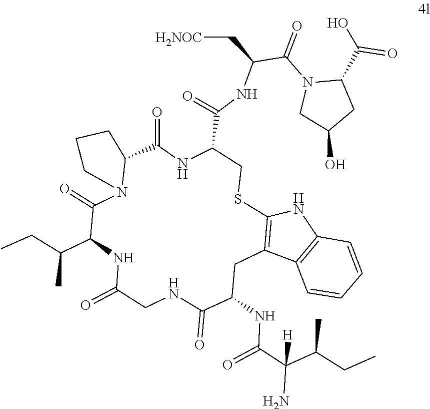 [SMILES: CC[C@H](C)[C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)[C@H]2CCCN2C(=O)[C@H]([C@@H](C)CC)NC(=O)CNC1=O]

Compound 4m: HPLC-MS: Retention time Rₜ=8.81 min (Gradient B); HRMS (ESI): m/z calculated: C₃₇H₅₃N₁₀O₁₂S [M+H]⁺, 861.3560 found 861.3563. Yield (purified after RP-HPLC): 108 mg.

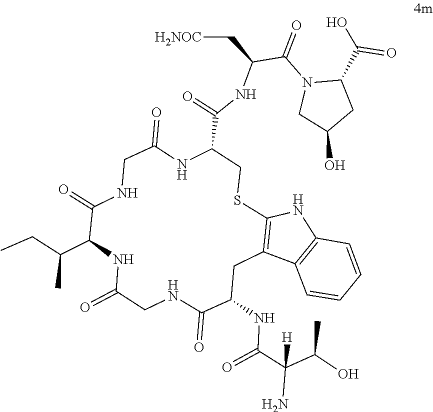 [SMILES: CC[C@H](C)[C@@H]1NC(=O)CNC(=O)[C@@H](NC(=O)[C@@H](N)[C@@H](C)O)Cc2c([nH]c3ccccc23)SC[C@@H](C(=O)N[C@@H](CC(N)=O)C(=O)N2C[C@H](O)C[C@H]2C(=O)O)NC(=O)CNC1=O]

Compound 4n: HPLC-MS: Retention time Rₜ=10.41 min (Gradient A); HRMS (ESI): m/z calculated: C₃₂H₉₇N₁₀O₁₁S₂Si⁺ [M+H]⁺ 1489.6543, found 1489.6553. Yield (purified after RP-HPLC): 134 mg.

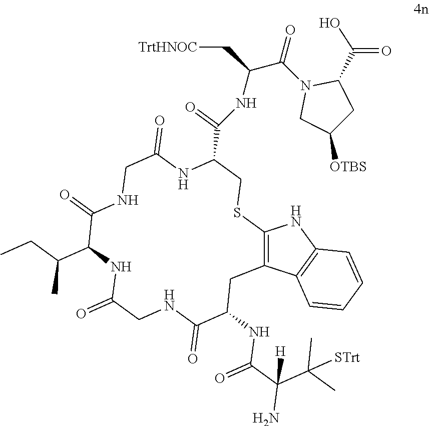

Compound 4o: HPLC-MS: Retention time Rₜ=9.72 min (Gradient B); HRMS (ESI): m/z calculated: C₄₂H₆₁N₁₀O₁₁S⁺ [M+H]⁺ 913.4236, found 913.4237. Yield (purified after RP-HPLC): 95 mg.

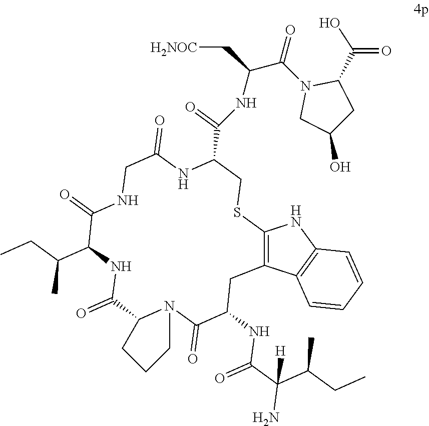 [SMILES: CC[C@H](C)[C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)CNC(=O)[C@H]([C@@H](C)CC)NC(=O)[C@H]2CCCN2C1=O]

Compound 4p: HPLC-MS: Retention time Rₜ=9.00 min (Gradient B); HRMS (ESI): m/z calculated: C₄₂H₆₁N₁₀O₁₁S⁺ [M+H]⁺ 913.4236, found 913.4232. Yield (purified after RP-HPLC): 101 mg.

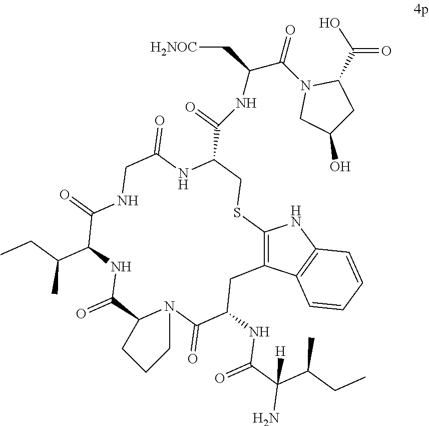 [SMILES: CC[C@H](C)[C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)CNC(=O)[C@H]([C@@H](C)CC)NC(=O)[C@@H]2CCCN2C1=O]

Compound 4q: HPLC-MS: Retention time Rₜ=6.19 min (Gradient B); HRMS (ESI): m/z calculated: C₄₂H₆₁N₁₀O₁₃S⁺ [M+H]⁺ 945.4135, found 945.4139. Yield (purified after RP-HPLC): 107 mg.

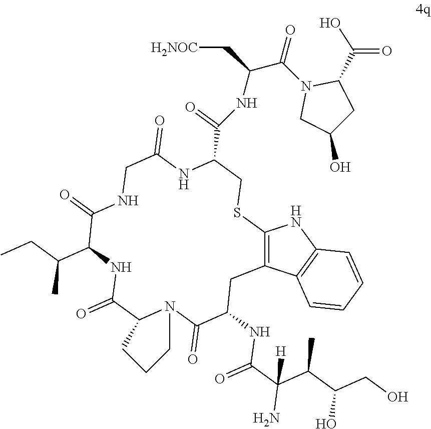 [SMILES: CC[C@H](C)[C@@H]1NC(=O)[C@H]2CCCN2C(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

Compound 4r: HPLC-MS: Retention time Rₜ=8.37 min (Gradient B); HRMS (ESI): m/z calculated: C₄₂H₆₁N₁₀O₁₃S⁺ [M+H]⁺ 945.4135, found 945.4134. Yield (purified after RP-HPLC): 113 mg.

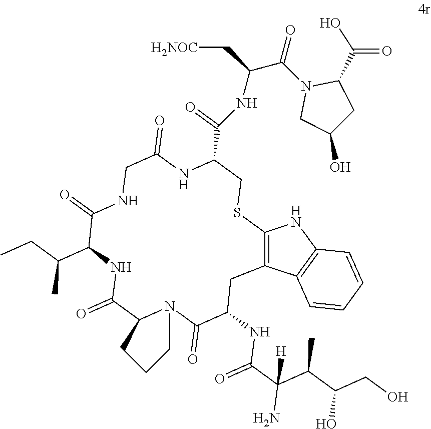 [SMILES: CC[C@H](C)[C@@H]1NC(=O)[C@@H]2CCCN2C(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

Compound 4s: HPLC-MS: Retention time Rₜ=6.20 min (Gradient B); HRMS (ESI): m/z calculated: C₄₀H₅₉N₁₀O₁₃S⁺ [M+H]⁺ 919.3978, found 919.3980. Yield (purified after RP-HPLC): 110 mg.

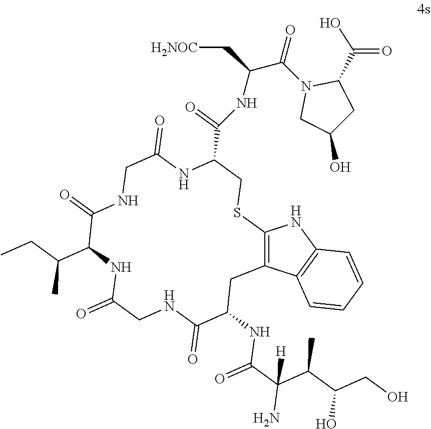 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

Compound 4t: HPLC-MS: Retention time Rₜ=7.95 min (Gradient B); HRMS (ESI): m/z calculated: C₄₁H₆₁N₁₀O₁₃S⁺ [M+H]⁺ 933.4135, found 933.4134. Yield (purified after RP-HPLC): 106 mg.

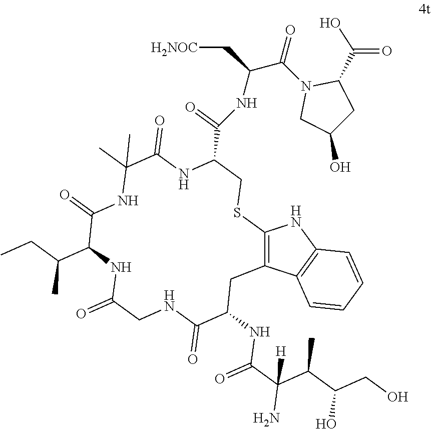 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)C(C)(C)NC1=O]

Compound 4u: HPLC-MS: Retention time Rₜ=7.40 min (Gradient B); HRMS (ESI): m/z calculated: C₄₂H₅₅N₁₀O₁₃S⁺ [M+H]⁺ 939.3665, found 939.3658. Yield (purified after RP-HPLC): 98 mg.

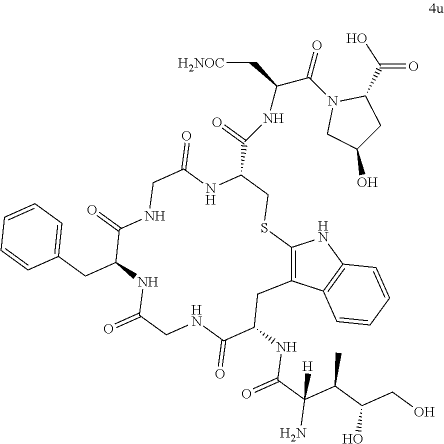 [SMILES: C[C@H]([C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)CNC(=O)[C@H](Cc2ccccc2)NC(=O)CNC1=O)[C@@H](O)CO]

Compound 4v: HPLC-MS: Retention time Rₜ=2.30 min (Gradient B); HRMS (ESI): m/z calculated: C₃₁H₄₂N₉O₁₁S⁺ [M+H]⁺ 748.2719, found 748.2714. Yield (purified after RP-HPLC): 44 mg.

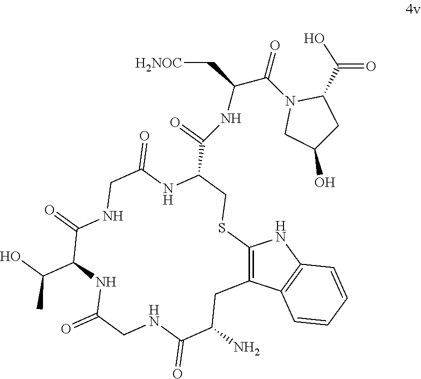 [SMILES: C[C@@H](O)[C@@H]1NC(=O)CNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)CNC1=O]

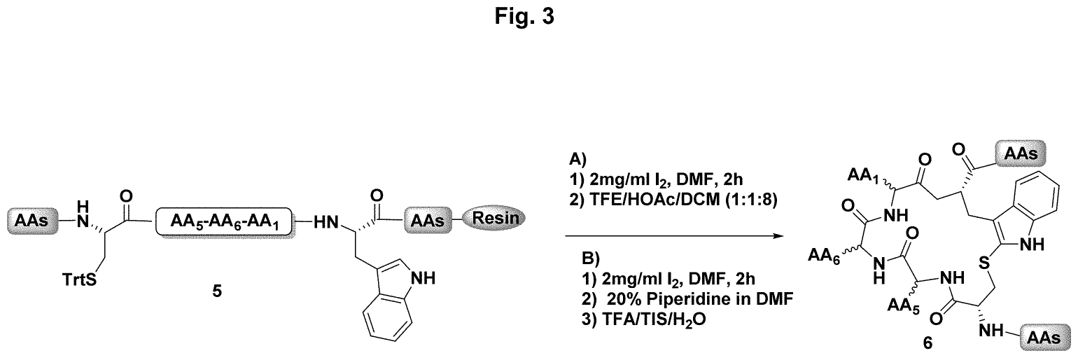

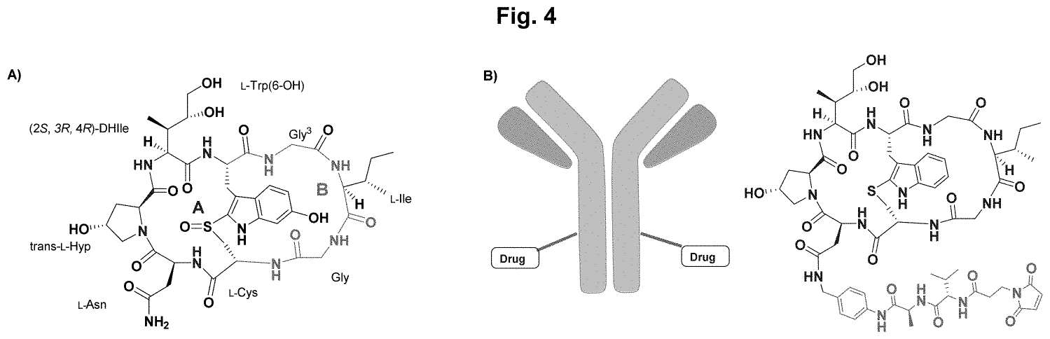

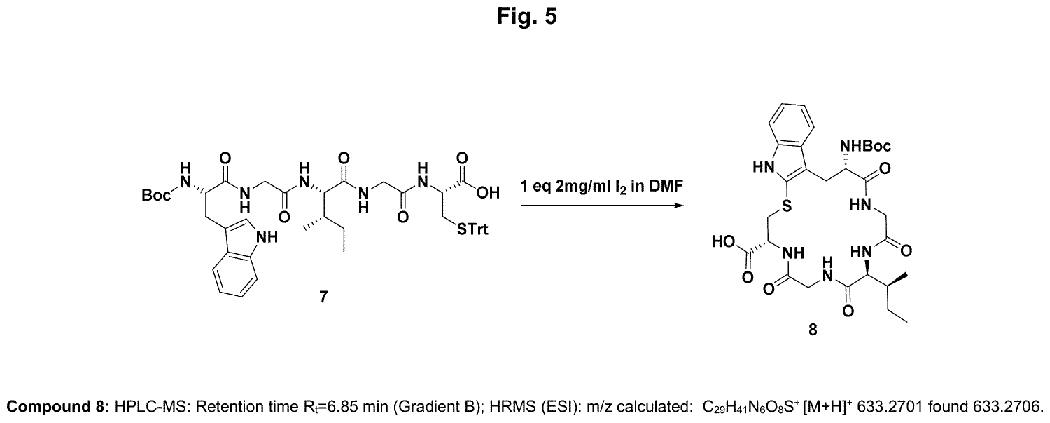

TABLE 3
Amino acid sequences of peptides bound to solid support cyclized by iodine.
The table shows examples where the cyclization took place via the A-ring
(N-terminal Cys and C-terminal Trp) (see FIG. 3).

| | Peptide bound on the resin | |
|---|---|---|
| Entry | (amino acid sequence) | Compound |
| 1 | Fmoc-Ile-Gly-Cys(Trt)-Asn(Trt)-Hyp(OTBS)-Ile-Trp-Gly- | 6a |
| | O-resin | |
| 2 | Fmoc-Ile-Gly-Cys(Trt)-Asn(Trt)-Ile-Hyp(OTBS)-Trp-Gly- | 6b |
| | O-resin | |
| 3 | Fmoc-Ile-Gly-Cys(Trt)-Ile-Hyp(OTBS)-Asn(Trt)-Trp-Gly- | 6c |
| | O-resin | |
| 4 | Fmoc-Ile-Cys(Trt)-Gly-Ile-Gly-Trp-Asn(Trt)-O-resin | 6d |
| 5 | Fmoc-Cys(Trt)-Asn(Trt)-trans-Hyp(OTBS)-Ile-Trp-Gly- | 6e |
| | Ile-Gly-O-resin | |
| 6 | Fmoc-Gly-Cys(Trt)-Asn(Trt)-trans-Hyp(OTBS)-Ile-Trp- | 6f |
| | Gly-Ile-O-resin | |
| 7 | Fmoc-Gly-Ile-Gly-Cys(Trt)-Asn(Trt)-trans-Hyp(OTBS)- | 6g |
| | Ile-Trp-O-resin | |
| 8 | Fmoc-Ile-Gly-Cys(Trt)-Asn(Trt)-cis-Hyp(OTBS)-Ile-Trp- | 6h |
| | Gly-O-resin | |

Analytical Data:

Compound 6a: HPLC-MS: Retention time Rₜ=10.92 min (Gradient C); HRMS (ESI): m/z calculated: C₇₉H₉₅N₁₀O₁₃SSi⁺ [M+H]⁺ 1451.6565 found 1451.6564.

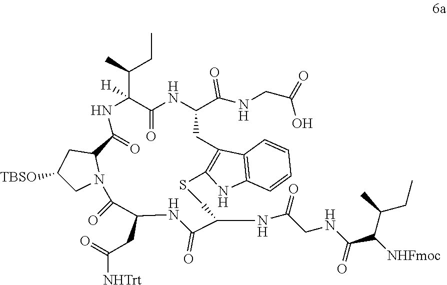

Compound 6b: HPLC-MS: Retention time Rₜ=10.93 min (Gradient C); HRMS (ESI): m/z calculated: C₇₉H₉₅N₁₀O₁₃SSi⁺ [M+H]⁺ 1451.6565 found 1451.6548.

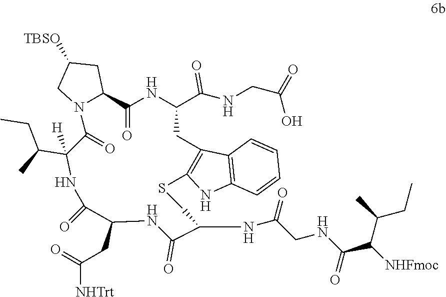

Compound 6c: HPLC-MS: Retention time Rₜ=11.21 min (Gradient C); HRMS (ESI): m/z calculated: C₇₉H₉₅N₁₀O₁₃SSi⁺ [M+H]⁺ 1451.6565 found 1451.6559.

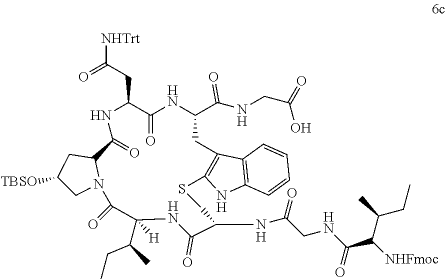

Compound 6d: HPLC-MS: Retention time Rₜ=7.70 min (Gradient C); HRMS (ESI): m/z calculated: C₆₈H₇₄N₉O₁₁S [M+H]⁺ 1224.5223 found 1224.5222.

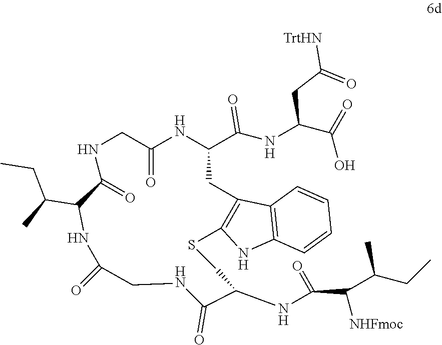

Compound 6e: HPLC-MS: Retention time Rₜ=8.72 min (Gradient B); HRMS (ESI): m/z calculated: C₃₉H₅₇N₁₀O₁₁S⁺ [M+H]⁺ 873.3923, found 873.3910.

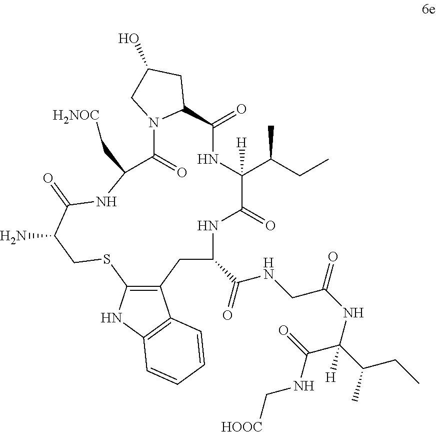

Compound 6f: HPLC-MS: Retention time Rₜ=7.68 min (Gradient B); HRMS (ESI): m/z calculated: C₃₉H₅₇N₁₀O₁₁S⁺ [M+H]⁺ 873.3923, found 873.3914.

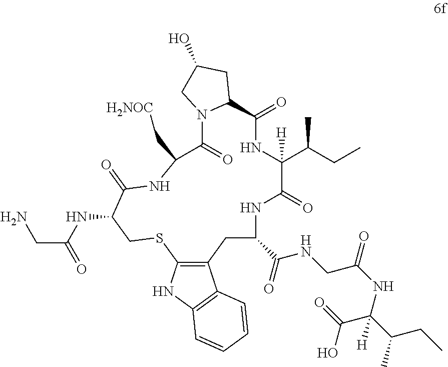

Compound 6g: HPLC-MS: Retention time Rₜ=8.08 min (Gradient B); HRMS (ESI): m/z calculated: C₃₉H₅₇N₁₀O₁₁S⁺ [M+H]⁺ 873.3923, found 873.3909.

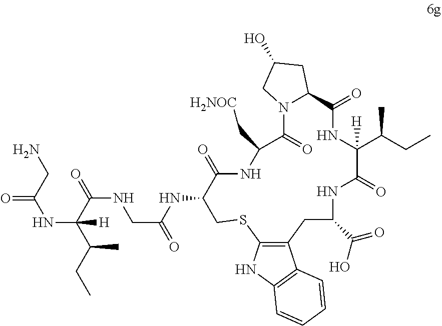 [SMILES: CC[C@H](C)[C@H](NC(=O)CN)C(=O)NCC(=O)N[C@H]1CSc2[nH]c3ccccc3c2C[C@@H](C(=O)O)NC(=O)[C@H]([C@@H](C)CC)NC(=O)[C@@H]2C[C@@H](O)CN2C(=O)[C@H](CC(N)=O)NC1=O]

Compound 6h: HPLC-MS: Retention time Rₜ=11.23 min (Gradient C); HRMS (ESI): m/z calculated: C₇₉H₉₅N₁₀O₁₃SSi⁺ [M+H]⁺ 1451.6565 found 1451.6526.

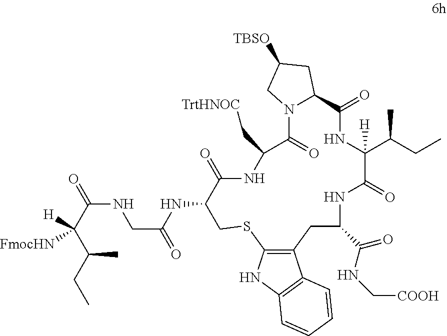

TABLE 4
Amino acid sequences of peptides bound to solid support not cyclized by iodine.

| Entry | Peptide bound to the resin (amino acid sequence) | Short description |
|---|---|---|
| 1 | Fmoc-Ile-Gly-Cys(Trt)-Asn(Trt)-Hyp(OH)-Ile-Trp- | No protected hydroxyl group of |
| | Gly-O-resin | Hyp |
| 2 | Fmoc-Gly-Cys(Trt)-Asn(Trt)-Hyp(OH)-Ile-Trp-Gly- | No protected hydroxyl group of |
| | Ile-O-resin | Hyp |
| 3 | Fmoc-Cys(Trt)-Asn(Trt)-Hyp(OH)-Ile-Trp-Gly-Ile- | No protected hydroxyl group of |
| | Gly-O-resin | Hyp |
| 4 | Fmoc-Gly-Ile-Gly-Cys(Trt)-Asn(Trt)-Hyp(OH)-Ile- | No protected hydroxyl group of |
| | Trp-O-resin | Hyp |
| 5 | Fmoc-Ile-Gly-Cys(Trt)-Asn(Trt)-Pro-Ile-Trp-Gly-OH | Cyclization product in traces |
| 6 | Fmoc-Trp-Gly-Cys(Trt)-Gly-Cys(Trt)-O-resin | Disulfide bond formed between |
| | | two Cys |
| 7 | Fmoc-Trp-Gly-Ile-L-Pro-Cys(Trt)-O-resin | Stereochemistry of Pro |
| 8 | Fmoc-Trp(Boc)-Gly-Ile-Gly-Cys(Trt)-O-resin | Boc protected side chain of Trp |
| 9 | Fmoc-Trp-Gly-Ile-Gly-Pen(Trt)-O-resin | Cys replaced with Penicillamine |
| 10 | Fmoc-Gly-Trp-Cys(Trt)-Gly-O-resin | Much smaller size of the ring |
| 11 | Fmoc-5-OHTrp-Gly-Ile-Gly-Cys(Trt)-O-resin | No protected hydroxyl group of |
| | | Trp |
| 12 | 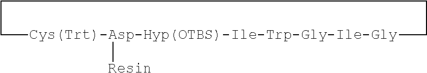 | Construction thioether of Macrocyclic lactam |

For compounds comprised in Table 4, the yields of cyclized peptides were below 5%.

TABLE 5
Synthesized bicyclic peptides from monocyclic precursors.

| | Peptides | |
|---|---|---|
| Entry | (linear amino acid sequence) | amatoxins |
| 1 | H₂N-Thr-Trp-Gly-Ile-Gly-Cys-Asn-Hyp-OH | 7a |
| 2 | H₂N-Ile-Trp-Gly-Ile-Gly-Cys-Asn-Hyp-OH | 7b |
| 3 | H₂N-Ile-Trp-Gly-Ile-Gly-Cys-Asp-Hyp-OH | 7c |
| 4 | H₂N-DhIle-Trp-Gly-Ile-Gly-Cys-Asn-Hyp-OH | 7d |
| 5 | H₂N-DhIle-Trp(6-OH)-Gly-Ile-Gly-Cys-Asn-Hyp-OH | 7e |
| 6 | Alpha-amanitin | 7f |
| 7 | H₂N-Ile-Trp-Gly-Ile-Gly-Cys-Asn-D-Pro-OH | 7g |
| 8 | H₂N-Ile-Trp-Gly-Ile-D-Pro-Cys-Asn-Hyp-OH | 7h |
| 9 | H₂N-DhIle-Trp-Gly-Ile-D-Pro-Cys-Asn-Hyp-OH | 7i |
| 10 | H₂N-DhIle-Trp-Gly-Ile-Gly-HomoCys-Asn-Hyp-OH | 7j |
| 11 | H₂N-DhIle-Trp(6-OBn)-Gly-Ile-Gly-Cys-Asn-Hyp-OH | 7k |
| 12 | H₂N-DhIle-Trp-Gly-Ile-Sar-Cys-Asn-Hyp-OH ^{(#)} | 7l |
| 13 | H₂N-DhIle-Trp-Gly-Ile-Aib-Cys-Asn-Hyp-OH ^{(§)} | 7m |
| 14 | H₂N-DhIle-Trp-Gly-Ile-Gly-Cys-Asn-Aze-OH^{(*)} | 7n |
| 15 | H₂N-DhIle-Trp-Gly-Ile-D-Ala-Cys-Asn-Hyp-OH | 7o |
| 16 | H₂N-DhIle-Trp-Gly-Tle-Gly-Cys-Asn-Hyp-OH^{(**)} | 7p |
| 17 | H₂N-DhIle-Trp-Gly-Ile-Gly-Cys-Asn-4-(F₂)Pro-OH | 7q |
| 18 | H₂N-DhIle-Trp-Gly-Ile-Acc-Cys-Asn-Hyp-OH^{(***)} | 7r |

^{#}: Sar: sarcosine;
^{§}: Aib: α-aminoisobutyric acid;
^{(*)}aze: azetidine;
^{(**)}Tle: -tert-leucine;
^{(***)}Acc: aminocyclopropane-1-carboxylic acid

Analytical Data:

Compound 7a: HPLC-MS: Retention time Rₜ=8.34 min (Gradient B); HRMS (ESI): m/z calculated: C₃₇H₅₁N₁₀O₁₁S⁺ [M+H]⁺ 843.3454 found 843.3461.

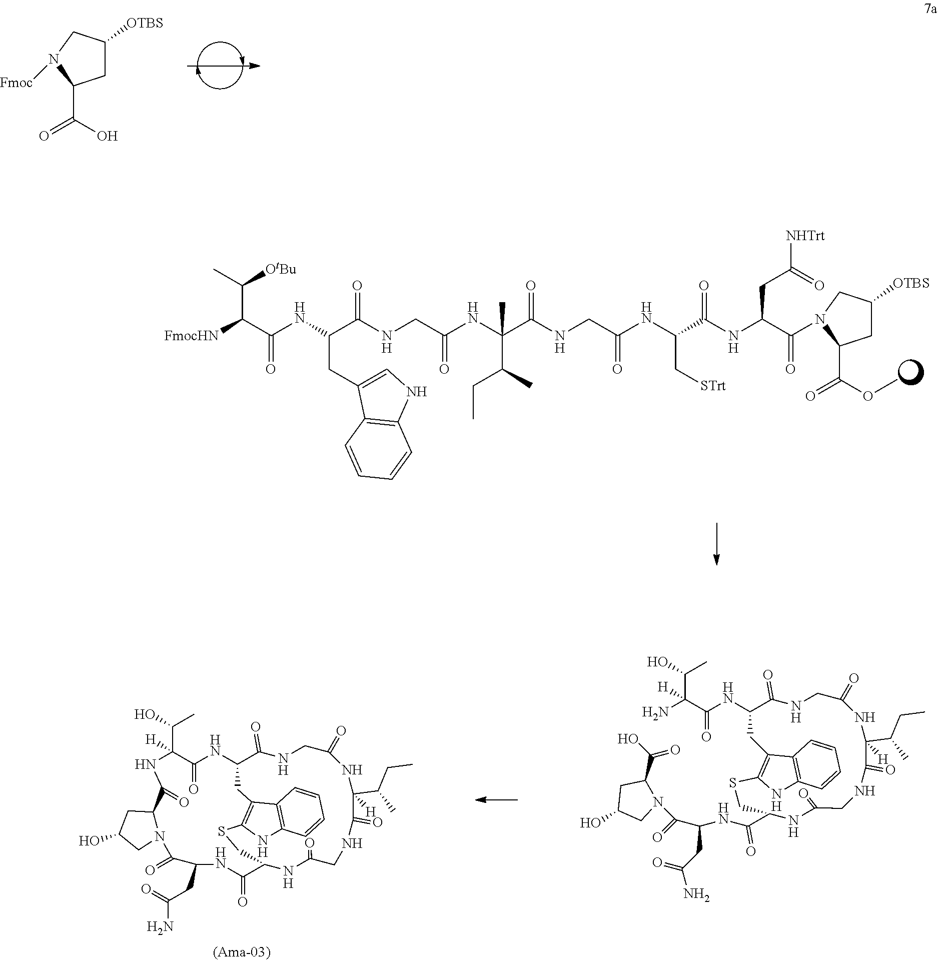

Synthesis of Compound 7a:

2-CTC resin (1 g, 0.98 mmol/g) was loaded with Fmoc-L-trans-Hyp(OTBS)—OH as described (cf. Material and Methods). The resin loading was determined to be 0.30 mmol/g. The Fmoc group was removed according to Method A. The following peptide sequences Fmoc-L-Asn(Trt)-OH (4 eq), Fmoc-L-Cys(Trt)-OH (4 eq), Fmoc-Gly-OH (4 eq), Fmoc-L-Ile-OH (4 eq), Fmoc-Gly-OH (4 eq), Fmoc-L-Trp-OH (4 eq) and Fmoc-L-(2S,3R,4R)-(TBS)₂-DHIle-OH (2 eq) was coupled to the deprotected resin according to Method A and B. The tryptathionine formation was carried according to the procedure disclosed herein (cf. Material and Methods). After removal of Fmoc group and followed cleavage from the resin using TFA/TIS/H₂O (95:2.5:2.5), the crude peptides was used for next step without further purification. Obtained monocyclic octapeptide (86 mg, 100 μmol, 1.0 eq) was dissolved in DMF (50 mL). Then, DIPEA (2.2 eq) and HATU (2.0 eq) was added at 0° C. The reaction mixture was allowed to warm to r.t. for 12 h and concentrated under reduced pressure. The crude product was purified using preparative HPLC to afford bicyclic octapeptide compound 7a (32 mg, yield: 38%) as a white powder.

Compound 7b: HPLC-MS: Retention time Rₜ=5.73 min (Gradient A); HRMS (ESI): m/z calculated: C₃₉H₅₅N₁₀O₁₀S⁺ [M+H]⁺ 855.3878 found 855.3839.

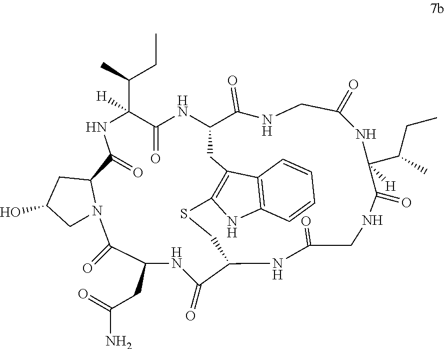 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

Synthesis of compound 7b was done in analogous fashion to compound 7a as disclosed above using the corresponding Fmoc-protected amino acids. The crude product was purified using preparative HPLC to afford bicyclic octapeptide 7b (20 mg, 68%) as a white powder.

Compound 7c: HPLC-MS: Retention time Rₜ=6.75 min (Gradient B); HRMS (ESI): m/z calculated: C₃₉H₅₄N₉O₁₁S⁺ [M+H]⁺ 856.3658 found 856.3660.

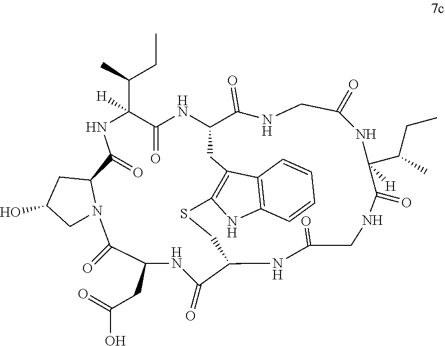 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(=O)O)C(=O)N1C[C@H](O)C[C@H]1C(=O)N[C@@H]([C@@H](C)CC)C(=O)N2]

Synthesis of compound 7c was done analogously to the synthesis of compound 7a (Ama-03) using the corresponding Fmoc-protected amino acids. The crude product was purified using preparative HPLC to afford bicyclic octapeptide 7c (30 mg, 60%) as a white powder.

Compound 7d: HPLC-MS: Retention time Rₜ=6.76 min (Gradient B); HRMS (ESI): m/z calculated: C₃₉H₅₅N₁₀O₁₂S⁺ [M+H]⁺ 887.3716 found 887.3727.

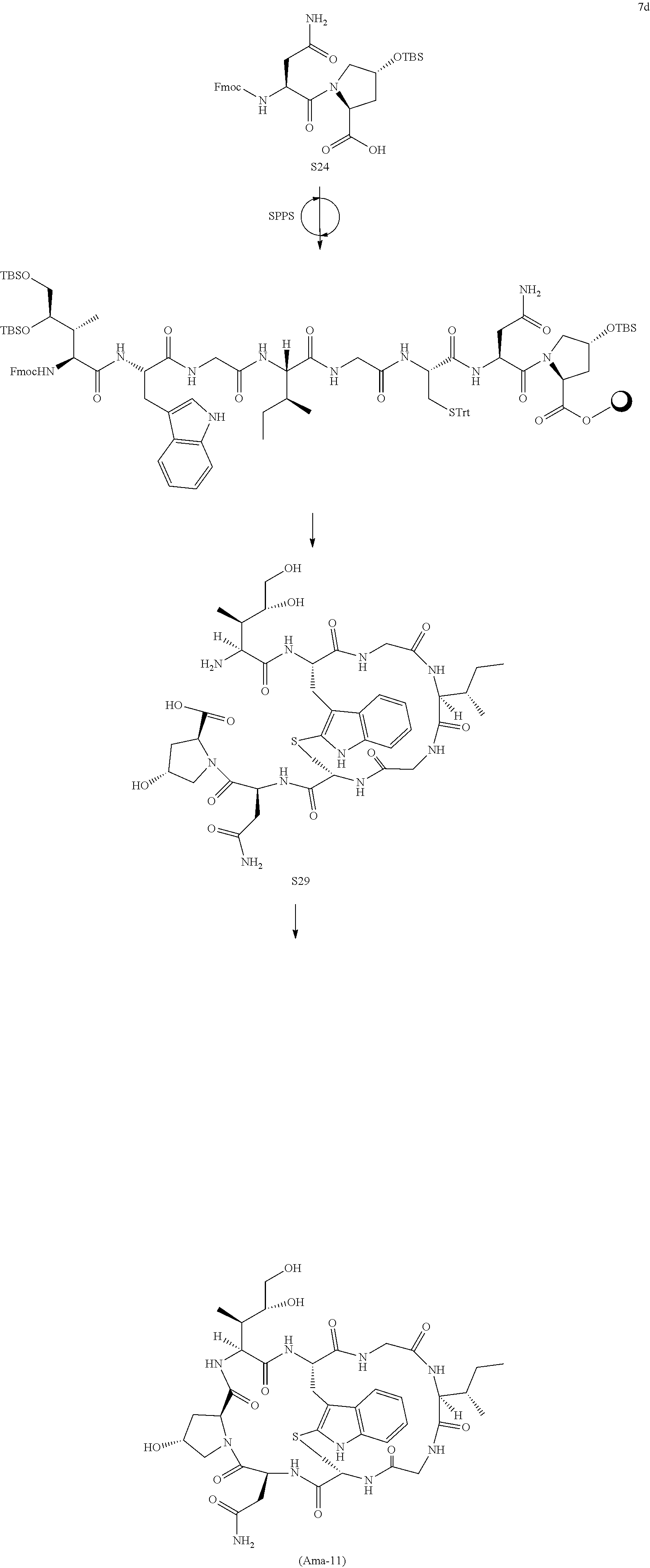

Synthesis of Compound 7d:

2-CTC resin (1 g, 0.98 mmol/g) was loaded with S₂₄ as described (cf. Material and Methods). The resin loading was determined to be 0.30 mmol/g. The Fmoc group was removed according to Method A. Fmoc-Cys(Trt)-OH (4 eq) was coupled to the deprotected resin according to Method B. The Fmoc group of the resulting resin was removed according to Method A. The following peptide sequences Fmoc-Gly-OH (4 eq), Fmoc-L-Ile-OH (4 eq), Fmoc-Gly-OH (4 eq), Fmoc-L-Trp-OH (4 eq) and Fmoc-L-(2S,3R,4R)-(TBS)₂-DHIle-OH (2 eq) was coupled to the deprotected resin according to Method A and B. The tryptathionine formation was carried out on the solid support (cf. Material and Methods). After removal of Fmoc group using method A and followed cleavage from the resin using condition B, 125 mg crude monocyclic peptide S₂₉ was obtained after desilylation with TBAF in THF (10 eq) (cf. Material and Methods). Subsequent HPLC purification afforded S₂₉ in a 46% overall yields.

Compound 7e: HPLC-MS: Retention time Rₜ=6.70 min (Gradient B); HRMS (ESI): m/z calculated: C₃₉H₅₅N₁₀O₁₃S⁺ [M+H]⁺ 903.3665 found 903.3662.

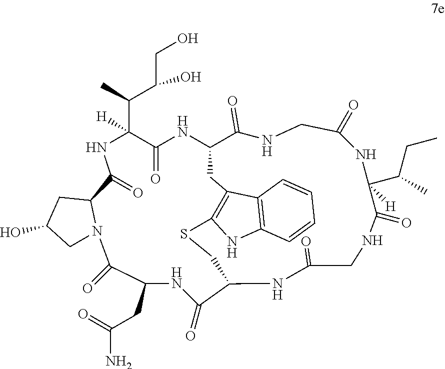 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

Synthesis of compound 7e is part of the total synthesis of 7f (alpha-amanitin) and disclosed herein.

Compound 7f: HPLC-MS: Retention time Rₜ=6.36 min (Gradient B); HRMS (ESI): m/z calculated: C₃₉H₅₅N₁₀O₁₄S⁺ [M+H]⁺ 919.3614 found 919.3632.

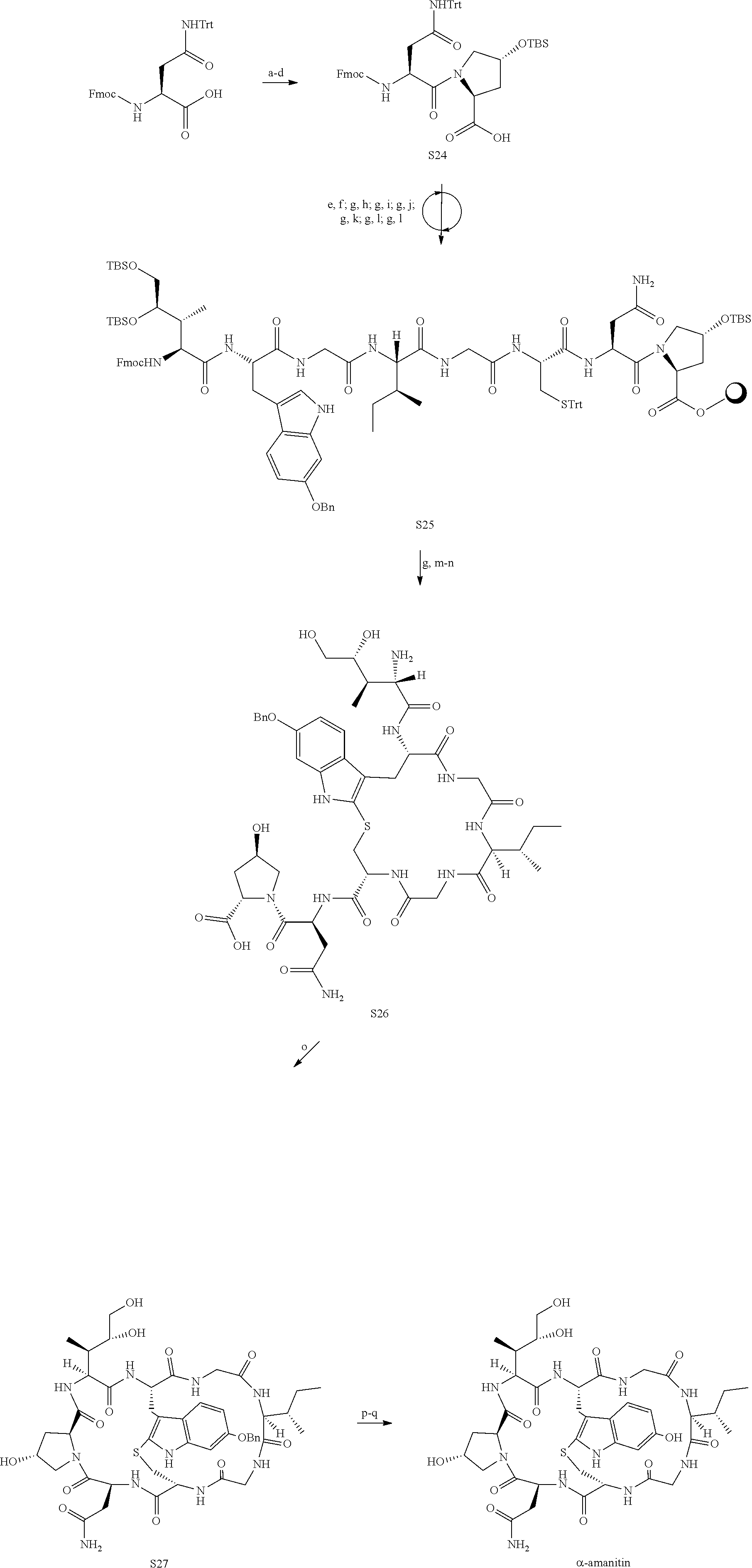

Total Synthesis of α-Amanitin

a) DCC, NHS, DCM; b) H-Hyp-OH, NaHCO₃, dioxane/H₂O; c) TFA/DCM (1:1), 1 h; d) TBDMSOTf, lutidine, DCM, 16 h; e) 2-CTC-resin, DCM, DIPEA; f) MeOH/DIPEA/DCM (1:1:8); g) piperdine/DMF (1:4); h) Fmoc-L-Cys-OH, TBTU, DIPEA, DMF; h) Fmoc-Gly-OH, TBTU, DIPEA, DMF; i) Fmoc-Ile-OH, TBTU, DIPEA, DMF; j) Fmoc-Gly-OH, TBTU, DIPEA, DMF; k) Fmoc-L-6-OBn-Trp-OH (15), TBTU, DIPEA, DMF; I) Fmoc-L-(2S,3R,4R)-(TBS)₂-DHIle-OH (16), TBTU, DIPEA, DMF; m) TFE/HOAc/DCM (1:1:8); n) TBAF, THF; o) HATU, DIPEA, DMF/DCM; p) BF₃-Et₂O, EtSH q) mCPBA.

2-CTC resin (1 g, 0.98 mmol/g) was loaded with S₂₄ as described (cf. Material and Methods). The resin loading was determined to be 0.30 mmol/g. The Fmoc group was removed according to Method A. Fmoc-Cys(Trt)-OH (4 eq) was coupled to the deprotected resin according to Method B. The Fmoc group of the resulting resin was removed according to Method A. The following peptide sequences Fmoc-Gly-OH (4 eq), Fmoc-L-Ile-OH (4 eq), Fmoc-Gly-OH (4 eq), Fmoc-L-6-OBn-Trp-OH (4 eq) and Fmoc-L-(2S, 3R, 4R)-(TBS)₂-DHIle-OH (2 eq) was coupled to the deprotected resin according to Method A and B. The tryptathionine formation was carried out on the solid support using method C. After removal of Fmoc group using method A and followed cleavage from the resin using condition B, 120 mg S₂₆ was obtained after desilylation with TBAF in THF (10 eq) (cf. Material and Methods). Subsequent HPLC purification afforded S₂₆ in a 40% overall yields.

HRMS (ESI): m/z calc. for C₄₆H₆₂N₁₀O₁₄S (M+H)⁺ 1011.4240, found 1011.4242.

HPLC-MS: Retention time Rₜ=8.78 min (Gradient C).

Monocyclic octapeptide S₂₆ (80 mg, 79 μmol, 1.0 eq) was dissolved in DMF (40 mL). Then, DIPEA (30 μL, 2.2 eq) and HATU (20 mg, 2.0 eq) was added at 0° C. The reaction mixture was allowed to warm to r.t. for 12 h and concentrated under reduced pressure. The crude product was purified using preparative HPLC to afford bicyclic octapeptide S27 (52 mg, 68%) as a white powder.

¹H NMR (500 MHz, DMSO-d₆) δ=11.03 (s, 1H), 8.83 (s, 1H), 8.51 (d, J=3.8 Hz, 1H), 8.42 (d, J=3.2 Hz, 1H), 8.13 (s, 1H), 8.08 (d, J=8.2 Hz, 1H), 7.99 (d, J=10.0 Hz, 1H), 7.95 (d, J=9.6 Hz, 1H), 7.87 (d, J=7.9 Hz, 1H), 7.49-7.38 (m, 8H), 7.33 (t, J=7.3 Hz, 1H), 6.81 (d, J=1.9 Hz, 1H), 6.77 (dd, J=8.8, 2.2 Hz, 1H), 5.25 (s, 1H), 5.10 (s, 2H), 4.98-4.90 (m, 1H), 4.83 (s, 1H), 4.71 (dd, J=6.9, 3.3 Hz, 1H), 4.55 (dd, J=17.3, 8.3 Hz, 1H), 4.45 (dd, J=9.3, 5.7 Hz, 1H), 4.40 (s, 1H), 4.28 (dd, J=11.4, 7.0 Hz, 1H), 4.18 (dd, J=18.8, 8.4 Hz, 1H), 3.91 (dd, J=17.3, 7.7 Hz, 1H), 3.82-3.68 (m, 2H), 3.08-2.90 (m, 3H), 2.76 (d, J=7.7 Hz, 1H), 2.25-2.15 (m, 2H), 1.88 (t, J=10.7 Hz, 1H), 1.60-1.55 (m, 1H), 1.26-1.22 (m, 1H), 1.17 (t, J=7.2 Hz, 3H), 1.14-1.07 (m, 1H), 0.89 (d, J=7.0 Hz, 3H), 0.88-0.84 (m, 1H), 0.83 (t, J=7.2 Hz, 3H), 0.79 (d, J=6.6 Hz, 3H).

HRMS (ESI): m/z calculated: C₄₆H₆₁N₁₀O₁₃S⁺ [M+H]⁺ 993.4135, found 993.4134.

HPLC-MS: Retention time Rₜ=10.24 min (Gradient B).

Bicyclic octapeptide S27 (16 mg, 16 μmol, 1.0 eq) was dissolved in EtSH (2 mL) and treated with BF₃—OEt₂ (45 μL, 20.0 eq) under vigorous stirring for 2 h after which the fully deprotected bicyclic octapeptide was precipitated in Et₂O. 11 mg of pure 6-OH—S-deoxo-amanitin (compound 7e) was obtained as white solid powder after HPLC purification in 80% yield.

¹H NMR (700 MHz, DMSO-d₆) δ=10.80 (s, 1H), 8.82 (s, 1H), 8.52 (s, 1H), 8.44 (d, J=3.0 Hz, 1H), 8.20 (s, 1H), 8.13 (s, 1H), 8.08 (d, J=8.2 Hz, 1H), 8.02 (d, J=10.1 Hz, 1H), 7.99-7.94 (m, 2H), 7.86 (d, J=8.0 Hz, 1H), 7.35 (dd, J=16.9, 8.6 Hz, 1H), 7.48 (s, 1H), 7.44 (t, J=3.5 Hz, 1H), 6.60 (d, J=1.4 Hz, 1H), 6.55 (dt, J=8.6, 2.0 Hz, 1H), 4.94-4.85 (m, 1H), 4.72-4.68 (m, 1H), 4.60-4.50 (m, 1H), 4.48 (dd, J=13.8, 7.1 Hz, 1H), 4.45 (dd, J=9.4, 5.8 Hz, 1H), 4.43-4.38 (m, 2H), 4.28 (dd, J=11.3, 7.2 Hz, 1H), 4.22-4.15 (m, 1H), 4.01-3.98 (m, 1H), 3.91 (dd, J=17.2, 7.4 Hz, 1H), 3.80-3.70 (m, 3H), 3.39-3.25 (m, 3H), 3.22-3.17 (m, 1H), 3.04-2.91 (m, 2H), 2.76-2.72 (m, 1H), 2.20-2.16 (m, 2H), 1.95-1.86 (m, 1H), 1.61-1.52 (m, 2H), 1.14-1.07 (m, 1H), 0.91-0.86 (m, 1H), 0.88 (d, J=6.9 Hz, 3H), 0.83 (t, J=7.2 Hz, 3H), 0.79 (d, J=6.2 Hz, 3H).

¹³C NMR (HSQC, 176 MHz, DMSO-d₆) δ=130.7, 129.9, 121.3, 110.1, 96.3, 75.9, 72.8, 69.1, 66.4, 64.0, 62.3, 59.5, 56.3, 56.2, 55.5, 55.1, 52.9, 53.9, 51.0, 44.1, 42.8, 42.6, 42.3, 42.1, 41.8, 39.6, 39.0, 38.1, 34.9, 34.5, 30.8, 29.3, 25.7, 15.3, 14.1, 11.0 ppm.

HRMS (ESI): m/z calculated: C₃₉H₅₅N₁₀O₁₃S⁺ [M+H]⁺ 903.3665, found 903.3669.

HPLC-MS: Retention time Rₜ=6.66 min (Gradient B).

The obtained white powder 6-OH—S-deoxo-amanitin (compound 7e) (8 mg, 8.8 μmol, 1.0 eq) was dissolved in 2 mL of a mixture of i-PrOH/EtOH (2:1). Then, a solution of mCPBA (0.7 eq) in i-PrOH/EtOH (2:1) was added dropwise. The resulting solution of was stirred for 30 min after which the reaction was terminated by the addition of water (1 mL). The crude product was purified via preparative HPLC which afforded α-amanitin (3 mg, 38%, compound 7f) as a white powder.

¹H NMR (500 MHz, DMSO-d₆) δ=11.25 (s, 1H), 9.20 (s, 1H), 8.74 (t, J=5.0 Hz, 1H), 8.50 (d, J=3.0 Hz, 1H), 8.44 (s, 1H), 8.39 (s, 1H), 8.30 (d, J=10.5 Hz, 1H), 7.97 (d, J=9.2 Hz, 1H), 7.89 (d, J=7.6 Hz, 1H), 7.80 (d, J=9.8 Hz, 1H), 7.58 (s, 1H), 7.44 (d, J=8.8 Hz, 1H), 6.75 (d, J=2.2 Hz, 1H), 6.60 (dd, J=8.7, 2.1 Hz, 1H), 5.17 (d, J=3.0 Hz, 1H), 4.98-4.88 (m, 2H), 4.70 (d, J=5.1 Hz, 1H), 4.65 (dd, J=6.3, 3.5 Hz, 1H), 4.41 (dd, J=9.7, 7.2 Hz, 1H), 4.39-4.35 (m, 2H), 4.30 (dd, J=16.9, 7.5 Hz, 1H), 4.26 (dd, J=11.7, 6.6 Hz, 1H), 3.91 (dd, J=17.6, 7.2 Hz, 1H), 3.80 (d, J=8.7 Hz, 1H), 3.73 (d, J=11.3 Hz, 1H), 3.67 (d, J=6.5 Hz, 1H), 3.52-3.43 (m, 2H), 3.21 (dd, J=15.1, 7.3 Hz, 1H), 3.08 (t, J=13.3 Hz, 1H), 2.98-2.92 (m, 2H), 2.78-2.69 (m, 1H), 2.19 (dd, J=12.7, 6.6 Hz, 1H), 2.10 (dd, J=13.8, 6.8 Hz, 1H), 1.85 (t, J=10.6 Hz, 1H), 1.58-1.53 (m, 1H), 0.87 (d, J=7.0 Hz, 3H), 0.86-0.82 (m, 1H), 0.83 (t, J=7.3 Hz, 3H), 0.80 (d, J=6.8 Hz, 3H).

¹³C NMR (HSQC, 126 MHz, DMSO-d₆) δ=122.7, 111.2, 97.0, 72.6, 69.1, 63.9, 62.2, 59.6, 59.4, 56.1, 55.7, 53.4, 51.2, 50.7, 42.8, 38.5, 35.1, 29.2, 25.7, 15.3, 13.9, 11.3, ppm.

HRMS (ESI): m/z calculated: C₃₉H₅₅N₁₀O₁₄S⁺ [M+H]⁺ 919.3614, found 919.3639.

HPLC-MS: Retention time Rₜ=5.93 min (Gradient B).

Compound 7g: HPLC-MS: Retention time Rₜ=6.24 min (Gradient A); HRMS (ESI): m/z calculated: C₃₉H₅₅N₁₀O₉S⁺ [M+H]⁺ 839.3869 found 839.3902.

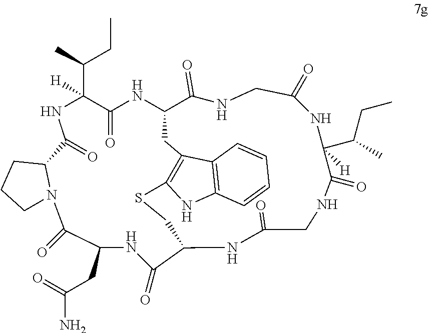 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1CCC[C@@H]1C(=O)N[C@@H]([C@@H](C)CC)C(=O)N2]

Synthesis of compound 7g was done in analogous fashion to compound 7a as disclosed above using the corresponding Fmoc-protected amino acids and performing the initial loading of 2-CTC-resin with Fmoc-D-Pro-OH. The crude product was purified using preparative HPLC to afford bicyclic octapeptide 7g (25 mg, 63%) as a white powder.

Compound 7h: HPLC-MS: Retention time Rₜ=10.75 min (Gradient B); HRMS (ESI): m/z calculated: C₄₂H₅₉N₁₀O₁₀S⁺ [M+H]⁺ 895.4131 found 895.4134.

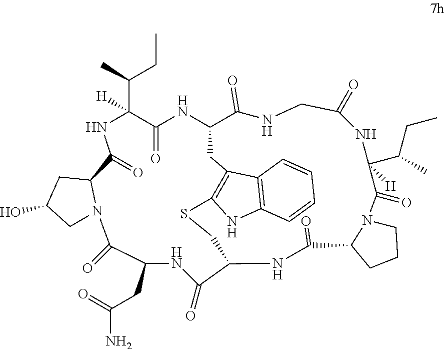 [SMILES: CC[C@H](C)[C@@H]1NC(=O)[C@@H]2C[C@@H](O)CN2C(=O)[C@H](CC(N)=O)NC(=O)[C@@H]2CSc3[nH]c4ccccc4c3C[C@H](NC1=O)C(=O)NCC(=O)N[C@@H]([C@@H](C)CC)C(=O)N1CCC[C@@H]1C(=O)N2]

Synthesis of Compound 7h:

Synthesis of compound 7h was done in analogous fashion to compound 7a as disclosed above using the corresponding Fmoc-protected amino acids. The crude product was purified using preparative HPLC to afford bicyclic octapeptide compound 7h (Ama-02) (22 mg, 64%) as a white powder.

Compound 7i: HPLC-MS: Retention time Rₜ=10.10 min (Gradient B); HRMS (ESI): m/z calculated: C₄₂H₅₉N₁₀O₁₂S⁺ [M+H]⁺ 927.4029 found 927.4031.

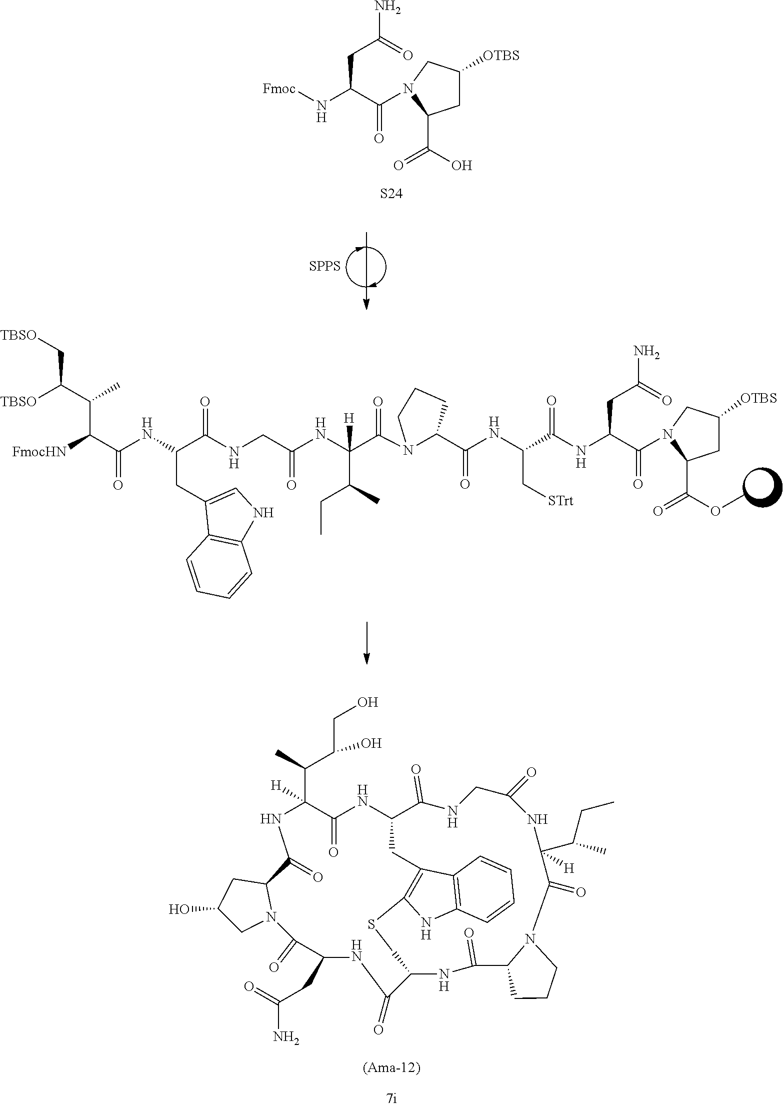

Synthesis of Compound 7i:

Starting from peptidyl resin S24, Fmoc-D-Pro-OH was used instead of Fmoc-Gly-OH at position 4 to install the linear precursor. Following the same procedure used in the synthesis of S29, compound 7i (Ama-12) was obtained as a white solid powder after RP-HPLC purification.

¹H NMR (700 MHz, DMSO-d₆) δ=11.26 (s, 1H), 8.39 (d, J=3.9 Hz, 1H), 8.24 (d, J=3.5 Hz, 1H), 8.07 (d, J=7.9 Hz, 1H), 8.04 (s, 1H), 7.93 (s, 1H), 7.91 (s, 1H), 7.58 (d, J=9.1 Hz, 1H), 7.57 (d, J=7.2 Hz, 1H), 7.40 (s, 1H), 7.25 (d, J=8.2 Hz, 1H), 7.11 (t, J=7.5 Hz, 1H), 7.02 (t, J=7.5 Hz, 1H), 5.03-4.96 (m, 1H), 4.80 (dd, J=8.0, 3.8 Hz, 1H), 4.53-4.47 (m, 1H), 4.45-4.42 (m, 1H), 4.41 (s, 1H), 4.34-4.28 (m, 2H), 4.01 (dd, J=8.0, 3.8 Hz, 1H), 3.89 (t, J=7.8 Hz, 1H), 3.84 (dd, J=10.9, 3.0 Hz, 1H), 3.74 (d, J=10.9 Hz, 1H), 3.54-3.47 (m, 2H), 3.07-3.02 (m, 2H), 2.97 (dd, J=14.7, 6.1 Hz, 1H), 2.90 (dd, J=16.2, 5.6 Hz, 1H), 2.81 (dd, J=11.0, 2.8 Hz, 1H), 2.25-2.19 (m, 2H), 1.92-1.85 (m, 2H), 1.82-1.78 (m, 1H), 1.66-1.60 (m, 1H), 1.59-1.54 (m, 1H), 1.19-1.13 (m, 1H), 0.90 (d, J=7.0 Hz, 3H), 0.85 (d, J=6.9 Hz, 3H), 0.86-0.83 (m, 1H), 0.83 (t, J=7.4 Hz, 3H).

¹³C NMR (HSQC, 176 MHz, DMSO-d₆) δ=122.7, 122.0, 120.6, 119.1, 111.6, 73.0, 71.4, 69.1, 64.0, 62.4, 60.6, 61.5, 57.3, 56.7, 56.2, 53.9, 51.0, 52.8, 55.5, 47.5, 41.9, 40.3, 38.8, 38.7, 38.3, 37.9, 36.4, 35.5, 34.5, 30.4, 29.8, 29.3, 27.2, 24.3, 25.0, 15.0, 14.3, 11.3 ppm.

Compound 7j: HPLC-MS: Retention time Rₜ=8.95 min(Gradient B); HRMS (ESI): m/z calculated: C₄₀H₅₇N₁₀O₁₂S⁺ [M+H]⁺ 901.3873 found 901.3866.

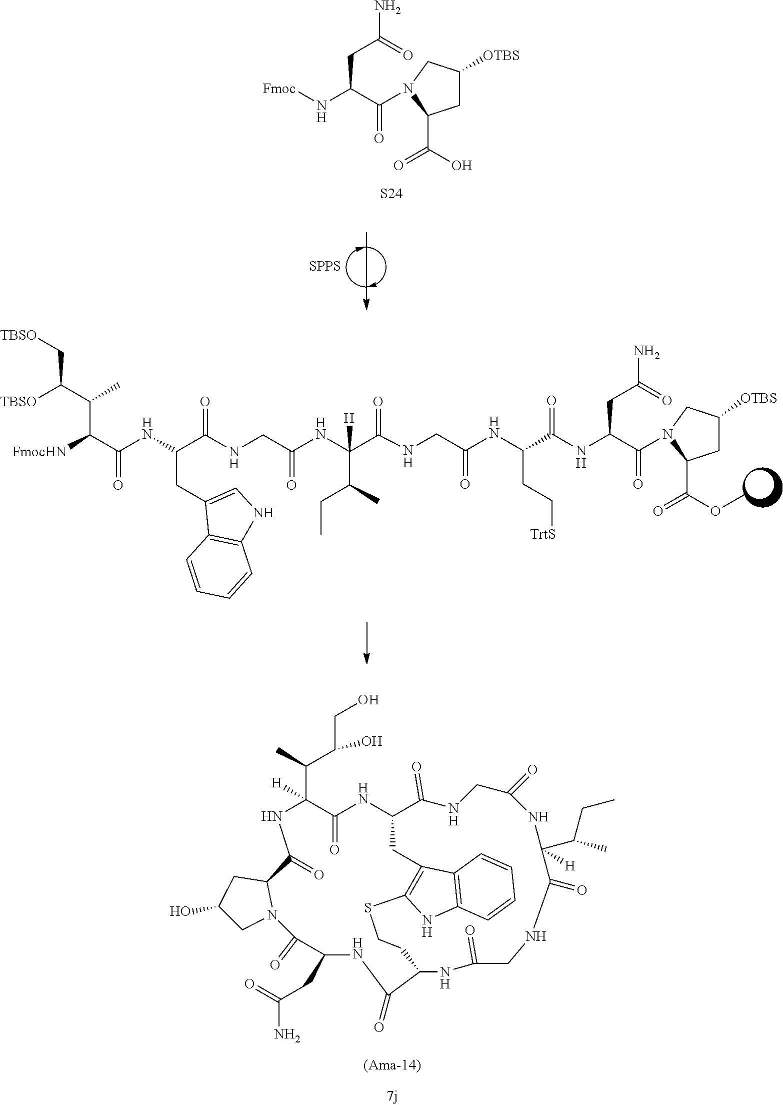

Synthesis of Compound 7:

Starting from peptidyl resin S24, Fmoc-L-HomoCys(Trt)-OH was used instead of Fmoc-L-Cys(Trt)-OH to install the linear precursor. Following the same procedure used in the synthesis of S29, compound 7j (Ama-14) was obtained as a white solid powder after RP-HPLC purification.

¹H NMR (700 MHz, DMSO-d₆) δ=11.11 (s, 1H), 9.00 (t, J=6.2 Hz, 1H), 8.42 (d, J=4.9 Hz, 1H), 8.05 (d, J=3.7 Hz, 1H), 8.02 (d, J=7.8 Hz, 1H), 8.00 (d, J=8.7 Hz, 1H), 7.78 (s, 1H), 7.52 (d, J=7.0 Hz, 1H), 7.40 (d, J=8.3 Hz, 1H), 7.38 (d, J=9.6 Hz, 1H), 7.30 (s, 1H), 7.27 (d, J=8.1 Hz, 1H), 7.12 (t, J=7.5 Hz, 1H), 7.02 (t, J=7.3 Hz, 1H), 5.26 (s, 1H), 4.81 (s, 1H), 4.66 (dd, J=10.1, 5.2 Hz, 1H), 4.40-4.30 (m, 4H), 4.27 (dd, J=10.5, 7.3 Hz, 1H), 3.86 (dd, J=17.1, 6.9 Hz, 1H), 3.81 (dd, J=8.6, 4.8 Hz, 1H), 3.78 (dd, J=10.6, 3.4 Hz, 1H), 3.68 (s, 1H), 3.66 (s, 1H), 3.57 (dd, J=10.3, 5.6 Hz, 1H), 3.45 (d, J=14.0 Hz, 1H), 3.43 (s, 1H), 3.42-3.39 (m, 2H), 2.94-2.86 (m, 2H), 2.72 (dd, J=15.1, 6.0 Hz, 1H), 2.37 (d, J=7.6 Hz, 1H), 2.21-2.14 (m, 2H), 2.01 (dd, J=15.2, 7.7 Hz, 1H), 1.91-1.85 (m, 1H), 1.78-1.76 (m, 1H), 1.67-1.63 (m, 1H), 1.61-1.56 (m, 1H), 1.48-1.41 (m, 1H), 1.21-1.15 (m, 1H), 0.92 (d, J=7.0 Hz, 3H), 0.87-0.84 (m, 4H), 0.84 (d, J=6.9 Hz, 3H).

¹³C NMR (HSQC, 176 MHz, DMSO-d₆) δ=122.6, 119.2, 118.6, 111.5, 73.1, 68.9, 64.1, 61.8, 59.3, 56.0, 55.5, 54.8, 52.1, 50.6, 43.1, 42.3, 40.3, 39.2, 38.7, 38.4, 38.2, 37.5, 35.6, 35.1, 31.4, 29.6, 29.2, 25.7, 15.6, 14.1, 11.8, 11.1 ppm

Compound 7k: HPLC-MS: Retention time Rₜ=10.24 min (Gradient B); HRMS (ESI): m/z calculated: C₄₆H₆₁N₁₀O₁₃S⁺ [M+H]⁺ 993.4135 found 993.4138.

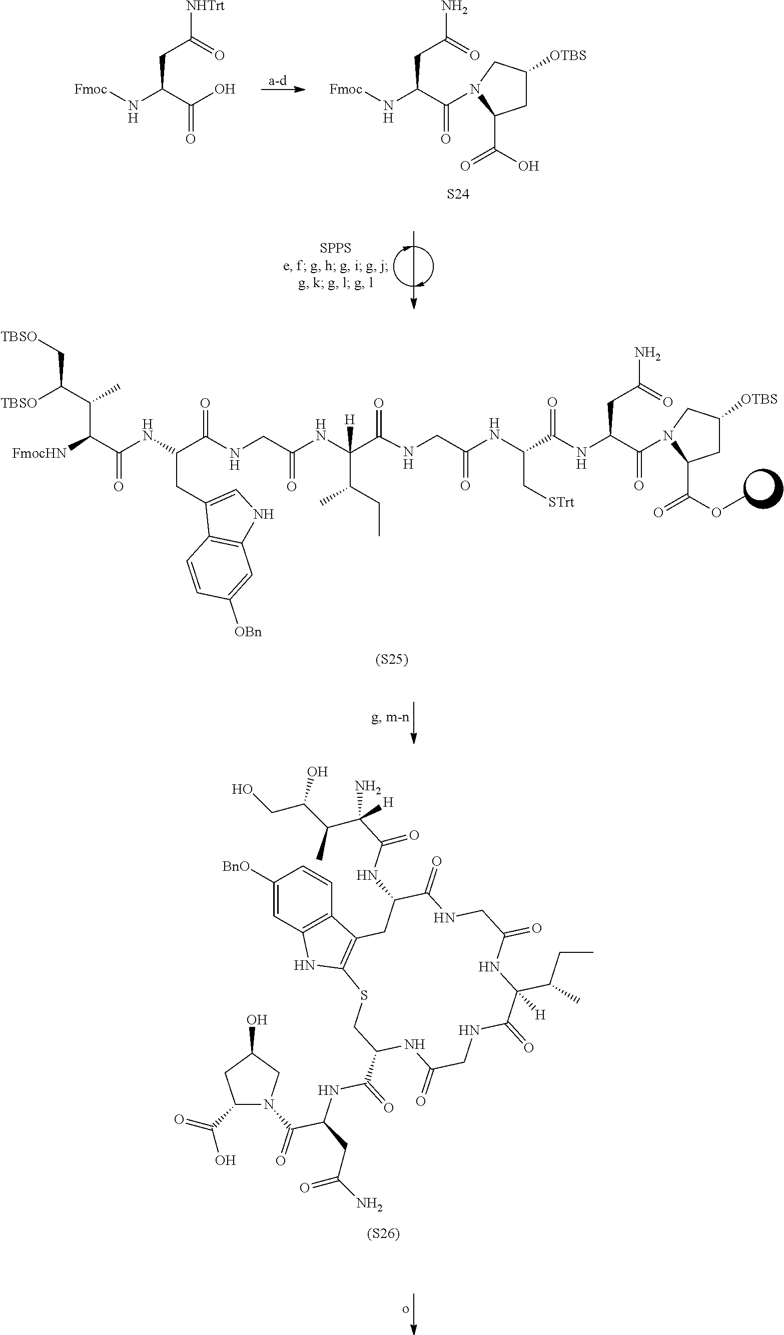

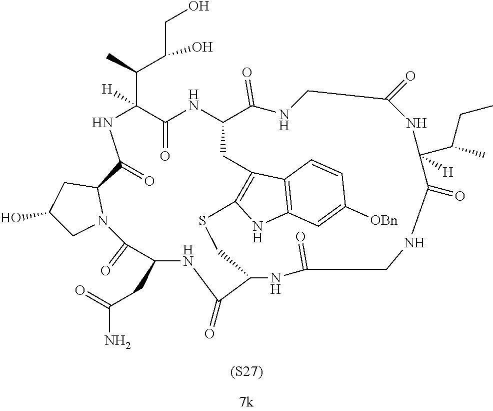 [SMILES: CC[C@H](C)[C@@H]1NC(=O)CNC(=O)[C@@H]2Cc3c([nH]c4cc(OCc5ccccc5)ccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; "(S27)"]

Synthesis of compound 7k:

a) DCC, NHS, DCM; b) H-Hyp-OH, NaHCO₃, dioxane/H₂O; c) TFA/DCM (1:1), 1 h; d) TBDMSOTf, lutidine, DCM, 16 h; e) 2-CTC-resin, DCM, DIPEA; f) MeOH/DIPEA/DCM (1:1:8); g) piperdine/DMF (1:4); h) Fmoc-L-Cys-OH, TBTU, DIPEA, DMF; h) Fmoc-Gly-OH, TBTU, DIPEA, DMF; i) Fmoc-Ile-OH, TBTU, DIPEA, DMF; j) Fmoc-Gly-OH, TBTU, DIPEA, DMF; k) Fmoc-L-6-OBn-Trp-OH (15), TBTU, DIPEA, DMF; I) Fmoc-L-(2S,3R,4R)-(TBS)₂-DHIle-OH (16), TBTU, DIPEA, DMF; m) TFE/HOAc/DCM (1:1:8); n) TBAF, THF; o) HATU, DIPEA, DMF/DCM;

Compound 7l: Retention time Rₜ=8.49 min (Gradient A); HRMS (ESI): m/z calculated: C₄₀H₅₆N₁₁O₁₂S⁺ [M+H]⁺ 901.3873, found 901.3890.

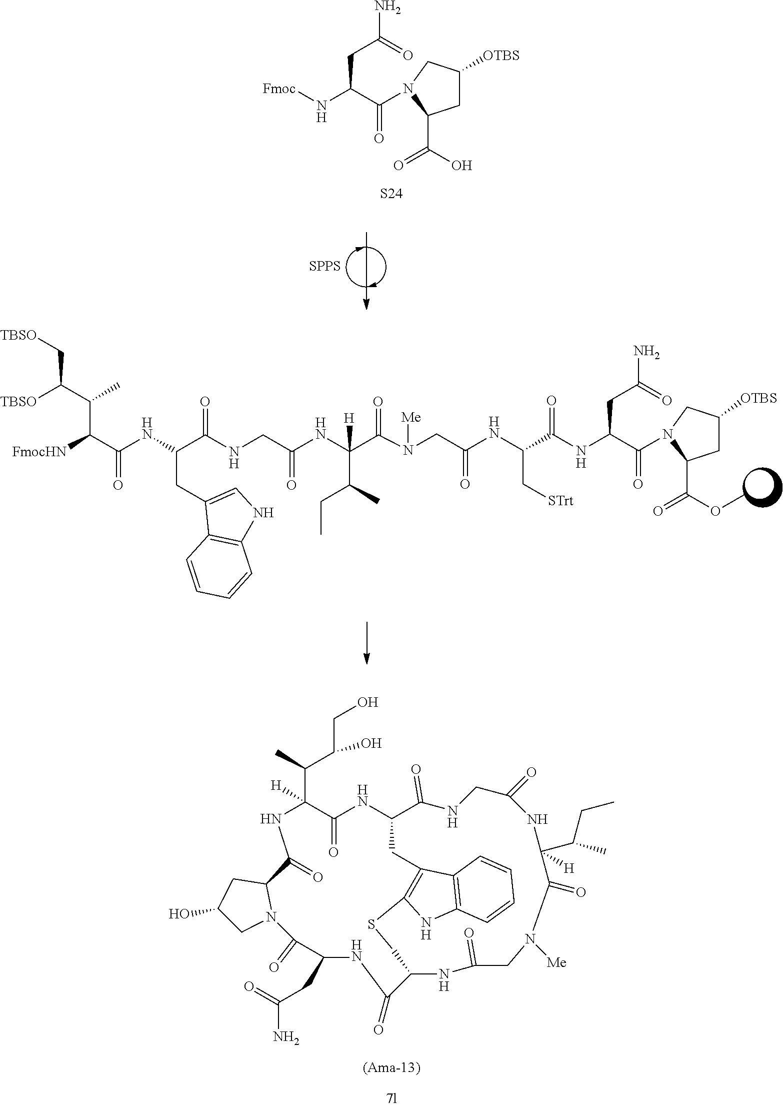

Synthesis of Compound 7l:

Starting from peptidyl resin S24, Fmoc-Sar-OH was used instead of Fmoc-Gly-OH at position 4 to install the linear precursor. Following the same procedure used in the synthesis of S29, compound 7l (Ama-13) was obtained as a white solid powder after RP-HPLC purification.

¹H NMR (700 MHz, DMSO-d₆) δ=11.25 (s, 1H), 8.51 (d, J=3.6 Hz, 1H), 8.29 (s, 1H), 8.07 (d, J=7.0 Hz, 1H), 7.98 (s, 1H), 7.93 (d, J=9.4 Hz, 1H), 7.92 (d, J=10.8 Hz, 1H), 7.61 (d, J=9.2 Hz, 1H), 7.54 (d, J=7.8 Hz, 1H), 7.37 (s, 1H), 7.25 (d, J=8.1 Hz, 1H), 7.12 (t, J=7.5 Hz, 1H), 7.02 (t, J=7.4 Hz, 1H), 5.28 (s, 1H), 4.96-4.89 (m, 1H), 4.78 (s, 1H), 4.60-4.54 (m, 1H), 4.48 (d, J=17.4 Hz, 1H), 4.42 (dd, J=10.3, 5.2 Hz, 1H), 4.41 (s, 1H), 4.31 (dd, J=11.4, 6.8 Hz, 1H), 4.28 (d, J=6.9 Hz, 1H), 4.19 (dd, J=18.6, 8.1 Hz, 1H), 3.83 (dd, J=11.3, 3.6 Hz, 1H), 3.72 (d, J=10.3 Hz, 1H), 3.55-3.51 (m, 1H), 3.44 (d, J=17.2 Hz, 1H), 3.39 (d, J=11.4 Hz, 1H), 3.24 (d, J=16.8 Hz, 1H), 3.19 (s, 3H), 3.13-3.09 (m, 2H), 2.95 (dd, J=14.3, 5.6 Hz, 1H), 2.91 (dd, J=16.4, 5.9 Hz, 1H), 2.86 (dd, J=11.0, 2.7 Hz, 1H), 2.24-2.19 (m, 2H), 2.01 (dd, J=15.4, 7.3 Hz, 1H), 1.92-1.86 (m, 1H), 1.65-1.60 (m, 2H), 1.29 (d, J=20.1 Hz, 1H), 0.91 (d, J=7.1 Hz, 3H), 0.87-0.82 (m, 7H).

¹³C NMR (HSQC, 176 MHz, DMSO-d₆) δ=130.1, 122.7, 120.3, 119.2, 111.6, 73.0, 69.1, 63.9, 62.4, 56.2, 55.5, 54.3, 52.8, 52.1, 51.0, 46.2, 37.8, 35.7, 27.1, 29.2, 14.8, 14.3, 11.4, 11.3, ppm.

Compound 7m: Retention time Rₜ=9.29 min (Gradient A); HRMS (ESI): m/z calculated: C₄₁H₅₉N₁₀O₁₂S⁺ [M+H]⁺ 915.4029, found 915.4059.

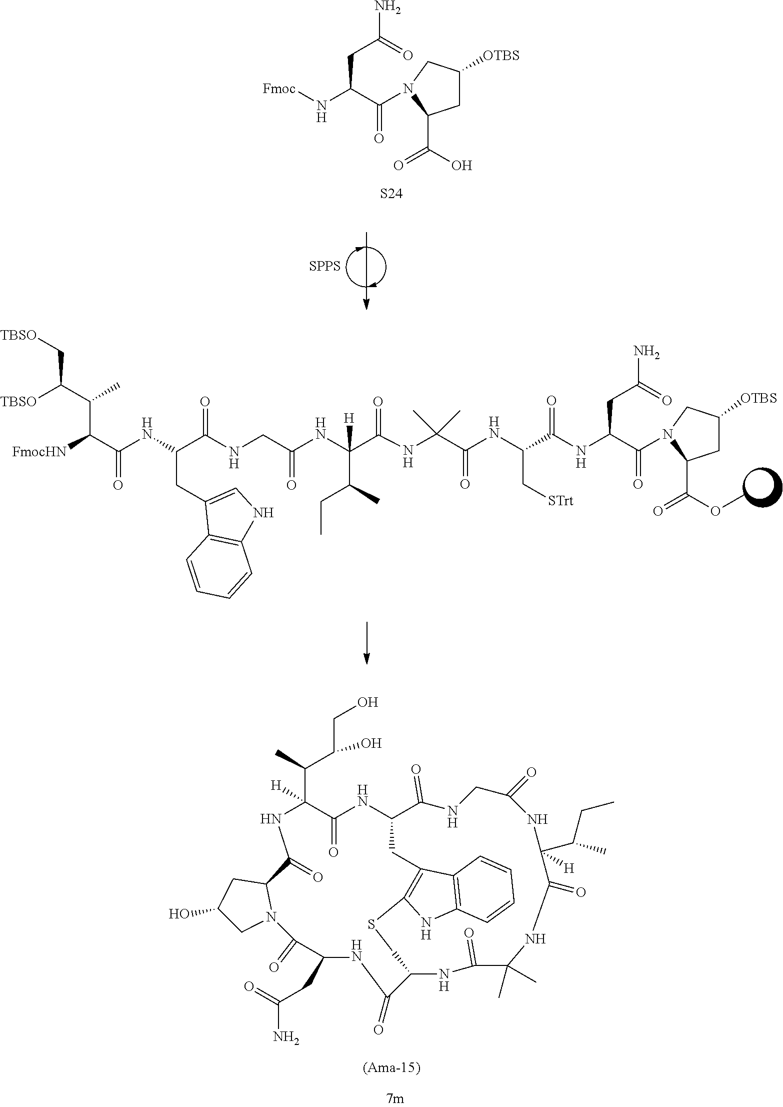

Synthesis of Compound 7m:

Starting from peptidyl resin S24, Fmoc-Aib-OH (N-α-Fmoc-α-aminoisobutyric acid) was used instead of Fmoc-Gly-OH at position 4 to install the linear precursor. Following the same procedure used in the synthesis of S29, compound 7m (Ama-15) was obtained as a white solid powder after RP-HPLC purification.

¹H NMR (500 MHz, DMSO-d₆) δ=11.24 (s, 1H), 8.38 (s, 2H), 8.29 (s, 1H), 8.18 (s, 1H), 8.14 (d, J=8.6 Hz, 1H), 7.96 (d, J=9.6 Hz, 1H), 7.91 (d, J=8.2 Hz, 1H), 7.82 (d, J=9.8 Hz, 1H), 7.60 (d, J=8.0 Hz, 1H), 7.46 (s, 1H), 7.25 (d, J=8.1 Hz, 1H), 7.11 (t, J=7.6 Hz, 1H), 7.01 (t, J=7.3 Hz, 1H), 5.20 (s, 1H), 5.10-5.00 (m, 1H), 4.76 (s, 1H), 4.47-4.41 (m, 2H), 4.40 (d, J=1.6 Hz, 1H), 4.33-4.25 (m, 2H), 3.82 (d, J=8.7 Hz, 1H), 3.75 (d, J=11.0 Hz, 1H), 3.67-6.62 (m, 1H), 3.51 (dd, J=9.8, 6.2 Hz, 1H), 3.27-3.22 (m, 2H), 3.04 (dd, J=14.8, 6.4 Hz, 1H), 2.99-2.91 (m, 2H), 2.79 (d, J=7.7 Hz, 1H), 2.26-2.16 (m, 2H), 1.87 (t, J=10.7 Hz, 1H), 1.54-1.48 (m, 2H), 1.42 (s, 3H), 1.24 (s, 3H), 1.14-1.10 (m, 1H), 0.89 (d, J=6.9 Hz, 3H), 0.85-0.79 (m, 7H).

¹³C NMR (HSQC, 126 MHz, DMSO-d₆) δ=122.7, 121.1, 119.1, 111.6, 72.8, 69.1, 63.9, 62.4, 59.0, 56.2, 55.6, 53.8, 53.4, 51.1, 41.8, 41.6, 38.4, 38.1, 35.3, 34.5, 34.6, 30.6, 28.4, 26.8, 25.9, 25.7, 22.9, 21.3, 15.3, 14.1, 11.0, ppm

Compound 7n: HPLC-MS: Retention time Rₜ=8.73 min (Gradient A); HRMS (ESI): m/z calculated C₃₈H₅₃N₁₀O₁₁S⁺ [M+H]⁺ 857.3610, found 857.3605.

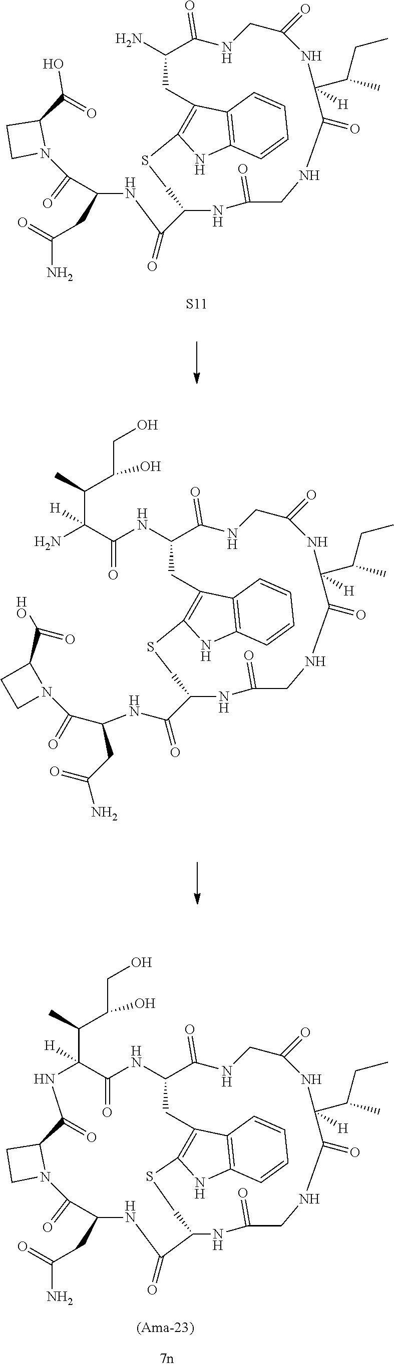

Synthesis of Compound 7n:

2-CTC resin (1 g, 0.98 mmol/g) was loaded with Fmoc-L-Aze-OH as described (cf. Material and Methods). The resin loading was determined to be 0.30 mmol/g. The Fmoc group was removed according to Method A. Fmoc-Asn(Trt)-OH (4 eq) was coupled to the deprotected resin according to Method B. Chloranil test was performed to confirm the coupling to be complete. The Fmoc group of the resulting resin was removed according to Method A. The following peptide sequences Fmoc-L-Cys(Trt)-OH (4 eq), Fmoc-Gly-OH (4 eq), Fmoc-L-Ile-OH (4 eq), Fmoc-Gly-OH (4 eq) and Fmoc-L-Trp-OH (4 eq) was coupled to the deprotected resin according to Method A and B. Following the iodine-mediated cyclisation on solid support (cf. Material and Methods) and cleavage of the peptide from the resin using condition A, 37.2 mg peptide S11 was obtained as white solid powder with 17% yield after subsequent purification. To a solution of Fmoc-DHIle(TBS)₂—OH (34 mg, 0.06 mmol, 1.10 eq), and DIPEA (25 μL, 0.14 mmol, 2.60 eq) in DMF (2 mL) was added COMU (25 mg, 0.06 mmol, 1.1 eq) and the resulted solution was stirred for 30 min at 0° C. Fully deprotected monocyclic heptapeptide S11 (40 mg, 0.055 mmol, 1.0 eq) was dissolved in DMF (1 mL) and added to the above solution. The solution was stirred for 6 h at room temperature, then diluted with DCM (50 mL) and washed with 10% citric acid (2×10 mL) and sat. NaHCO₃ (2×10 mL). The organic phase was washed with brine (2×20 mL), dried over NaSO₄ and evaporated under reduced pressure. The crude product was dissolved in MeCN (2 mL). Et₂NH (2 ml) was added and stirred for 15 min at r.t. The solvent was removed under reduced pressure and the precipitate was redissolved in THF (3 mL). Then, a solution of TBAF in THF (1 M, 1.5 mL, 10 eq) was added and the reaction mixture was stirred for 2 h at r.t. The crude peptide was precipitated in diethyl ether and redissolved in the water. After lyophilization, the crude peptide was submitted to the next step without any further purification. The crude monocyclic octapeptide was dissolved in DMF (30 mL). Then, DIPEA (2.20 eq) and HATU (2.0 eq) were added at 0° C. The reaction mixture was allowed to warm to r.t. and stirred overnight. After concentrated under reduced pressure, the crude product was purified using preparative HPLC to afford bicyclic octapeptide compound 7n (Ama-23) (16 mg, 35% yield) as a white powder.

¹H NMR (500 MHz, DMSO-d₆): δ=11.24 (s, 1H), 8.84 (t, J=6.35 Hz, 1H), 8.45 (d, J=4.28 Hz, 1H), 8.34 (d, J=3.92 Hz, 1H), 8.25 (d, J=10.69 Hz, 1H), 8.19 (bs, 1H), 8.00 (d, J=7.94 Hz, 1H), 7.88 (d, J=10.10 Hz, 1H), 7.81 (d, J=8.30 Hz, 1H), 7.57 (d, J=7.58 Hz, 1H), 7.51 (br, 1H), 7.25 (d, J=8.1 Hz, 1H), 7.11 (t, J=7.77 Hz, 1H), 7.01 (t, J=7.70 Hz, 1H), 4.95-5.04 (m, 1H), 4.75 (t, J=8.33 Hz, 1H), 4.64-4.69 (m, 1H), 4.55-4.63 (m, 2H), 4.43-4.51 (m, 1H), 4.34-4.41 (m, 1H), 4.17 (dd, J=18.74, 7.74 Hz, 1H), 3.90 (dd, J=17.65, 7.26 Hz, 1H), 3.28-3.35 (m, 2H), 3.12 (t, J=11.6 Hz, 1H), 3.00 (dd, J=14.92, 6.31 Hz, 1H), 2.89 (dd, J=15.49, 4.78 Hz, 1H), 2.78 (dd, J=10.90, 3.06 Hz, 1H), 2.58-2.66 (m, 1H), 2.26-2.34 (m, 1H), 1.50-1.63 (m, 2H), 1.06-1.17 (m, 1H), 0.92 (d, J=7.2 Hz, 3H), 0.83 (t, J=7.4 Hz, 3H), 0.80 ppm (d, J=6.7 Hz, 3H).

¹³C NMR (HSQC, 126 MHz, DMSO-d₆): δ=122.7, 120.6, 119.1, 111.6, 72.7, 64.1, 62.3, 59.3, 55.5, 53.5, 49.4, 42.6, 42.1, 41.8, 39.1, 34.8, 34.2, 30.4, 25.6, 21.2, 15.4, 14.1, 10.9 ppm

Compound 7o: HPLC-MS: Retention time Rₜ=8.80 min (Gradient A). HRMS (ESI): m/z calculated: C₄₀H₅₇N₁₀O₁₂S⁺ [M+H]⁺ 901.3873, found 901.3885.

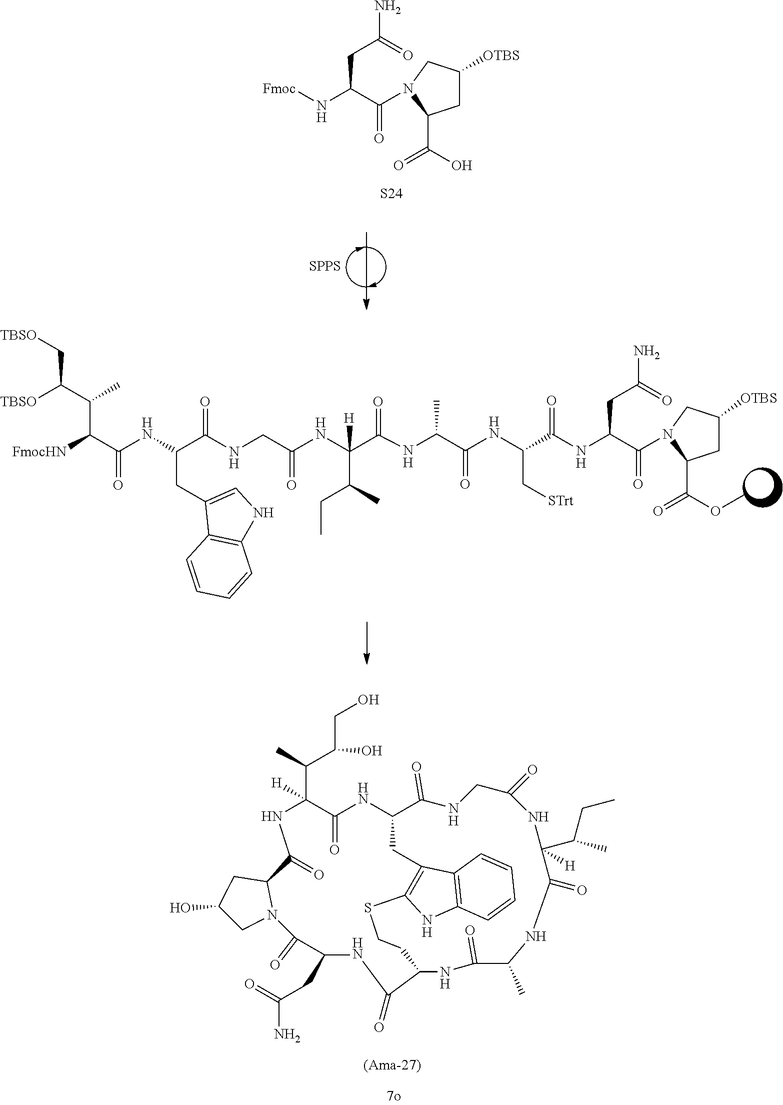

Synthesis of Compound 70:

Starting from peptidyl resin S24, Fmoc-D-Ala-OH was used instead of Fmoc-Gly-OH at position 4 to install the linear precursor. Following the same procedure used in the synthesis of S29, compound 7o (Ama-27) was obtained as a white solid powder after RP-HPLC purification.

¹H NMR (500 MHz, DMSO-d₆) δ=11.23 (s, 1H), 8.71 (d, J=7.6 Hz, 1H), 8.48 (d, J=4.6 Hz, 1H), 8.47 (d, J=3.4 Hz, 1H), 8.15 (s, 1H), 8.10 (d, J=8.0 Hz, 1H), 8.00 (d, J=9.8 Hz, 1H), 7.97 (d, J=9.3 Hz, 1H), 7.91 (d, J=8.1 Hz, 1H), 7.58 (d, J=7.8 Hz, 1H), 7.45 (s, 1H), 7.25 (d, J=8.2 Hz, 1H), 7.11 (t, J=7.2 Hz, 1H), 7.01 (t, J=7.5 Hz, 1H), 5.00-4.93 (m, 1H), 4.73 (dd, J=7.7, 3.8 Hz, 1H), 4.57-4.50 (m, 1H), 4.46 (dd, J=9.4, 5.9 Hz, 1H), 4.41 (s, 1H), 4.28 (dd, J=11.4, 6.9 Hz, 1H), 4.18 (dd, J=18.6, 8.3 Hz, 2H), 4.12-4.06 (m, 1H), 3.70 (dd, J=7.9, 3.5 Hz, 1H), 3.52 (dd, J=10.4, 6.4 Hz, 1H), 3.37 (dd, J=11.1, 3.7 Hz, 1H), 3.31 (dd, J=11.8, 4.7 Hz, 1H), 3.09 (t, J=11.4 Hz, 1H), 3.04 (dd, J=14.9, 6.4 Hz, 1H), 2.93 (dd, J=15.9, 4.6 Hz, 1H), 2.79 (dd, J=10.8, 3.3 Hz, 1H), 2.25-0.15 (m, 2H), 1.88 (td, J=12.4, 3.4 Hz, 1H), 1.59-1.53 (m, 2H), 1.24 (d, J=7.4 Hz, 4H), 1.18-1.07 (m, 1H), 0.89 (d, J=7.1 Hz, 3H), 0.84-0.82 (m, 1H), 0.83 (t, J=7.3 Hz, 3H), 0.80 (d, J=6.7 Hz, 3H).

¹³C NMR (HSQC, 126 MHz, DMSO-d₆) δ=122.7, 120.8, 119.1, 111.6, 72.8, 69.1, 63.9, 62.4, 59.3, 56.3, 56.2, 55.6, 55.5, 53.8, 53.1, 51.1, 49.2, 41.8, 41.7, 40.3, 38.6, 38.3, 38.1, 37.9, 34.8, 34.6, 30.6, 30.5, 25.7, 25.6, 25.5, 17.5, 15.2 14.1, 10.9, ppm

Compound 7p: HPLC-MS: Retention time Rₜ=7.98 min (Gradient A); HRMS (ESI): m/z calculated: C₃₉H₅₅N₁₀O₁₂S⁺ [M+H]⁺ 887.3716, found 887.3726.

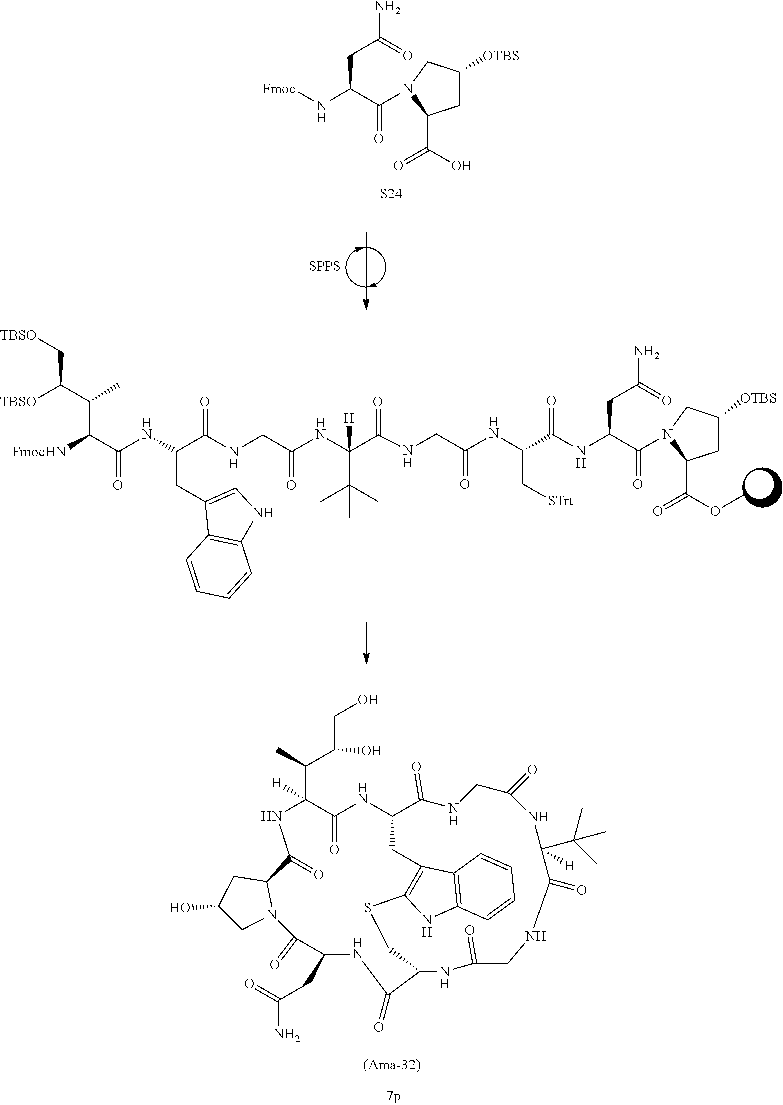

Synthesis of Compound 7p:

Starting from peptidyl resin S24, Fmoc-L-Tle-OH was used instead of Fmoc-L-Ile-OH at position 3 to install the linear precursor. Following the same procedure used in the synthesis of S29, compound 7p (Ama-32) was obtained as a white solid powder after RP-HPLC purification.

¹H NMR (500 MHz, DMSO-d₆) δ=11.22 (s, 1H), 8.75 (t, J=6.0 Hz, 1H), 8.43 (d, J=3.6 Hz, 1H), 8.19 (d, J=4.3 Hz, 1H), 8.14 (s, 1H), 8.09 (d, J=9.4 Hz, 1H), 8.03 (d, J=9.8 Hz, 1H), 7.96 (d, J=9.4 Hz, 1H), 7.91 (d, J=8.1 Hz, 1H), 7.58 (d, J=7.8 Hz, 1H), 7.45 (s, 1H), 7.26 (d, J=8.1 Hz, 1H), 7.12 (t, J=7.1 Hz, 1H), 7.02 (t, J=7.5 Hz, 1H), 5.00-4.93 (m, 1H), 4.71 (dd, J=7.7, 3.9 Hz, 1H), 4.71 (dd, J=7.7, 3.9 Hz, 1H), 4.61-4.54 (m, 1H), 4.45 (dd, J=9.3, 5.9 Hz, 1H), 4.41 (s, 1H), 4.31-4.24 (m, 3H), 3.92 (dd, J=17.3, 7.4 Hz, 1H), 3.83-3.78 (m, 1H), 3.76-3.72 (m, 2H), 3.52 (dd, J=10.4, 6.5 Hz, 1H), 3.45 (d, J=17.5 Hz, 1H), 3.42-3.28 (m, 3H), 3.09 (t, J=11.4 Hz, 1H), 3.04 (dd, J=14.7, 6.2 Hz, 1H), 2.94 (dd, J=15.8, 4.5 Hz, 1H), 2.78 (dd, J=10.8, 3.4 Hz, 1H), 2.23-2.16 (m, 2H), 1.88 (td, J=12.5, 3.4 Hz, 1H), 0.99-0.95 (m, 1H), 0.93 (s, 9H), 0.90 (d, J=7.1 Hz, 3H).

¹³C NMR (HSQC, 126 MHz, DMSO-d₆) δ=122.6, 120.8, 119.1, 111.5, 72.8, 69.1, 64.0, 63.8, 63.6, 62.4, 56.2, 55.6, 53.9, 52.8, 50.9, 42.9, 42.0, 41.9, 39.0, 38.9, 36.2, 34.7, 30.4, 29.5, 26.9, 25.3, 14.1 ppm

Compound 7q: HPLC-MS: Retention time Rₜ=9.03 min (Gradient A). HRMS (ESI): m/z calculated: C₃₉H₅₃F₂N₁₀O₁₁S⁺ [M+H]⁺ 907.3579, found 907.3572.

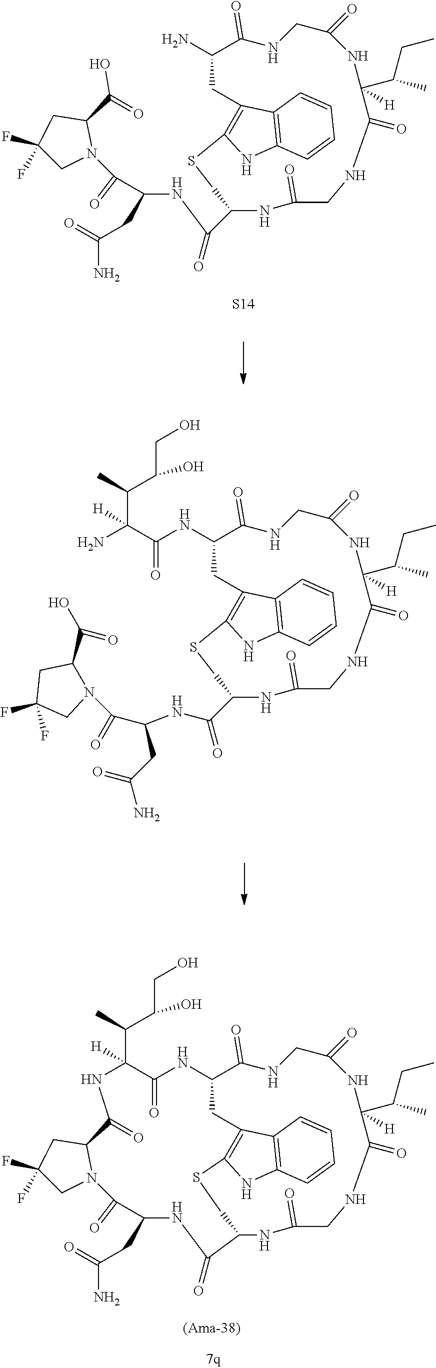

Synthesis of Compound 7q:

According to the synthesis of S11, but Fmoc-L-4-Pro(F₂)—OH was used instead of Fmoc-L-Aze-OH to assemble the linear peptide. Following the iodine-mediated cyclisation on solid support and cleavage the peptide from the resin using condition A, 37.4 mg peptide S14 was obtained as white solid powder with 16% yield after subsequent purification.

To a solution of Fmoc-DHIle(TBS)₂—OH (30 mg, 0.053 mmol, 1.10 eq), and DIPEA (22 μL, 2.6 eq) in DMF (2 mL) was added COMU (22 mg, 1.1 eq) and the resulting solution was stirred for 30 min at 0° C. Fully deprotected monocyclic heptapeptide S14 (37 mg, 0.048 mmol, 1.0 eq) was dissolved in DMF (1 mL) and added to the above solution. The solution was stirred for 6 h at room temperature, then diluted with DCM (50 mL) and washed with 10% citric acid (2×10 mL) and sat. NaHCO₃ (2×10 mL). The organic phase was washed with brine (2×20 mL), dried over NaSO₄ and evaporated under reduced pressure. The crude product was dissolved in MeCN (2 mL). Et₂NH (2 ml) was added and stirred for 15 min at r.t. The solvent was removed under reduced pressure and the precipitate was redissolved in THF (3 mL). Then, a solution of TBAF in THF (1 M, 1.5 mL, 10 eq) was added and the reaction mixture was stirred for 2 h at r.t. The crude peptide was precipitated in diethyl ether and redissolved in the water. After lyophilization, the crude peptide was submitted to the next step without any further purification. The crude monocyclic octapeptide was dissolved in DMF (30 mL). Then, DIPEA (2.20 eq) and HATU (2.0 eq) was added at 0° C. The reaction mixture was allowed to warm to r.t. and stirred overnight. After concentrated under reduced pressure, the crude product was purified using preparative HPLC to afford bicyclic octapeptide compound 7q (Ama-38) (16 mg, 37% yield) as a white powder.

¹H NMR (500 MHz, DMSO-d₆) δ=11.22 (s, 1H), 8.85 (t, J=5.4 Hz, 1H), 8.45 (d, J=4.4 Hz, 1H), 8.41 (d, J=3.0 Hz, 1H), 8.16 (s, 1H), 8.06 (d, J=9.2 Hz, 1H), 7.97 (d, J=10.2 Hz, 1H), 7.89 (t, J=9.1 Hz, 2H), 7.57 (d, J=8.3 Hz, 1H), 7.47 (s, 1H), 7.25 (d, J=8.2 Hz, 1H), 7.11 (t, J=7.6 Hz, 1H), 7.01 (t, J=7.3 Hz, 1H), 4.95-4.87 (m, 1H), 4.83 (q, J=5.0 Hz, 1H), 4.61-4.54 (m, 1H), 4.47 (dd, J=9.1, 5.9 Hz, 1H), 4.45-4.41 (m, 1H), 4.23 (dd, J=18.3, 8.8 Hz, 1H), 3.90 (dd, J=17.3, 7.4 Hz, 1H), 3.70 (dd, J=8.3, 4.0 Hz, 1H), 3.56 (dd, J=10.8, 6.5 Hz, 1H), 3.47-3.41 (m, 2H), 3.09 (t, J=11.4 Hz, 1H), 3.06-3.00 (m, 2H), 2.96 (d, J=11.4 Hz, 1H), 2.80 (d, J=7.5 Hz, 1H), 2.21-2.15 (m, 1H), 1.61-1.53 (m, 3H), 1.14-1.08 (m, 1H), 0.91 (d, J=7.0 Hz, 3H), 0.83 (t, J=7.3 Hz, 4H), 0.80 (d, J=6.8 Hz, 3H).

¹³C NMR (HSQC, 126 MHz, DMSO-d₆) δ=122.7, 120.7, 119.2, 111.5, 72.7, 64.0, 64.0, 61.2, 59.3, 55.8, 52.9, 52.8, 51.0, 38.4, 35.2, 29.3, 13.9, 15.3, 11.2 ppm

Compound 7r: HPLC-MS: Retention time Rₜ=8.64 min (Gradient A); HRMS (ESI): m/z calculated: C₄₁H₅₇N₁₀O₁₂S⁺ [M+H]⁺ 913.3873, found 913.3883.

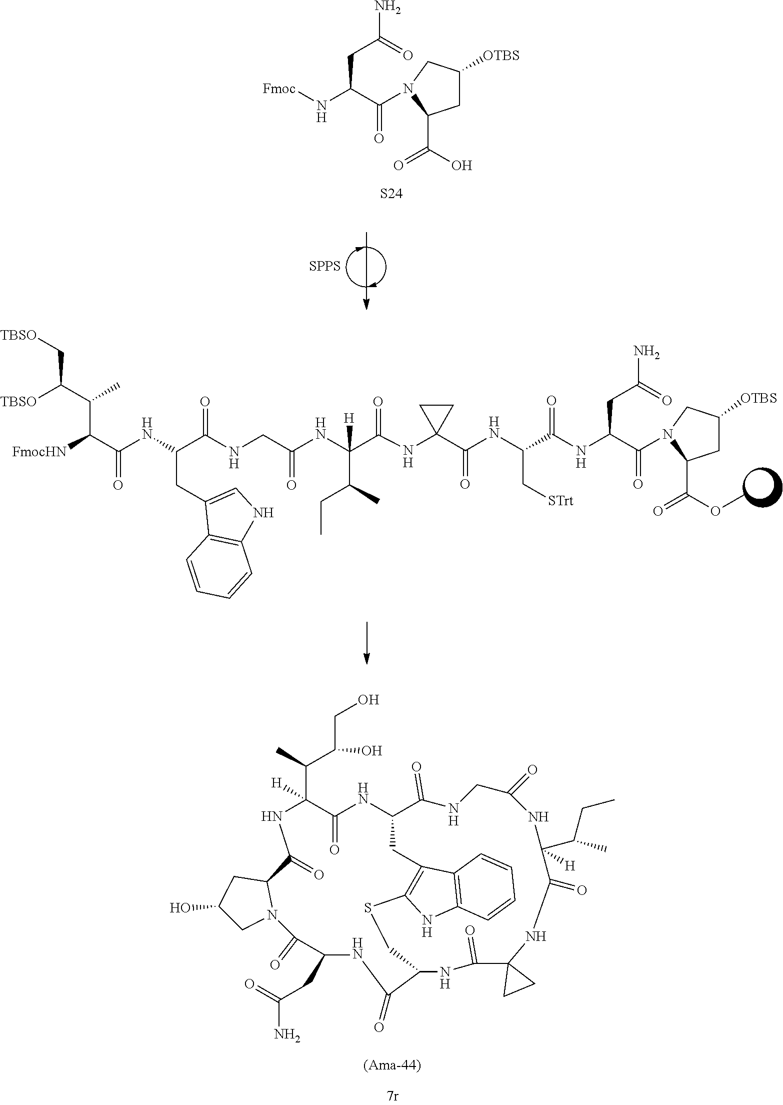

Synthesis of Compound 7r:

Starting from peptidyl resin S24, Fmoc-Acc-OH was used instead of Fmoc-Gly-OH at position 4 to install the linear precursor. Following the same procedure used in the synthesis of S29, compound 7r (Ama-44) was obtained as a white solid powder after RP-HPLC purification.

¹H NMR (500 MHz, DMSO-d₆) δ=11.22 (s, 1H), 8.98 (s, 1H), 8.47 (d, J=3.7 Hz, 1H), 8.44 (d, J=3.4 Hz, 1H), 8.24 (d, J=10.1 Hz, 1H), 8.14 (d, J=3.7 Hz, 1H), 8.12 (s, 1H), 7.96 (d, J=9.4 Hz, 1H), 7.91 (d, J=8.1 Hz, 1H), 7.59 (d, J=7.9 Hz, 1H), 7.45 (s, 1H), 7.26 (d, J=8.1 Hz, 1H), 7.11 (t, J=7.4 Hz, 1H), 7.02 (t, J=7.5 Hz, 1H), 4.97 (m, 1H), 4.75-4.67 (m, 1H), 4.64-4.54 (m, 1H), 4.46 (dd, J=9.4, 5.9 Hz, 1H), 4.42-4.37 (m, 1H), 4.30 (dd, J=11.4, 6.9 Hz, 1H), 4.23 (dd, J=18.7, 8.3 Hz, 1H), 4.02-3.97 (m, 1H), 3.60 (dd, J=8.8, 4.2 Hz, 1H), 3.52 (dd, J=10.5, 6.4 Hz, 1H), 3.43-3.27 (m, 3H), 3.08 (dd, J=20.7, 9.1 Hz, 1H), 2.94 (dd, J=15.8, 4.6 Hz, 1H), 2.79 (dd, J=13.3, 5.8 Hz, 1H), 2.26-2.15 (m, 2H), 1.94-1.84 (m, 1H), 1.58-1.48 (m, 2H), 1.44 (dt, J=26.2, 9.6 Hz, 1H), 1.07 (m, 2H), 0.90 (d, J=7.0 Hz, 3H), 0.85-0.75 (m, 2H), 0.81 (t, J=7.3 Hz, 3H), 0.71 (d, J=6.7 Hz, 3H).

¹³C NMR (HSQC, 126 MHz, DMSO-d₆) δ=122.7, 120.8, 119.1, 111.6, 72.8, 69.1, 63.9, 62.4, 59.2, 56.3, 56.2, 55.5, 53.9, 53.4, 51.0, 41.8, 41.8, 38.9, 38.8, 38.1, 38.0, 36.5, 34.6, 30.6, 35.0, 29.5, 25.9, 25.8, 17.9, 15.1, 14.0, 10.9 ppm

Precursor Synthesis

Synthesis of Dipeptide S24

### (2S,4R)-1-((((9H-fluoren-9-yl)methoxy)carbonyl)-L-asparaginyl)-4-hydroxypyrrolidine-2-carboxylic acid

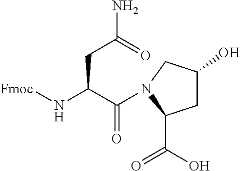

Fmoc-Asn(Trt)-OH (6 g, 10 mmol) and N-hydroxysuccinimide (NHS, 1.15 g, 10 mmol) were added to dry DMF (25 mL) at 0° C. At this temperature, the DCC (2.16 g, 10.5 mmol) was slowly added, whereby a white precipitate was formed. The reaction mixture was warmed to room temperature, and stirred for 12 h. The reaction was poured onto ice water (20 mL), extracted twice with EtOAc and the organic extracts were washed with brine, dried (Na₂SO₄) and concentrated under reduced pressure to give the crude product NHS ester of Fmoc-Asn(Trt)-OH without further purification. The crude product was dissolved in dry THF (30 mL) and treated at room temperature with DIPEA (1.8 ml, 10 mmol) and trans-L-4-hydroxylproline (1.3 g, 10 mmol). After stirring for 12 h, THF and DIPEA were evaporated and subsequent deprotection of Trt group was performed in 50% TFA in DCM (30 ml). After removal of TFA, the crude product was purified by silica-gel chromatography (DCM/MeOH) to afford dipeptide as a white solid.

¹H NMR (500 MHz, DMSO-d₆) δ=12.44 (s, 1H), 7.89 (d, J=7.5 Hz, 3H), 7.72 (d, J=7.4 Hz, 2H), 7.62 (d, J=8.2 Hz, 1H), 7.42 (t, J=7.2 Hz, 2H), 7.43-7.38 (m, 2H), 7.36-7.32 (m, 3H), 7.30 (s, 2H), 6.94 (s, 1H), 4.67 (dd, J=13.7, 7.8 Hz, 1H), 4.38-4.33 (m, 1H), 4.31-4.16 (m, 3H), 3.67 (dd, J=10.4, 4.6 Hz, 1H), 3.58-3.51 (m, 1H), 3.48-3.29 (m, 2H), 2.42-2.37 (m, 2H), 2.13-2.06 (m, 1H), 1.95-1.88 (m, 1H).

¹³C NMR (126 MHz, DMSO-d₆) δ=173.6, 171.3, 170.7, 156.2, 144.3, 141.2, 128.6, 128.1, 127.6, 125.8, 120.6, 69.3, 66.2, 58.2, 54.9, 50.0, 47.1, 40.3, 37.5, 37.1 ppm.

HRMS (ESI): m/z calculated: C₂₄H₂₆N₃O₇⁺ [M+H]⁺ 468.1765, found 468.1767.

HPLC-MS: Retention time Rₜ=6.22 min (Gradient A).

### S24: (2S,4R)-1-((((9H-fluoren-9-yl)methoxy)carbonyl)-L-asparaginyl)-4-((tert-butyldimethylsilyl)oxy) pyrrolidine-2-carboxylic acid

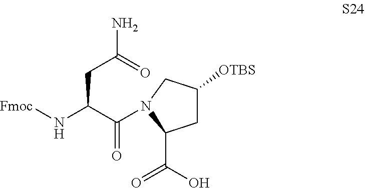

The dipeptide obtained above (2 g, 4.2 mmol) and DIPEA (1.5 ml, 8.5 mmol) were added to dry DMF (25 mL) at 0° C. At this temperature, the tert-butyldimethylsilyltrifluormethansulfonat (1.95 ml, 8.5 mmol) was slowly added. The reaction mixture was warmed to room temperature, and stirred for 12 h. The reaction was poured onto ice water (20 mL), extracted twice with EtOAc and the organic extracts were washed with brine, dried (Na₂SO₄) and concentrated under reduced pressure to give the crude product. Orthogonally protected S24 was obtained by silica-gel chromatography (DCM/MeOH).

¹H NMR (500 MHz, MeOD-d₄) δ=7.68 (d, J=7.5 Hz, 2H), 7.54 (dd, J=7.5, 3.4 Hz, 2H), 7.28 (t, J=7.4 Hz, 2H), 7.21 (t, J=7.1 Hz, 2H), 4.50 (s, 1H), 4.40 (s, 1H), 4.24-4.15 (m, 2H), 4.10 (d, J=7.0 Hz, 1H), 3.83-3.69 (m, 1H), 3.23 (dt, J=3.3, 1.6 Hz, 1H), 2.60 (s, 1H), 2.48 (d, J=8.4 Hz, 1H), 2.12 (s, 1H), 1.99 (s, 1H), 0.77 (s, 9H), −0.00 (s, 6H).

¹³C NMR (126 MHz, MeOD-d₄) δ=173.8, 156.6, 143.9, 143.8, 141.2, 127.4, 126.8, 124.9, 119.5, 70.8, 66.8, 55.1, 49.8, 46.9, 37.9, 24.8, 19.4, 17.4, −6.2 ppm.

HRMS (ESI): m/z calculated: C₃₀H₃₉N₃O₇Si⁺ [M+H]⁺ 582.2630, found 582.2632.

HPLC-MS: Retention time Rₜ=9.90 min (Gradient A).

Synthesis of Trp-OH

As the key building block of amanitin as well as limited commercial availability, the synthesis Fmoc-L-Trp(6-OBn)-OH 15 was highly required. Vilsmeier-Haack formylation of the indolic carbamates was performed to form aldehyde, which were subsequently converted into the Boc protected indole 9. Followed Horner-Wadsworth olefination was performed to form dehydro amino acid 10. The protected 6-OH-Trp derivative 10 was stereoselectively hydrogenated using with Rh(COD)₂BF₄ and (R)-MonoPhos as a ligand in excellent yield (95%) and 99% ee. After hydrolysis to 13, and Boc deprotection to 14, the protected 6-OH-Trp 15 was obtained followed by Fmoc-protection.

1) Horner-Wadsworth Olefination

For this step, the (Z)-didehydroamino acid was synthesized by using Boc-2-phosphonoglycine-methyl dimethyl ester and DBU. There is an alternative method using tetramethylguanidine (CAS 80-70-6) as base in THF at −70° C. to r.t.

2) Hydrogenation

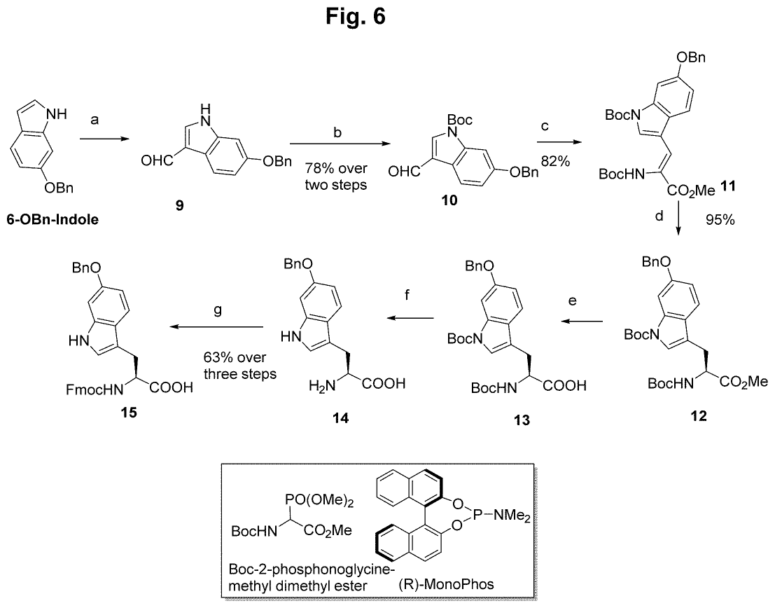

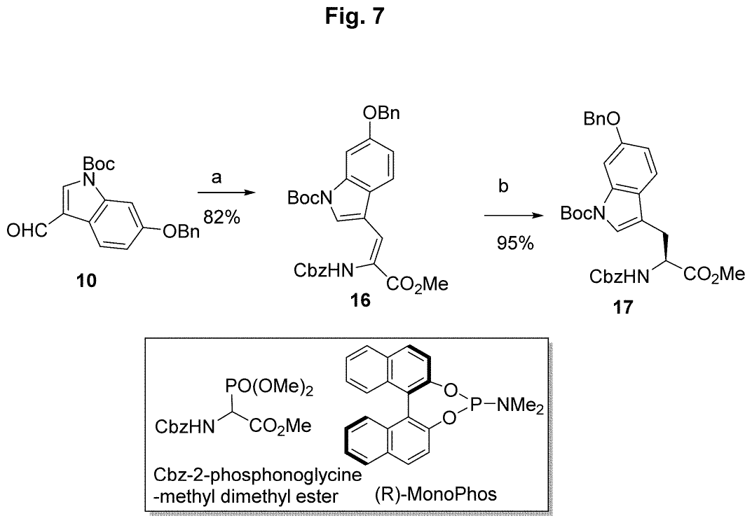

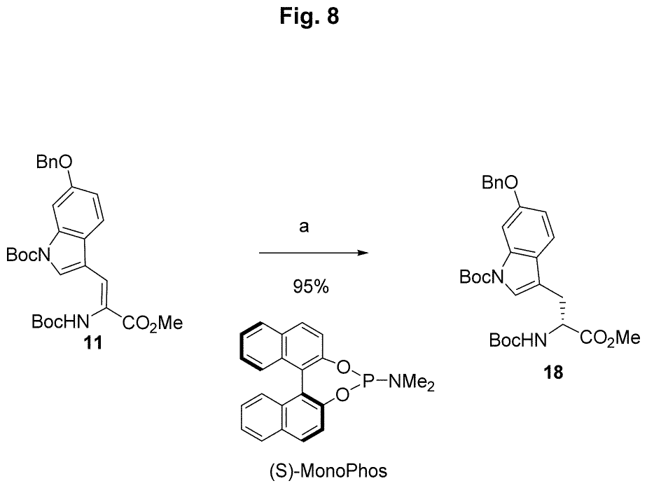

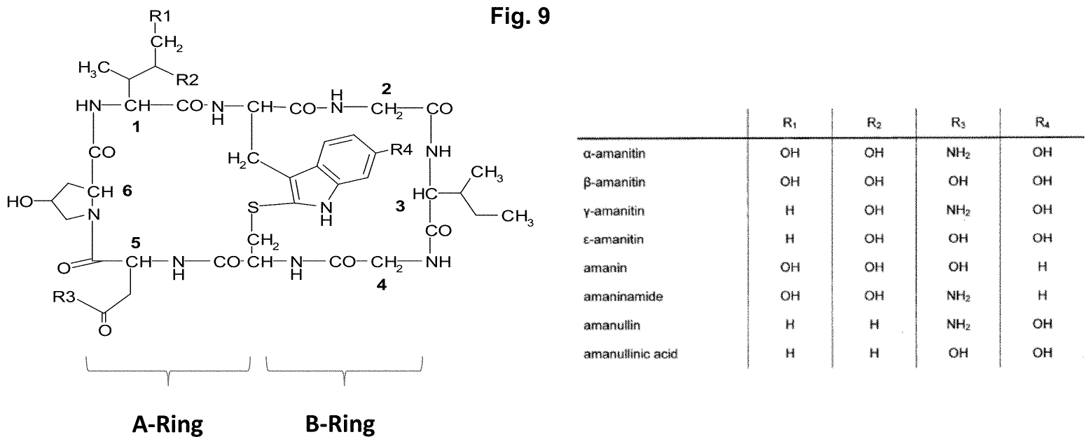

For this step, the (S)-amino acid was obtained in 99% ee by using with Rh(COD)₂BF₄ and (R)-MonoPhos as a ligand. The (R)-amino acid also was obtained in 99% ee by using with Rh(COD)₂BF₄ and (S)-MonoPhos as a ligand (see FIG. 6).

Synthesis of Aldehyde 10 Phosphorous oxychloride (1.5 mL, 15 mmol) was added dropwise to dry DMF (5 mL) at 0° C. At this temperature, the 6-OBn indole (2.23 g, 10 mmol) in dry DMF (5 mL) was slowly added, whereby a bright-yellow precipitate was formed. The reaction mixture was warmed to 45° C., and stirred for 2 h. The reaction was poured onto ice water (20 mL), extracted twice with diethyl ether and the ethereal extracts discarded. The aqueous layer was then treated with aqueous sodium hydroxide until the solution was basic and extracted with diethyl ether. The organic extracts were washed with brine, dried (Na₂SO₄) and concentrated under reduced pressure to give the crude product 9 without further purification. The crude product was dissolved in dichloromethane (10 mL) and treated at room temperature with DMAP (120 mg, 1 mmol) and di-tert-butyl dicarbonate (2.64 g, 12 mmol). After stirring for 1 h, 1 N HCl solution (10 mL) was added and dichloromethane was evaporated. The aqueous layer was extracted with several portions of diethyl ether and the combined organic extracts were washed with 1 N HCl, water, 1 N NaHCO₃, and brine. The organic layer was dried (Na₂SO₄) and concentrated under reduced pressure to give aldehyde 10 (2.73 g, 7.8 mmol, 78% over two steps) as a white solid.

Synthesis of Dehydro Amino Acid 11

To a solution of schmidt's phosphonoglycinate (Boc-2-phosphonoglycine-methyl dimethyl ester) (3.04 g, 9.1 mmol) in dichloromethane (10 mL) was added DBU (1.15 mL, 7.7 mmol). After 10 minutes stirring, aldehyde 10 (2.45 g, 7 mmol) in dichloromethane (10 mL) was added slowly. After the reaction mixture was stirred for 16 h, the solvent was evaporated under reduced pressure. The residue was dissolved in ethyl acetate (40 mL). Then the organic solution was washed with 1 N HCl (2×10 mL) and brine, dried (NaSO4), and concentrated. The crude product was purified by flash chromatography (ethyl acetate/hexanes: 1/5) to give a yellow solid (3 g, 5.74 mmol, 82%)

Hydrogenation for Synthesis of Amino Acid 12

To a solution of 11 (1.04 g, 2 mmol) in dry, degassed DCM (10 mL) was added a solution of 16 mg (0.04 mmol) [Rh(COD)₂BF₄] and 27.9 mg (0.08 mmol) (R)-MonoPhos® in dry, degassed DCM (10 mL) under argon atmosphere. The resulting mixture was placed in an autoclave and argon was replaced by H₂ (20 bar). After stirring for 3 h at room temperature, TLC showed complete conversion and the solvent was removed under reduced pressure to give the amino acid 12 (0.996 g, 1.9 mmol, 95%) as a colorless solid.

Synthesis of Fmoc-L-Trp(6-OBn)-OH 15

To a solution of methyl ester 12 (1.07 g, 2.04 mmol) in MeOH (4 mL) and THF (4 ml), 0.5 M aq. LiOH (4 ml) was added at 0° C. The mixture was allowed to warm up to room temperature for 2 h and the solvent was removed in vacuo after TLC showed full consumption of the starting material. The residue was dissolved in water and washed with DCM. Then, the aqueous layer was acidified, using 1 M aq. KHSO₄, followed by extraction with DCM (3×30 ml). The resulting organic layers were combined, dried (Na₂SO₄) and the solvent was removed. The free acid 13 (1.04 g, 2.04 mmol, quant.) was obtained as a colorless solid, which was used without further purification. The resulted product was dissolved in DCM (15 ml) at 0° C. followed adding trifluoroacetic acid (15 ml). The reaction mixture was stirred for 1 h at room temperature. The solution was concentrated and the resulting deprotected tryptophan 14 was used directly in the next step. The crude product was dissolved directly in NaHCO₃ aqueous solution (15 ml). Then Fmoc-OSu (704 mg, 2.09 mmol, 1.02 eq) in dioxane (15 ml) was added to the solution in the 0° C. The resulting solution was stirred at room temperature for 5 h and then concentrated under reduced pressure, diluted with H₂O (20 ml), acidified to pH 4 with 1 N hydrochloric acid aqueous solution, and extracted with EtOAc (2×40 mL). The organic layers were combined and washed sequentially with H₂O (2×40 mL) and saturated NaCl (10 mL) solutions, dried over anhydrous Na₂SO₄, filtered and concentrated. The crude product was purified by silica-gel chromatography (Hexanes/EtOAc, 5:1) to afford 15 as a colorless oil (683 mg, 63% over three steps).

To test different schmidt's phosphonoglycinate, the synthesis of Cbz protected 6-OBn-Trp was carried out as well.

Synthesis of Dehydro Amino Acid 16

To a solution of schmidt's phosphonoglycinate (Cbz-2-phosphonoglycine-methyl dimethyl ester) (3.01 g, 9.1 mmol) in dichloromethane (10 mL) was added DBU (1.15 mL, 7.7 mmol). After 10 minutes stirring, aldehyde 10 (2.45 g, 7 mmol) in dichloromethane (10 mL) was added slowly. After the reaction mixture was stirred for 16 h, the solvent was evaporated under reduced pressure. The residue was dissolved in ethyl acetate (40 mL). Then the organic solution was washed with 1 N HCl (2×10 mL) and brine, dried (NaSO4), and concentrated. The crude product was purified by flash chromatography (ethyl acetate/hexanes: 1/5) to give a yellow solid (3.2 g, 5.74 mmol, 82%)

Synthesis of Amino Acid 17

To a solution of 16 (1.11 g, 2 mmol) in dry, degassed DCM (10 mL) was added a solution of 16 mg (0.04 mmol) [Rh(COD)₂BF₄] and 27.9 mg (0.08 mmol) (R)-MonoPhos® in dry, degassed DCM (10 mL) under argon atmosphere. The resulting mixture was placed in an autoclave and argon was replaced by H₂ (20 bar). After stirring for 3 h at room temperature, TLC showed complete conversion and the solvent was removed under reduced pressure to give the amino acid 17 (1.06 g, 1.9 mmol, 95%) as a colorless solid.

Hydrogenation for Synthesis of Amino Acid 18

To a solution of 11 (1.04 g, 2 mmol) in dry, degassed DCM (10 mL) was added a solution of 16 mg (0.04 mmol) [Rh(COD)₂BF₄] and 27.9 mg (0.08 mmol) (S)-MonoPhos® in dry, degassed DCM (10 mL) under argon atmosphere. The resulting mixture was placed in an autoclave and argon was replaced by H₂ (20 bar). After stirring for 3 h at room temperature, TLC showed complete conversion and the solvent was removed under reduced pressure to give the amino acid 12 (0.996 g, 1.9 mmol, 95%) as a colorless solid.

Materials and Methods

HPLC-MS:

HPLC-HRMS spectra were recorded on a QTrap LTQ XL (Thermo Fisher Scientific, Waltham, Mass., USA) hyphenated to an Agilent 1200 Series HPLC-System (Agilent Technologies, Waldbronn, Germany) equipped with a C18 column (50×2 mm, particle size 3 μm). HPLC-HRMS chromatograms were obtained with a solvent gradient of 0.1% formic acid in water (Solvent A) and 0.1% formic acid in acetonitrile (Solvent B).

The solvent gradients were either gradient A, gradient B or gradient C:

Gradient A: 0-10 min 10%-50% B, 10-13 min 100% B, 13-16 min 20% B,

Gradient B: 0-10 min 20%-100% B, 10-13 min 100% B, 13-16 min 20% B.

Gradient C: 0-10 min 50%-100% B, 10-13 min 100% B, 13-16 min 20% B.

Preparation of Linear Peptides by Solid Phase Peptide (SPPS) Synthesis

Loading of Resin: 2-CTC resin (1 g, 0.98 mmol/g) was pre-swolled for 20 min in DCM in a solid phase peptide synthesis vessel (10 mL) containing a frit. After the solvent was drained, the Fmoc-amino acid (chosen according to the amino acid sequence) Fmoc-Axx-OH or dipeptide Fmoc-Asn-Hyp(OTBS)—OH (0.3 mmol) and DIPEA (4.5 mmol) in DCM (3 mL) were added to the resin. The mixture was agitated for 2 h and then the solvent was drained. The resin was washed with DMF (4×3 mL). Then a mixture of MeOH/DIPEA/DCM (1:1:8) was added to cap the remaining 2-chlorotrityl chloride bound to the resin. The mixture was agitated for 0.5 h. Then the solvent was drained and the resin was washed with DMF (4×3 mL). Preferably, resins were used for further peptide synthesis, where the loading with amino acids is <0.30 mmol/g. The following protocols were used:

Method A) Removal of the Fmoc group. A solution of 20% piperidine in DMF (3 mL) was added to the resin (1 g; loading<0.30 mmol/g) and the resulting suspension was shaken for 10 min. Then the solution was removed from the resin. Again, a solution of 20% piperidine in DMF (3 mL) was added to the resin and the resulting suspension was shaken for another 10 min. The solution was drained and the resin was washed with DMF (6×3 mL).

Method B) Amino acid coupling. Amino acid (4.0 eq) and TBTU (4.0 eq) were dissolved in dry DMF (3 mL). DIPEA (12 eq) was added dropwise to the DMF solution. After activating for 1 min, the resulting solution was added to the Fmoc-deprotected resin (1 g; loading<0.30 mmol/g). The mixture was shaken until the coupling reaction was completed. Then, the solution was drained and the resin was rinsed with DMF (4×3 mL).

All linear peptides with different amino acid sequences were synthesized using this protocol with an alternating sequence of Fmoc-deprotections (Method A) and amino acid couplings (Method B).

I₂-Mediated Cyclization

A) Solid Phase Cyclization

The thioether bridge (tryptathionine motif) was obtained from linear resin-bound peptide (1 eq.; (500 mg; loading<0.30 mmol/g)) synthesized according to the above method. Formation of the thioether was achieved by adding a freshly prepared solution of iodine in DMF (2 eq., 2 mg/ml) under protecting gas atmosphere (Ar or nitrogen). The mixture was shaken under nitrogen or argon atmosphere for 2.5 h to complete the formation of the thioether. If required also longer reaction times were applied (HPLC-MS control of a test cleavage). Then, the solution was drained and the resin was rinsed with DMF (4×3 mL).

B) Solution Phase Cyclization

To a freshly prepared solution of iodine in DMF (1 eq, 2 mg/ml) was added the linear peptide (1 eq) under Argon or nitrogen atmosphere. Then, the solution was stirred for 12 h to complete the formation of thioether. (HPLC-MS control!).

Cleavage from Solid Support

Condition A) The resin (500 mg; loading<0.30 mmol/g) was treated with 10 mL of a mixture of TFA/TIS/H₂O (95:2.5:2.5) for 1 h at room temperature with gentle agitation. The resin was filtered and rinsed with 1% TFA in DCM (2×5 mL). The rinses and filtrate were combined and evaporated to dryness.

Condition B) The resin (500 mg; loading<0.30 mmol/g) was treated with 10 mL of a mixture of TFE/HOAc/DCM (1:1:8) for 2 h at room temperature with gentle agitation. The resin was filtered and rinsed with DCM (2×5 mL). The rinses and filtrate were combined and evaporated to dryness.

Condition C) (test cleavage): in order to investigate completion of a cleavage reaction, 1-2 mg of resin were taken out of the reaction mixture, submitted to a plastic pipette equipped with a frit. Then the resin was washed each with 1-2 ml of DMF (2×), DCM (2×) and dried. TFA (0.25 ml) was added to the resin and incubated for 5 min. Then the TFA was removed at the vacuum and the residue was taken up for HPLC-MS analytics.

Macrolactamization

To a solution of monocyclic peptide (1 eq) in a solution of DIPEA (5 eq) in DMF, HATU (2 eq) was added at 0° C. The solution was stirred for 12 h, followed by preparative HPLC purification. The isolated product was lyophilized to give a white solid.

Deprotection of Protection Groups

Debenzylation: The final benzyl-protected amanitins or derivatives were dissolved in EtSH (10 mg/ml) and flushed with N₂ followed by addition of BF₃/Et₂O (10 eq) and were stirred for 30 min. Afterwards, the reaction mixture was evaporated under reduced pressure and the followed crude product was purified by preparative HPLC to give the final S-Deoxo amanitins.

Desilylation: Method A: To TBDMS-protected S-deoxo amanitins or derivatives in MeCN (10 mg/ml) was added BF₃/Et₂O (10 eq) and stirred for 10-30 min. The resulting crude product was then directly purified by preparative HPLC and the isolated product was lyophilized to give a white powder. Method B: To the protected TBDMS-S-deoxo amanitins in THE (10 mg/ml) was added a TBAF (10 eq) and stirred for 30 min-2 h. The resulting crude product was removed the solvent and subsequently precipitated in Et₂O. After centrifugation, the precipitate was directly purified by preparative HPLC and the isolated product was lyophilized to yielding a white powder.

Oxidation

Tryptathionin-containing peptide was dissolved in iPrOH/EtOH 2:1 (50 μL). To the resulting solution at room temperature was added mCPBA (1.0 eq). The progress of the reaction was monitored by HPLC-ESI-MS. Upon completion of the reaction (30 min), the crude reaction mixture was diluted in the HPLC buffer and directly purified on HPLC to afford a mixture (based on HPLC peaks integration) of (R)-sulfoxide and (S)-sulfoxide which could be separated by preparative HPLC.

Preparation of Tryptathionine Bridges by I₂-Mediated Cyclisation Using THP-Resin

Step 1: Fmoc-Hyp-OAII (19)

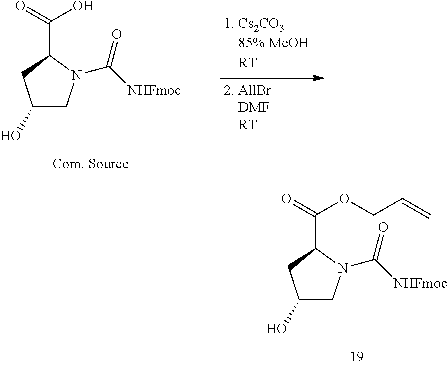

Fmoc-Hyp-OH (10.00 g, 28.3 mmol) was suspended in 100 ml 80% MeOH and Cs₂CO₃ (4.62 g, 14.1 mmol) was added. The suspension was stirred at room temperature for 15 minutes until complete dissolution. The reaction mixture was concentrated to dryness and suspended in 100 ml DMF. Allylbromide (2.6 ml, 3.6 g, 29.7 mmol) was added dropwise and the reaction was stirred overnight at room temperature. DMF was distilled off and the residue suspended in tert-butylmethyl ether. Precipitates were filtered and the clear solution was absorbed on Celite prior column chromatography. The compound was purified on 330 g silicagel with n-hexane/ethylacetate gradient.

Yield: 9.85 g, 89%

Step 2: THP-Resin Loading

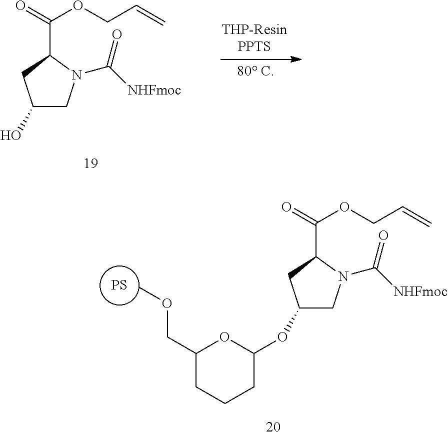

Amino acid 19 (9.85 g, 25.0 mmol), pyridinium 4-toluenesulfonate (2.62 g, 10.4 mmol) were added to a suspension of 1,3-dihydro-2H-pyran-2-yl-methoxymethyl resin (10.0 g, 0.77 mmol/g THP-resin) in 80 ml dichloroethane. The reaction was stirred at 80° C. overnight. After cooling, the resin was filtered and extensively washed with dichloroethane, dimethylformamide, acetonitrile, dichloromethane and tert-butylmethyl ether.

Loading was 0.60 mmol/g (determined by UV-spectroscopy of the fluorenemethyl group after deprotection)

Step 3: Linear Precursor Peptides by Solid Phase Synthesis

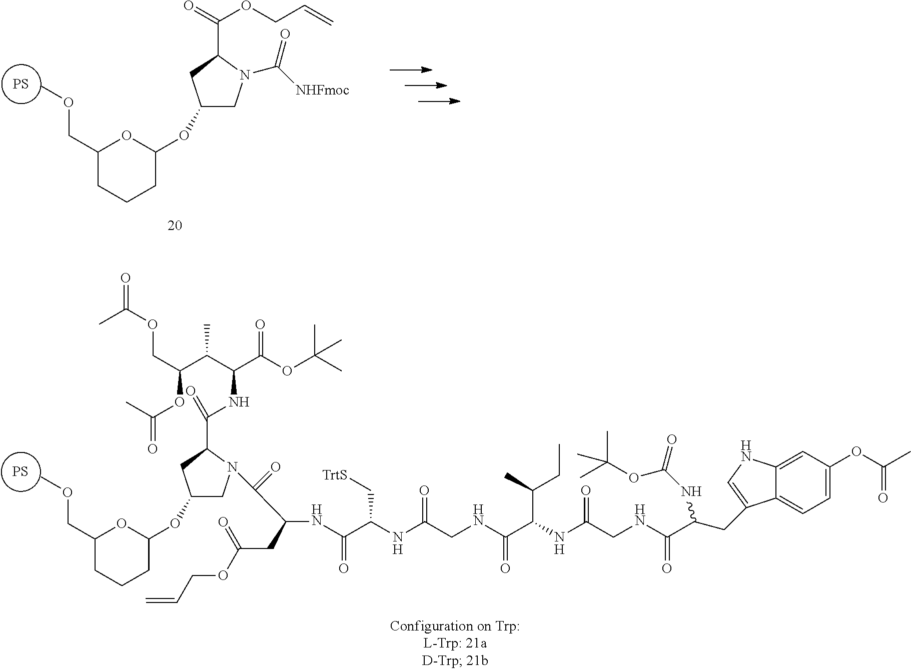

Resin Pre-Treatment:

Loaded resin 20 (3.20 g, 1.92 mmol) was treated with N,N-dimethylbarbituric acid (5.51 g, 35.3 mmol) and Pd(PPh₃)₄ (1.50 g, 1.30 mmol). The resin was shaken overnight at room temperature. Thereafter, the resin was extensively washed with dichloromethane, DMF, acetonitrile, dichloromethane and tert-butylmethyl ether and dried under reduced pressure.

Coupling Procedure:

All reactants and reagents were dissolved in dichloromethane/DMF (1:1, v/v) and the synthesis of the linear peptide performed using a CEM Liberty Blue Peptide Synthesizer in a 0.5 mmol scale. Stock solutions of amino acids/HOBt (0.3 N each), PyBOP (0.5 N) and DIEA (2 N) were used. Amino acids: HOBt:PyBOP:DIEA were used in a ratio of 1:1:1:2 using 2-5 equiv. of amino acid. The peptide coupling was conducted at 50° C. for 10 minutes by microwave irradiation (50 W, CEM microwave reactor) and was washed with DMF after coupling.

Fmoc-Deprotection:

Deprotection was performed by addition of 10.0 ml 20% Piperidine in N,N-dimethylformamide at 50° C. for 10 minutes. The resin was washed with DMF (no deprotection after coupling of the final amino acid as it was used as N³-Boc-protected derivative).

After completion, the resin was finally transferred into a syringe with bottom frit, washed with DCM and dried under reduced pressure.

Step 4: Iodine-Mediated Tryptathione Formation

General Procedure for Iodine-Mediated Cyclisation of Deoxy-Amanitin Precursors

Deoxy-L-6-Acetoxytryptophan-Amanitin Precursor 22a

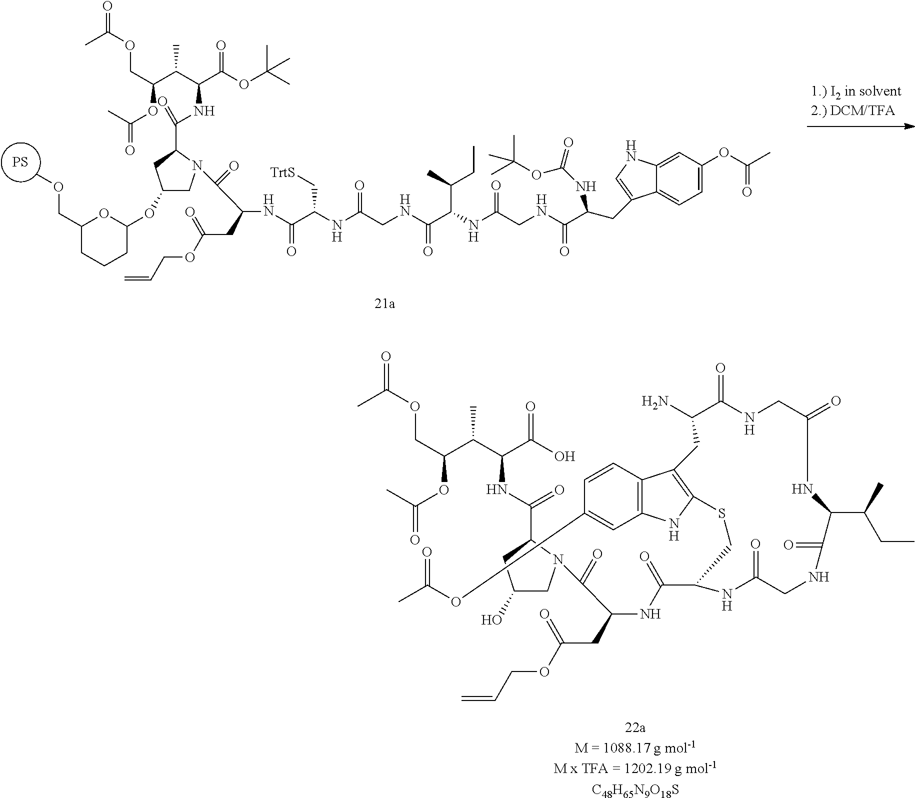

The THP resin-bound linear precursor peptide 21a (100 μmol, 1.0 equiv.) was swollen in solvent (either 15.6 mL TFE/water 9:1 or 96 mL DCM/TFA 95:1) and a solution of iodine (100 μmol, 1.0 equiv.) in DCM (4.0 mL) was added to the resulting suspension in portions over a period of 5 min in air The suspension was stirred for 2 h (solvent=DCM/TFA) or 19 h (solvent=TFE/water) at room temperature. Ethandithiol (85 μL, 1.0 mmol, 10 equiv., EDT) was added to quench the reaction and the suspension filtered. The resin was washed with dichloromethane (3×1 min) and the combined organic layers were concentrated in vacuo. The residue was treated with 5.0 mL DCM/TFA (1:1) for 3 h at room temperature. The brownish suspension was dropped into an ice cold solution of methyl-tert-butylether/hexane (1:1, 45 mL). The ether-peptide-suspension was incubated for 30 min at −20° C., the suspension spun down and the precipitate washed once with ice cold methyl-tert-butylether/hexane (1:1, 80 mL). The isolated precipitate was taken up in 5.0 mL of acetonitrile/water 3:1 (+0.05% TFA) and insoluble particles removed by centrifugation. The precipitate was treated as just described for additional two times and the combined filtrates were freeze-dried. The resulting crude peptide was purified by preparative RP-HPLC affording the monocyclic peptide 22a (solvent TFE/water: 34.7 mg, 28.9 μmol, 29% or solvent DCM/TFA: 31.6 mg, 26.3 μmol, 26% based on initial resin loading) as colourless TFA-salt. A side product (28.8-31.9 mg) was obtained and characterized as the deprotected disulfide. RP-HPLC: t_{R}=12.3 min, 89.5-99.9% purity (210 nm). MS (ESI, pos. mode): calc.: m/z=1088.4 [M+H]⁺, found: m/z=1089.2 [M+H]⁺

Deoxy-D-6-Acetoxytryptophan-Amanitin Precursor 22b

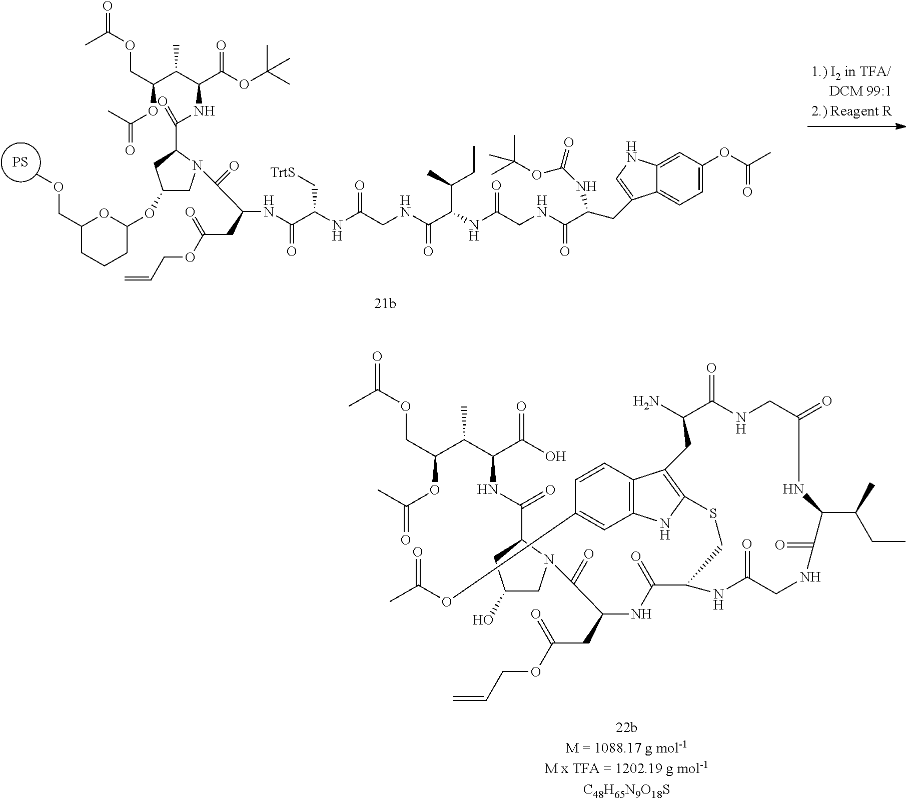

The THP resin-bound linear precursor peptide 21b (250 μmol, 1.0 equiv.) was swollen in dichloromethane (240 mL) and trifluoroacetic acid (TFA, 2.5 mL) was carefully added dropwise. To the resulting yellow suspension was added a solution of iodine (64.3 mg, 250 μmol, 1.0 equiv.) in dichloromethane (10 mL) in portions over a period of 5 min in air. The resulting peptide concentration was 1 mM. The suspension was stirred for 2 h at room temperature. Subsequently, ethanedithiol (50 μL, 594 μmol, 2.3 equiv.) and TIS (250 μL) were added to quench the reaction. The suspension was filtered and the resin washed with dichloromethane (3×1 min). The combined organic layers were concentrated in vacuo and the residue treated with 4.0 mL of reagent R (TFA:Thioanisol:EDT:Anisol 90:5:3:2) for 60 min at room temperature. The suspension was dropped into an ice cold solution of methyl-tert-butylether/hexane (1:1, 80 mL) and incubated for 30 min at −20° C. The suspension was spun down and the precipitate washed once with ice cold methyl-tert-butylether/hexane (1:1, 80 mL). The isolated precipitate was taken up in acetonitrile/water 3:1 (+0.05% TFA) and insoluble particles removed by centrifugation. The precipitate was treated as just described for additional two times and the combined filtrates were freeze-dried. The resulting crude peptide (262.4 mg) was purified by preparative RP-HPLC affording the monocyclic peptide 22b (95.1 mg, 79.1 μmol, 32% based on initial resin loading) as colourless TFA-salt. A side product (28.0 mg, 25.6 μmol, 10%) was obtained and characterized as the deprotected linear peptide resulting form the cleaved disulfide side product. RP-HPLC: t_{R}=12.2 min. MS (ESI, pos. mode): calc.: m/z=1088.4 [M+H]⁺, found: m/z=1089.0 [M+H]⁺

Analytical RP-HPLC

Column: Luna 10 μm C18(2), 100 A, 250×4.6 mm

Gradient: Solvent A=water (+0.05% TFA); solvent B=acetonitrile

0 to 25 min: from 5% to 81% B; 25 to 25.2 min: from 81% to 100% B; 25.2 to 27.5 min: 100% B; 27.5 to 28 min: from 100% to 5% B; 28 to 30 min: 5% B

Prep. RP-HPLC

Column: Luna 10 μm C18(2), 100 A, 250×21.2 mm

Gradient: Solvent A=water (+0.05% TFA); solvent B=acetonitrile

0 to 20 min: from 5% to 46% B; 20 to 20.2 min: from 46% to 100% B; 20.2 to 22.5 min: 100% B; 22.5 to 25 min: 5% B.

Assessment of Cytotoxicity

Cytotoxicity of compounds of the invention was assessed using an RNA Polymerase II inhibition assay and by assessing cytotoxicity in a cell viability assay on HEK cells and HEK-OATP1 B3 cells, the results of which are depicted in Table 6.

RNA Polymerase II Assay

The in vitro RNA Pol II assay was done using a HeLaScribe® Nuclear extract in vitro transcription system (Promega, #E3092) was used to compare the inhibitory effect of amanitin analogues on RNA Pol at seven concentrations from 6.4×10⁻¹¹ M to 1×10⁻⁶ M (serial 1:5 dilutions). As positive control, template run-off transcripts from a CMV immediate early promoter were used (Promega, #E3092). Reverse transcription was followed by real time-PCR for the quantification of the mRNA product. RNA detection was performed using a QuantiFast Probe RT-PCR Plus Kit (Qiagen). The PCR product was monitored by determination of fluorescence on a Real Time PCR CFX Connect Real Time System (Bio-Rad Laboratories Inc.). α-amanitin, analogues and untreated control were each analyzed in triplicates. Negative control, positive control and no target control were analyzed in duplicates. The A_{CT} method was used to calculate the inhibitory effect of the test item on transcription normalized to the untreated control.

Cell Viability Assay (HEK and HEK-OATP1B3 Cells)

The in vitro toxicity of the inventive compounds was determined in Human embryonic kidney HEK293 cells and in HEK293-OATP1B3 cells that overexpress the organic anion-transporting polypeptide 11B3 (OATP1B3) to assess whether OATP1 B3 mediates active transport of amanitin analogues into the cell. Wildtype HEK293 (ATCC, Manassas, Va.) and HEK293-OATP1 B3 cells (see: Pahl et al., Cytotoxic Payloads for Antibody-Drug Conjugates In: Amatoxins as RNA Polymerase II Inhibiting Antibody-Drug Conjugate (ADC) Payloads CHAPTER 19, The Royal Society of Chemistry 2019, ISBN: 978-1-78801-077-1, or e.g. Bowman et al. Drug Metab Dispos 48:18-24, January 2020) were incubated with compound 7f (α-amanitin) or the compounds as disclosed in Table 6. Cytotoxicity was determined by a BrdU assay. HEK293 and HEK-OATP1B3 cells were plated at 2.5×10³ cells/well in a 1:1 mixture of Ham's F12 with DMEM containing 10% charcoal-stripped FCS onto poly-D-lysine-coated 96-well plates and grown for 24 hours. Subsequently, cells were incubated with amanitin derivatives at 8 different concentrations (1×10⁻⁶ M to 1.28×10⁻¹¹ M, serial 1:5 dilutions). Following 96 h of drug exposure, cell viability was determined by a BrdU incorporation assay (Cell Proliferation ELISA, BrdU, Roche) and chemiluminescence was measured using a FLUOstar Optima plate reader (BMG LABtech). Data analysis was performed with GraphPad Prism 9.0 (GraphPad Software, Inc., La Jolla, Calif.) software to plot curve fits.

TABLE 6
Cytotoxicity

| | | | | Cytotoxicity on |
|---|---|---|---|---|
| | | RNAP II | Cytotoxicity on | HEK-OATP1B3 |
| | Modification | inhibition | HEK-293 cells | cells |
| compound | site | IC₅₀ (M) | EC₅₀ (M) | EC₅₀ (M) |
| 7f (α-amanitin) | | 2.7 × 10⁻⁹ | 2.1 × 10⁻⁷ | 5.2 × 10⁻⁹ |
| 7d (Ama-11) | — | 6.8 × 10⁻⁹ | 8.1 × 10⁻⁸ | 2.3 × 10⁻¹⁰ |
| 7i (Ama-12) | D-Pro⁷ | 1.2 × 10⁻⁸ (20%)* | >1 × 10⁻⁶ | 2.1 × 10⁻⁷ |
| 7l (Ama-13) | Sar⁷ | 1.5 × 10⁻⁸ (10%)* | >1 × 10⁻⁶ | 2.9 × 10⁻⁸ |
| 7m (Ama-15) | Aib⁷ | 7.1 × 10⁻⁸ (15%)* | >1 × 10⁻⁶ | 1.2 × 10⁻⁷ |
| 7n (Ama-23) | Aze² | 1.6 × 10⁻⁷ (40%)* | >1 × 10⁻⁶ | >1 × 10⁻⁷ |
| 7o (Ama-27) | D-Ala⁷ | 1.4 × 10⁻⁹ | >1 × 10⁻⁶ | 2.8 × 10⁻⁸ |
| 7p (Ama-32) | Tle⁶ | 2.5 × 10⁻⁸ | >1 × 10⁻⁶ | 1.6 × 10⁻⁸ |
| 7q (Ama-38) | 4-(F₂)Pro² | 1.1 × 10⁻⁷ (10%)* | >1 × 10⁻⁶ | 4.9 × 10⁻⁸ |
| 7r (Ama-44) | Acc⁷ | 2.6 × 10⁻⁸ | >1 × 10⁻⁶ | 1.0 × 10⁻⁸ |

*indicates residual RNAP II activity at highest concentration tested

### REFERENCES

1. T. Wieland, Peptides of Poisonous Amanita Mushrooms (Eds.: A. Rich), Wiley-VCH, Weinheim, 1986.
2. A. Pahl et al. Drug Discov Today: Technol, 2018, 30, 85-89.
3. M. O. Anderson, A. A. Shelat, R. K. Guy, J Org Chem 2005, 70, 4578-4584.
4. W. E., Savige, A. Fontana, J. Chem. Soc., Chem. Commun. 1976, 600-601.
5. G. Zanotti, L. Falcigno, M. Saviano, G. D'Auria, B. M. Bruno, T. Campanile, L. Paolillo, Chem-Eur J 2001, 7, 1479-1485.
6. K. Matinkhoo, A. Pryyma, M. Todorovic, B. O. Patrick, D. M. Perrin, J Am Chem Soc 2018, 140, 6513-651.
7. P. Sieber, B. Kamber, B. Riniker, W. Rittel, Helv Chim Acta 1980, 63, 2358-2363.
8. L. A. Schuresko, R. S. Lokey, Angew Chem Int Edit 2007, 46, 3547-3549.
9. G. Yao, J. O. Joswig, B. G. Keller, R. D. Süssmuth, Chem-Eur J 2019, 25, 8030-8034.
10. Edelman, G. M. et al., Proc. Natl. Acad. USA, 63, 78-85 (1969)
11. Andreas Pahl, Christian Lutz, Torsten Hechler: Amatoxins as RNA Polymerase II Inhibiting Antibody-Drug Conjugate (ADC) Payloads, CHAPTER 19, The Royal Society of Chemistry 2019, ISBN: 978-1-78801-077-1 doi: 10.1039/9781788012898-00398
12. Bowman et al. Drug Metab Dispos 48:18-24, January 2020
13. Binz et al. Nat Biotechnol 2005 October; 23(10):1257-68
14. Jain et al. Pharm Res (2015) 32:3526-3540
15. Dubowchik et al., Bioconjug Chem. 13 (2002) 855-69
16. Peeters et al. J Immunol Methods. 1989 Jun. 2; 120(1):133-43
17. Carlsson et al. Biochem J. 1978; 173:723-37.

1. A method for preparation of a compound of formula (I) 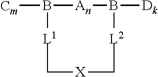
wherein a compound of formula (II) 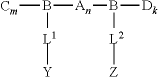
is reacted with iodine (I₂), N-bromosuccinimide, or N-iodosuccinimide, Iodine monochloride, Iodine monobromide and Bis(pyridine)iodonium tetrafluoroborate, particularly with iodine, in a reaction step (a) yielding a compound of formula (I); wherein
Z is selected from SH, S-trityl (STrt), S-acetamidomethyl (SAcm), S-diphenylmethyl (SDpm), S-monomethoxytrityl (SMmt), and S-tert-butyl (S^{t}Bu); particularly selected from SH, S-trityl (STrt), S-acetamidomethyl (SAcm), S-diphenylmethyl (SDpm), S-monomethoxytrityl (SMmt), and S-tert-butyl (S^{t}Bu), most particularly Z is STrt;
Y is an unsubstituted or hydroxyl-, halogen-, halogenated carbon-, alkynyl-, olefin-, cyano-, protected carboxylate and/or carboxyamide-substituted indole, particularly is an unsubstituted or hydroxyl-, halogen-, halogenated carbon-, alkynyl-, olefin-, cyano-, protected carboxylate and/or carboxyamide-substituted indole;
L¹ is an unsubstituted or Me-substituted C₁-C₂ alkyl linker, particularly L¹ is CH₂;
L² is an unsubstituted or Me-substituted C₁-C₂ alkyl linker, particularly L¹ is CH₂ or —C(CH₃)₂—;
B is an α-amino acid, a j-amino acid backbone, a α-methyl amino acid, particularly B is an α-amino acid backbone (NH—CHR—C═O with R being L¹ or L²);
every A is independently selected from a proteinogenic and a non-proteinogenic α-amino acid in L- or D-conformation or a j-amino acid, particularly every A is independently selected from a proteinogenic and a non-proteinogenic α-amino acid in L- or D-conformation;
n is an integer selected from 2, 3, and 4;
with the proviso that the ring 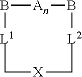
consists of 14 to 26 bonds, particularly of 15 to 21 bonds, more particularly of 18 bonds;
every C and D are independently selected from a proteinogenic and a non-proteinogenic α-amino acid in L- or D-conformation, wherein either C or D of formula (II) is connected to a resin or has a protected N-terminus;
m and k are independently selected from an integer between 0 and 4, particularly wherein the sum of m and k is an integer between 0 and 4;
X is either (-(-indole-S—) or (—S-indole-)-) with the indole being an unsubstituted or hydroxyl-, halogen-, halogenated carbon-, alkynyl-, olefin-, cyano-, protected carboxylate and/or carboxyamide-substituted indole,
wherein optionally the sulphur atom of X can be subsequently oxidized.

2. The method according to claim 1, wherein either C or D of formula (II) is connected to a resin and is reacted with iodine (I₂).

3. The method according to claim 1, wherein either C or D of formula (II) has a protected N-terminus and is reacted with iodine (I₂).

4. The method according to claim 1, wherein the reaction step (a) is performed in a polar solvent.

5. The method according to claim 1, wherein iodine is used at a concentration of 1-4 mg/ml.

6. The method according to claim 1, wherein n is 3.

7. The method according to claim 1, wherein A is independently selected from a proteinogenic or non-proteinogenic α-amino acid.

8. The method according to claim 1, wherein C and D are independently selected from a proteinogenic or non-proteinogenic α-amino acid.

9. The method according to claim 1, wherein the indole of Y is unsubstituted or substituted with one, two, three or four groups selected from hydroxyl, halogen, CN and a fluorinated carbon.

10. The method according to claim 1, wherein said resin is an acid labile resin.

11. The method according to claim 1, wherein the sulphur atom of Z is oxidized.

12. A method for preparation of a compound of formula (VI) 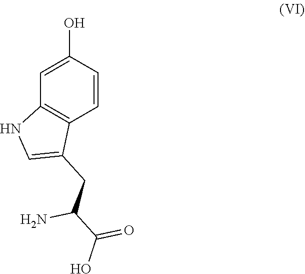
wherein a compound of formula (VII) 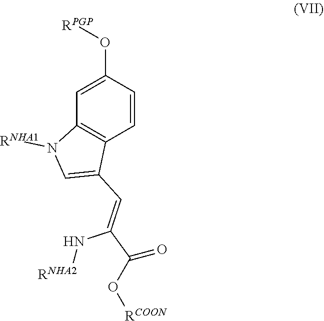
wherein
R^{NHA1} is an amino protecting group,
R^{NHA2} is an amino protecting group,
R^{PGP} is a protecting group for phenolic OH groups, and
R^{COON} is a carboxyl-protecting group,
is reacted with H₂, an organometallic rhodium or ruthenium complex and organophosphorus ligand, in a reaction step (a) and
the compound is reacted with a deprotection agent removing R^{NHA1}, R^{NHA2}, R^{PGP}, and R^{COON},
to yield the compound characterized by (VI).

13. The method according to claim 12, wherein a compound of formula (VIII) 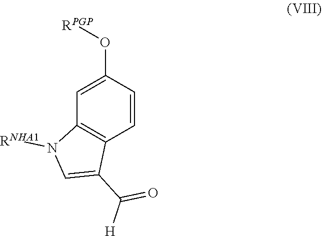
wherein
R^{NHA1} and R^{PGP} have the same meanings as described in claim 12, is reacted with a protected 2-phosphonoglycine-methyl dimethyl ester under basic conditions in a reaction step (b)
to yield the compound characterized by (VII).

14. A compound of formula (IIIa), (IIIb), 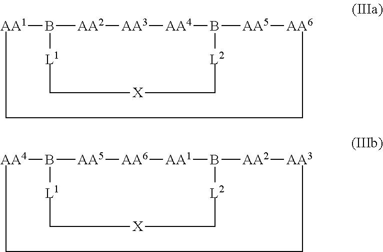
or a compound of formula (IVa), (IVb), (IVc), (IVd), (IVe), (IVf), (IVg), or (IVh) 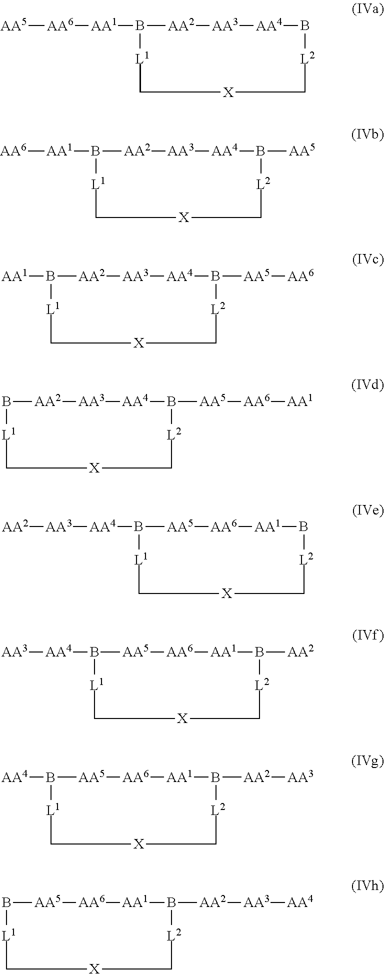
wherein
X is either of formula Q-R in formula [(IVa), (IVb), (IVc) and (IVd)], or R-Q in formula [(IVe), (IVf), (IVg) and (IVh)], wherein
Q is unsubstituted or CF₃-, alcohol-, alkyl-, O-alkyl-, hydroxyl-, and/or halogen-substituted imidazole or indole;
R is S, SO, or SO₂;
L¹ is an unsubstituted or Me-substituted C₁-C₂ alkyl linker, particularly L¹ is an unsubstituted C₁ alkyl linker;
L² is an unsubstituted or Me-substituted C₁-C₂ alkyl linker;
B is an α-amino acid backbone;
AA¹ is selected from DHIle (dihydroxy-isoleucin), Ile, DHLeu (dihydroxy-leucin), HVal (hydroxy-valin), HIle (5-hydroxy-isoleucin or 4-hydroxyisoleucine), Gly, D-Pro, L-Pro, D-Ala, Aib (2-aminoisobutyric acid), and Ala;
AA² is selected from Gly, D-Pro, D-Ala, Aib (2-aminoisobutyric acid), Asn, Asp, Ser, Cys, Arg, Lys, Gln, Glu, and L-Propargyl-Gly;
AA³ is selected from Ile, Leu, Val, PhotoLeu, and L-Propargyl-Gly, L-propargy-Gly, Pro, Gly, Aib, trans-4-Hyp, Hyp, 4-F-Pro, 4-NH₂-Pro, Photo-Pro, L-Pipecolinic acid, L-azetidine-2-carboxylic acid, (S)-Indoline-2-carboxylic acid, L-4-Thiazolidinecarboxylic acid, Tle;
AA⁴ is selected from Gly, D-Pro, D-Ala, Aib (2-aminoisobutyric acid), Asn, Asp, Ser, Cys, Arg, Lys, Gln, Glu, and L-Propargyl-Gly, Sar, Acc;
AA⁵ is selected from Asn, Asp, Ser, Cys, Arg, Lys, Gln, Glu, and L-Propargyl-Gly, D-Pro, D-Ala, Aib (2-aminoisobutyric acid), and Ala; and
AA⁶ is selected from Pro, Gly, Aib, trans-4-Hyp, Hyp, 4-F-Pro, 4-F₂-Pro 4-NH₂-Pro, Photo-Pro, L-Pipecolinic acid, L-azetidine-2-carboxylic acid, (S)-Indoline-2-carboxylic acid, L-4-Thiazolidinecarboxylic acid, Ile, Leu, Val, PhotoLeu, and L-Propargyl-Gly; and
wherein in formula (IVa), (IVb), (IVc), (IVd), (IVe), (IVf), (IVg), or (IVh) the C-terminal amino acid is optionally connected to a resin.

15. The compound according to claim 14, wherein said compound is not composed of the following combinations:
Q is unsubstituted or alkyl-, O-alkyl-, hydroxyl-, and/or halogen-substituted indole;
R is S, SO, or SO₂;
L¹ is CH₂;
L² is CH₂;
AA¹ is selected from DHIle (dihydroxy-isoleucin), Ile, and HIle (hydroxy-isoleucin);
AA² is Gly;
AA³ is Ile;
AA⁴ is Gly;
AA⁵ is Asn or Asp; and
AA⁶ is Pro or Hyp (hydroxyproline);
or
Q is unsubstituted or alkyl-, O-alkyl-, hydroxyl-, and/or halogen-substituted indole;
R is S, SO, or SO₂;
L¹ is CH₂;
L² is CH₂;
AA¹ is DHIle or Ile;
one of AA² and AA⁴ is Gly and the other one is Ala, or both AA² and AA⁴ are Ala;
AA³ is Ile;
AA⁵ is Asn; and
AA⁶ is Hyp (hydroxyproline);
or
Q is unsubstituted or alkyl-, O-alkyl-, hydroxyl-, and/or halogen-substituted indole;
R is S, SO, or SO₂;
L¹ is CH₂;
L² is CH₂;
AA¹ is selected from DHLeu (dihydroxy-leucin), and HVal (hydroxy-valin);
AA² is Gly;
AA³ is Ile;
AA⁴ is Gly;
AA⁵ is Asn; and
AA⁶ is Hyp (hydroxyproline).

16. The compound according to claim 14, wherein AA² is Gly and/or AA⁴ is Gly.

17. The compound according to claim 14, wherein
AA² is Gly and AA⁴ is D-Pro; or
AA² is D-Pro or L-Pro and AA⁴ is Gly.

18. A method for the manufacture of an antibody-drug conjugate comprising use of a compound according to claim 14.

19. The method according to claim 18, wherein the compound is selected from the group of compounds comprising 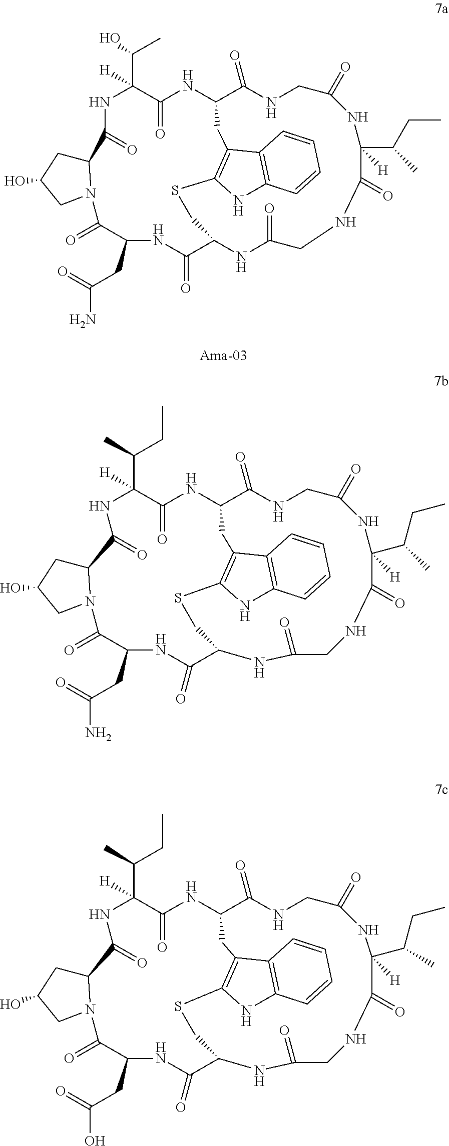 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)O)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(=O)O)C(=O)N1C[C@H](O)C[C@H]1C(=O)N[C@@H]([C@@H](C)CC)C(=O)N2] 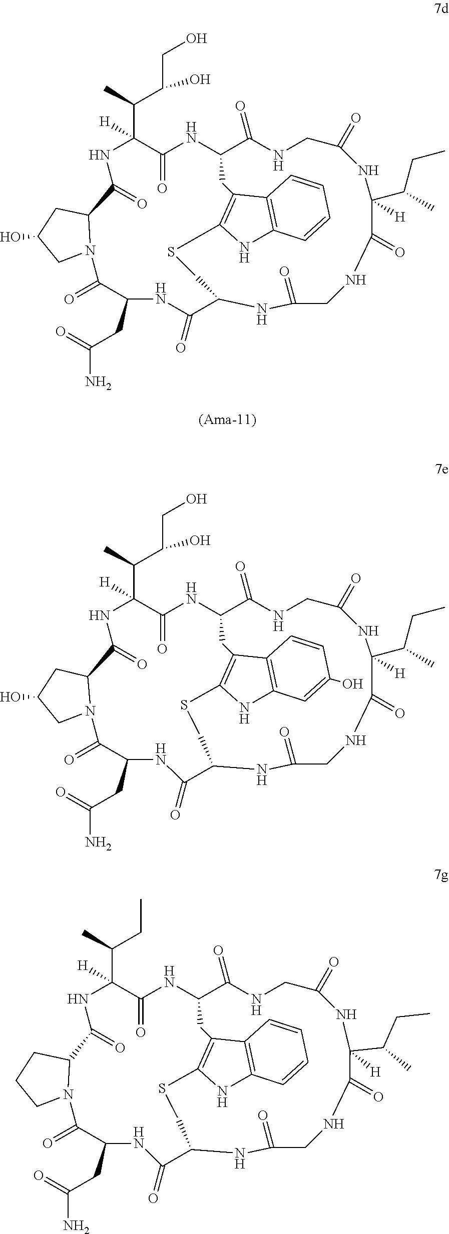 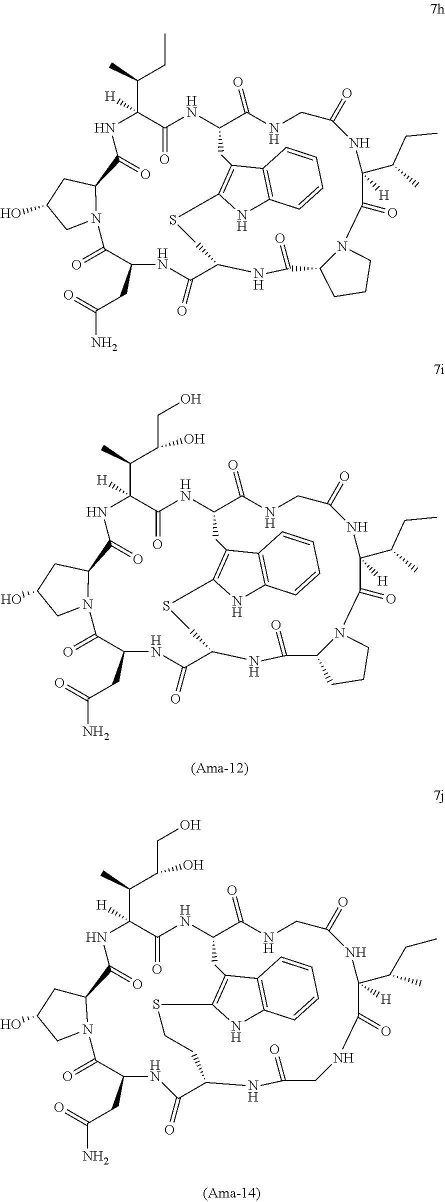 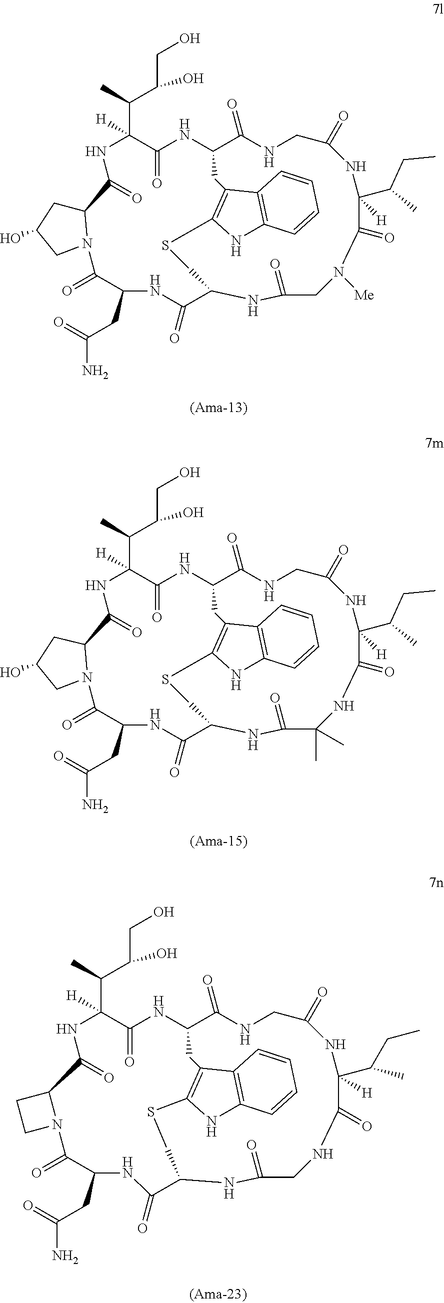 [SMILES: CC[C@H](C)[C@@H]1NC(=O)CNC(=O)[C@@H]2Cc3c([nH]c4ccccc34)SC[C@H](NC(=O)CN(C)C1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4ccccc34)SC[C@H](NC(=O)C(C)(C)NC1=O)C(=O)N[C@@H](CC(N)=O)C(=O)N1C[C@H](O)C[C@H]1C(=O)N[C@@H]([C@@H](C)[C@@H](O)CO)C(=O)N2 ; CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1CC[C@H]1C(=O)N[C@@H]([C@@H](C)[C@@H](O)CO)C(=O)N2] 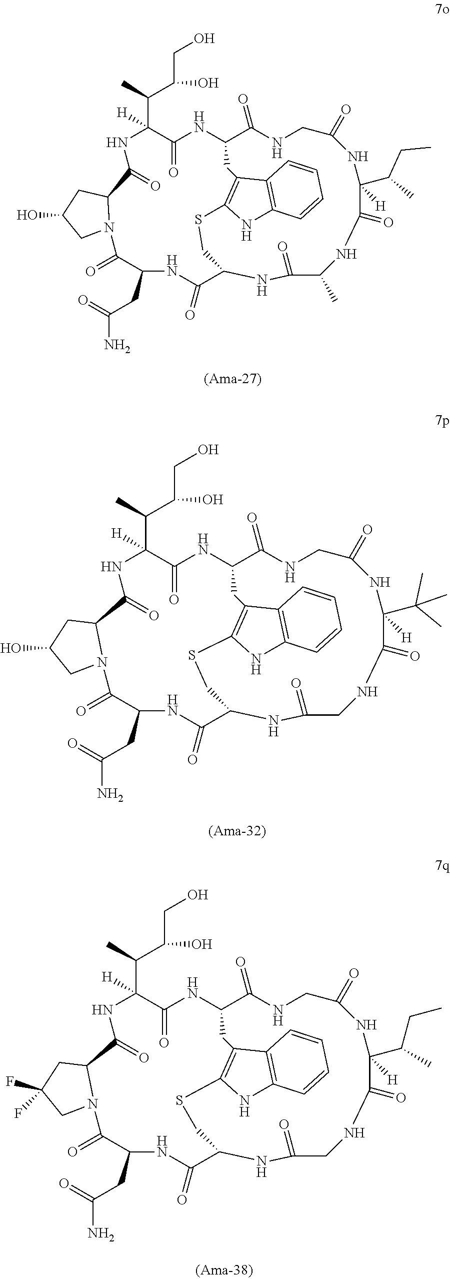 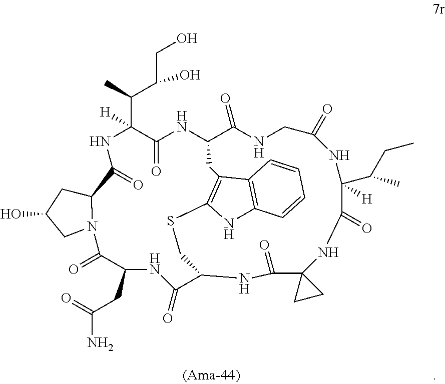 [SMILES: CC[C@H](C)[C@@H]1NC(=O)CNC(=O)[C@@H]2Cc3c([nH]c4ccccc34)SC[C@H](NC(=O)C3(CC3)NC1=O)C(=O)N[C@@H](CC(N)=O)C(=O)N1C[C@H](O)C[C@H]1C(=O)N[C@@H]([C@@H](C)[C@@H](O)CO)C(=O)N2]
